SEQUENCE LISTING (71 sequences)

SEQ ID NO: 1 - DNA, 24 bp, Artificial Sequence
  Synthetic Construct
tcgtcgttttgtcgttttgtcgtt

SEQ ID NO: 2 - DNA, 20 bp, Artificial Sequence
  Synthetic Construct
tccatgacgttcctgacgtt

SEQ ID NO: 3 - protein, 273 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 4 - skipped

SEQ ID NO: 5 - DNA, 20 bp, Artificial Sequence
  Synthetic Construct
ggggtcaacgttgagggggg

SEQ ID NO: 6 - DNA, 20 bp, Artificial Sequence
  Synthetic Construct
gggggacgatcgtcgggggg

SEQ ID NO: 7 - DNA, 21 bp, Artificial Sequence
  Synthetic Construct
ggggacgacgtcgtggggggg

SEQ ID NO: 8 - DNA, 22 bp, Artificial Sequence
  Synthetic Construct
tcgtcgttgtcgttttgtcgtt

SEQ ID NO: 9 - DNA, 23 bp, Artificial Sequence
  Synthetic Construct
tcgacgttcgtcgttcgtcgttc

SEQ ID NO: 10 - DNA, 26 bp, Artificial Sequence
  Synthetic Construct
tcgcgacgttcgcccgacgttcggta

SEQ ID NO: 11 - DNA, 22 bp, Artificial Sequence
  Synthetic Construct
tcgtcgttttcggcgcgcgccg

SEQ ID NO: 12 - DNA, 25 bp, Artificial Sequence
  Synthetic Construct
tcgtcgtcgttcgaacgacgttgat

SEQ ID NO: 13 - DNA, 29 bp, Artificial Sequence
  Synthetic Construct
tcgcgaacgttcgccgcgttcgaacgcgg

SEQ ID NO: 14 - protein, 1255 aa, Severe acute respiratory syndrome coronavirus 2
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 15 - DNA, 22 bp, Artificial Sequence
  Synthetic Construct
tgactgtgaacgttcgagatga

SEQ ID NO: 16 - protein, 1255 aa, Severe acute respiratory syndrome coronavirus 2
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHAGQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 17 - protein, 180 aa, Severe acute respiratory syndrome coronavirus 2
CPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFE

SEQ ID NO: 18 - protein, 4 aa, Artificial Sequence
  Synthetic Construct
GGGA

SEQ ID NO: 19 - protein, 4 aa, Artificial Sequence
  Synthetic Construct
GGGG

SEQ ID NO: 20 - protein, 5 aa, Artificial Sequence
  Synthetic Construct
GGGAG

SEQ ID NO: 21 - protein, 6 aa, Artificial Sequence
  Synthetic Construct
GGGAGG

SEQ ID NO: 22 - protein, 7 aa, Artificial Sequence
  Synthetic Construct
GGGAGGG

SEQ ID NO: 23 - protein, 4 aa, Artificial Sequence
  Synthetic Construct
GGAG

SEQ ID NO: 24 - protein, 4 aa, Artificial Sequence
  Synthetic Construct
GGSG

SEQ ID NO: 25 - protein, 4 aa, Artificial Sequence
  Synthetic Construct
AGGG

SEQ ID NO: 26 - protein, 4 aa, Artificial Sequence
  Synthetic Construct
SGGG

SEQ ID NO: 27 - protein, 6 aa, Artificial Sequence
  Synthetic Construct
GGAGGA

SEQ ID NO: 28 - protein, 6 aa, Artificial Sequence
  Synthetic Construct
GGSGGS

SEQ ID NO: 29 - protein, 9 aa, Artificial Sequence
  Synthetic Construct
GGAGGAGGA

SEQ ID NO: 30 - protein, 9 aa, Artificial Sequence
  Synthetic Construct
GGSGGSGGS

SEQ ID NO: 31 - protein, 12 aa, Artificial Sequence
  Synthetic Construct
GGAGGAGGAGGA

SEQ ID NO: 32 - protein, 12 aa, Artificial Sequence
  Synthetic Construct
GGSGGSGGSGGS

SEQ ID NO: 33 - protein, 8 aa, Artificial Sequence
  Synthetic Construct
GGAGGGAG

SEQ ID NO: 34 - protein, 8 aa, Artificial Sequence
  Synthetic Construct
GGSGGGSG

SEQ ID NO: 35 - protein, 12 aa, Artificial Sequence
  Synthetic Construct
GGAGGGAGGGAG

SEQ ID NO: 36 - protein, 12 aa, Artificial Sequence
  Synthetic Construct
GGSGGGSGGGSG

SEQ ID NO: 37 - protein, 15 aa, Artificial Sequence
  Synthetic Construct
GGGGAGGGGAGGGGA

SEQ ID NO: 38 - protein, 15 aa, Artificial Sequence
  Synthetic Construct
GGGGSGGGGSGGGGS

SEQ ID NO: 39 - protein, 10 aa, Artificial Sequence
  Synthetic Construct
YSVLYNSASF

SEQ ID NO: 40 - protein, 9 aa, Artificial Sequence
  Synthetic Construct
YQPYRVVVL

SEQ ID NO: 41 - protein, 9 aa, Artificial Sequence
  Synthetic Construct
VRFPNITNL

SEQ ID NO: 42 - protein, 9 aa, Artificial Sequence
  Synthetic Construct
FNATRFASV

SEQ ID NO: 43 - protein, 9 aa, Artificial Sequence
  Synthetic Construct
KIADYNYKL

SEQ ID NO: 44 - protein, 9 aa, Artificial Sequence
  Synthetic Construct
VSPTKLNDL

SEQ ID NO: 45 - protein, 10 aa, Artificial Sequence
  Synthetic Construct
VCGPKKSTNL

SEQ ID NO: 46 - protein, 10 aa, Artificial Sequence
  Synthetic Construct
SYGFQPTNGV

SEQ ID NO: 47 - protein, 9 aa, Artificial Sequence
  Synthetic Construct
VYAWNRKRI

SEQ ID NO: 48 - protein, 9 aa, Artificial Sequence
  Synthetic Construct
SFVIRGDEV

SEQ ID NO: 49 - protein, 8 aa, Artificial Sequence
  Synthetic Construct
VNFNFNGL

SEQ ID NO: 50 - protein, 10 aa, Artificial Sequence
  Synthetic Construct
KCYGVSPTKL

SEQ ID NO: 51 - protein, 9 aa, Artificial Sequence
  Synthetic Construct
CYGVSPTKL

SEQ ID NO: 52 - protein, 9 aa, Artificial Sequence
  Synthetic Construct
CYGVSATKL

SEQ ID NO: 53 - protein, 8 aa, Artificial Sequence
  Synthetic Construct
YGVSPTKL

SEQ ID NO: 54 - protein, 10 aa, Artificial Sequence
  Synthetic Construct
SKVGGNYNYL

SEQ ID NO: 55 - protein, 9 aa, Artificial Sequence
  Synthetic Construct
VIAWNSNNL

SEQ ID NO: 56 - protein, 9 aa, Artificial Sequence
  Synthetic Construct
ESIVRFPNI

SEQ ID NO: 57 - protein, 9 aa, Artificial Sequence
  Synthetic Construct
VVVLSFELL

SEQ ID NO: 58 - protein, 9 aa, Artificial Sequence
  Synthetic Construct
GNYNYLYRL

SEQ ID NO: 59 - protein, 9 aa, Artificial Sequence
  Synthetic Construct
VGYQPYRVV

SEQ ID NO: 60 - protein, 9 aa, Artificial Sequence
  Synthetic Construct
YNSASFSTF

SEQ ID NO: 61 - protein, 9 aa, Artificial Sequence
  Synthetic Construct
IAPGQTGKI

SEQ ID NO: 62 - protein, 8 aa, Artificial Sequence
  Synthetic Construct
GGGSGGGS

SEQ ID NO: 63 - protein, 432 aa, Artificial Sequence
  Synthetic Construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAENLYFQGHHHHHH

SEQ ID NO: 64 - protein, 7 aa, Artificial Sequence
  Synthetic Construct
ENLYFQG

SEQ ID NO: 65 - protein, 6 aa, Artificial Sequence
  Synthetic Construct
HHHHHH

SEQ ID NO: 66 - protein, 1216 aa, Artificial Sequence
  Synthetic Construct
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGGGSHHHHHHHHHH

SEQ ID NO: 67 - protein, 10 aa, Artificial Sequence
  Synthetic Construct
HHHHHHHHHH

SEQ ID NO: 68 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 69 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 70 - protein, 11 aa, Artificial Sequence
  Synthetic Construct
AHHHHHHHHHH

SEQ ID NO: 71 - DNA, 20 bp, Artificial Sequence
  Synthetic Construct
tccatgacgttcctgatgct

(19) United States
(12) Patent Application Publication
(10) Pub. No.: US 2023/0263882 A1
(43) Pub. Date: Aug. 24, 2023

(54) COMPOSITIONS AND METHODS FOR INDUCING AN IMMUNE RESPONSE AGAINST CORONAVIRUS

(71) Applicant: Elicio Therapeutics, Inc., Boston, MA (US)

(72) Inventors: Martin P. STEINBUCK, Boston, MA (US); Lochana M. SEENAPPA, Boston, MA (US); Peter C. DEMUTH, Boston, MA (US); Christopher M. HAQQ, Boston, MA (US); Lisa MCNEIL, Boston, MA (US)

(21) Appl. No.: 18/012,343

(22) PCT Filed: Jun. 25, 2021
(86) PCT No.: PCT/US2021/039134

§ 371 Date: Dec. 22, 2022

Related U.S. Application Data
(60) Provisional application No. 63/145,200, filed on Feb. 3, 2021.
(60) Provisional application No. 63/124,200, filed on Dec. 11, 2020.
(60) Provisional application No. 63/064,836, filed on Aug. 12, 2020.
(60) Provisional application No. 63/044,773, filed on Jun. 26, 2020.

(51) Int. Cl. A61K 39/215; A61P 31/14

(52) U.S. Cl. CPC A61K 39/215; A61P 31/14; A61K 2039/55561

(57) ABSTRACT

Disclosed herein are CpG-amphiphiles and corona virus antigens (e.g., a coronavirus spike protein, a peptide thereof, or a nucleic acid sequence encoding the same) for use in inducing an immune response in a subject, and methods of administering CpG-amphiphiles and coronavirus antigens (e.g., a coronavirus spike protein, a peptide thereof, a coronavirus nucleocapsid protein, a peptide thereof, or a nucleic acid sequence encoding the same) to induce an immune response in a subject.

### PRIORITY CLAIM

The present application claims benefit of the filing dates of U.S. Provisional Application No. 63/044,773, filed Jun. 26, 2020, U.S. Provisional Application No. 63/064,836, filed Aug. 12, 2020, U.S. Provisional Application No. 63/124,200, filed Dec. 11, 2020, and U.S. Provisional Application No. 63/145,200, filed Feb. 3, 2021, each of which is hereby incorporated herein by reference in its entirety.

### BACKGROUND OF THE INVENTION

Coronaviruses are a large family of viruses capable of infecting mammals and birds. The coronavirus family includes four genera: alpha-, beta-, gamma-, and deltacoronavirus. Coronavirus infections in humans usually cause mild to moderate upper-respiratory tract illnesses, like the common cold. Recently, coronavirus outbreaks, which have emerged from zoonotic spillover, are causing severe disease and global transmission concerns.

Up until 2019, six human coronaviruses were known, including the alphacoronaviruses (e.g., human coronavirus 229E (HCoV-229E) and human coronavirus NL63 (HCoV-NL63)) and the betacoronaviruses (e.g., human coronavirus OC43 (HCoV-OC43), human coronavirus-HKU1 (HCoV-HKU1), severe acute respiratory syndrome (SARS) associated coronavirus (SARS-CoV), and Middle East Respiratory Syndrome (MERS-CoV)). The 2019 novel betacoronavirus (SARS-CoV-2), which is the cause of the highly infectious disease known as COVID-19, emerged recently in China and has quickly spread worldwide, resulting in >7,690,708 confirmed cases and >427,630 deaths as of Jun. 14, 2020.

Based on hospitalized patient data, the majority of COVID-19 cases (about 80%) present with asymptomatic or mild symptoms, while the remainder are severe or critical (Huang et al., Lancet 395:497 (2020); Chan et al., Lancet 395:514 (2020)). Although the vast majority of patients experience only a mild form of the illness, approximately 15% of the patients experience a severe for of the illness that often requires assisted ventilation and oxygenation. Currently, the severity and fatality rate of COVID-19 is milder than that of SARS-CoV-1 and MERS but shows great efficiency with respect to infectivity. With similar clinical presentations as SARS-CoV-1 and MERS, the most common symptoms of COVID-19 are fever, fatigue, and respiratory symptoms, including cough, sore throat, and shortness of breath. A study of 41 hospitalized patients showed that high-levels of proinflammatory cytokines were observed in the COVID-19 severe cases (Huang et al., Lancet 395:497 (2020)). These findings are in line with SARS and MERS in that the presence of lymphopenia and “cytokine storm” likely plays a major role in the pathogenesis of COVID-19 (see, e.g., Nicholls et al., Lancet 361(9371):1773 (2003); Mahallawi et al., Cytokine 104:8 (2018); and Wong et al., Clin Exp Immunol. 136(1):95 (2004)). This so-called “cytokine storm” can initiate viral sepsis and inflammatory-induced lung injury, which can lead to other complications, including pneumonia, acute respiratory distress syndrome (ARDS), respiratory failure, septic shock, organ failure, and death. As a result, there is an urgent need for safe and effective methods of producing an immune response against coronavirus infections, such SARS-CoV-2 and related viruses.

### SUMMARY OF THE INVENTION

Disclosed herein are CpG-amphiphiles and coronavirus antigens (e.g., a coronavirus spike protein or a peptide thereof, and/or a coronavirus nucleocapsid protein or a peptide thereof, or a nucleic acid sequence encoding the same) for use in inducing an immune response in a subject. Also, disclosed are methods of administering CpG-amphiphiles and coronavirus antigens (e.g., a coronavirus spike protein or a peptide thereof, and/or a coronavirus nucleocapsid protein or a peptide thereof, or a nucleic acid sequence encoding the same) to induce an immune response in a subject.

In an aspect, the disclosure provides a method of inducing an immune response against a coronavirus antigen in a subject including administering (1) a CpG-amphiphile and (2) a coronavirus antigen or a nucleic acid sequence encoding the coronavirus antigen to the subject. Corresponding compositions and kits are also provided.

In some embodiments, the coronavirus antigen is a coronavirus spike protein or a peptide thereof or a nucleic acid sequence encoding the coronavirus spike protein or peptide. In some embodiments, the CpG-amphiphile includes a CpG sequence bonded to a lipid. In some embodiments, the CpG-amphiphile includes a CpG sequence linked to a lipid by a linker. In some embodiments, the linker includes a polymer, a string of amino acids, a string of nucleic acids, a polysaccharide, or a combination thereof. In some embodiments, the linker includes a string of nucleic acids. In some embodiments, the string of nucleic acids includes between 1 and 50 (e.g., 2, 3, 4, 5, 10, 15, 20, 25, 30, 35, 40, 45, or 50) nucleic acid residues. In some embodiments, the string of nucleic acids includes between 5 and 30 (e.g., 6, 7, 8, 9, 10, 15, 20, 25, 26, 27, 28, 29, or 30) nucleic acid residues. In some embodiments, the string of nucleic acids includes “N” guanines, where N is 1-10 (e.g., 2, 3, 4, 5, 6, 7, 8, or 9). In some embodiments, the linker includes consecutive polyethylene glycol units. In some embodiments, the linker includes “N” consecutive polyethylene glycol units, where N is between 20 and 80 (e.g., 22, 23, 24, 25, 26, 27, 28, 29, 29, 30, 35, 40, 45, 50, 55, 60, 65, 70, 75, or 80). In some embodiments, the linker includes “N” consecutive polyethylene glycol units, where N is between 30 and 70 (e.g., 31, 32, 33, 34, 35, 36, 37, 38, 39, 40, 45, 50, 55, 60, 65, or 70). In some embodiments, the linker includes “N” consecutive polyethylene glycol units, where N is between 40 and 60 (e.g., 41, 42, 43, 44, 45, 50, 55, or 60). In some embodiments, the linker includes “N” consecutive polyethylene glycol units, where N is between 45 and 55 (e.g., 46, 47, 48, 49, 50, 51, 52, 53, 54, or 55). In some embodiments, the linker includes 48 consecutive polyethylene glycol units.

In some embodiments, the lipid is a diacyl lipid. In some embodiments, the diacyl lipid has the following structure:

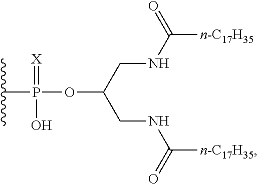

or a salt thereof, wherein X is O or S. In some embodiments, the CpG sequence includes the nucleotide sequence 5′-TCGTCGTTTTGTCGTTTTGTCGTT-3′ (SEQ ID NO:1). In some embodiments, the CpG sequence includes the nucleotide sequence of 5′-TCCATGACGTTCCTGACGTT-3′ (SEQ ID NO: 2). In some embodiments, all internucleoside groups connecting the nucleosides in the CpG sequence are phosphorothioates. In some embodiments, the coronavirus spike protein or peptide thereof is a SARS-CoV-2 spike protein or peptide thereof. In some embodiments, the peptide of the coronavirus spike protein is a receptor binding domain the specifically binds angiotensin-converting enzyme 2 (ACE2). In some embodiments, the peptide of the coronavirus spike protein including a polypeptide sequence having at least 90% (e.g., 91% 92%, 93%, 94%, 95%, 96%, 97%, 98%, or 99%) sequence identity to: RVQPTESIVRFPNITNLCPFGEVFNATRFASVYAWNRKRISNCVADYSVLYNSASFSTFKCYGVSPTK LNDLCFTNVYADSFVIRGDEVRQIAPGQTGKIADYNYKLPDDFTGCVIAWNSNNLDSKVGGNYNYLY RLFRKSNLKPFERDISTEIYQAGSTPCNGVEGFNCYFPLQSYGFQPTNGVGYQPYRVWLSFELLHA PATVCGPKKSTNLVKNKCVNFNFNGLTGTGVLTESNKKFLPFQQFGRDIADTTDAVRDPQTLEILDITP CS (SEQ ID NO: 3). In some embodiments, the peptide of the coronavirus spike protein includes the polypeptide sequence of:

(SEQ ID NO: 3)
RVQPTESIVRFPNITNLCPFGEVFNATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLS
FELLHAPATVCGPKKSTNLVKNKCVNFNFNGLTGTGVLTESNKKFLPFQ
QFGRDIADTTDAVRDPQTLEILDITPCS.

In some embodiments, the coronavirus antigen is a coronavirus nucleocapsid protein or a peptide thereof.

In some embodiments, the coronavirus nucleocapsid protein includes a polypeptide sequence having at least 90% (e.g., 91%, 92%, 93%, 94%, 95%, 96%, 97%, 98%, or 99%) sequence identity to:

(SEQ ID NO: 68)
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA.

In some embodiments, the coronavirus nucleocapsid protein includes the sequence of SEQ ID NO:68.

In some embodiments, the coronavirus nucleocapsid protein includes the polypeptide sequence of:

(SEQ ID NO: 63)
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQAENLYFQGHHHHHH.

In some embodiments, the coronavirus antigen includes one or more tags. In some embodiments, the tag is an Avi tag. In some embodiments, the tag is a histidine tag. In some embodiments, the coronavirus antigen includes an Avi tag and a histidine tag. In some embodiments, the coronavirus antigen includes a linker between the polypeptide sequence and the one or more tags. In some embodiments, the coronavirus antigen includes a protease cleavage site between the polypeptide sequence and the one or more tags. In some embodiments, the protease cleavage site is a cleavage site for a tobacco etch virus (TEV) protease (e.g., one having the sequence of ENLYFQG; SEQ ID NO:64).

In some embodiments, the coronavirus spike protein is administered. In some embodiments, a trimer of the coronavirus spike protein is administered. In some embodiments, the trimer is a trimer of a protein construct comprising a polypeptide sequence having at least 90% (e.g., 91%, 92%, 93%, 94%, 95%, 96%, 97%, 98%, or 99%) sequence identity to:

(SEQ ID NO: 66)
VNLTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTF
EYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNFRVQ
PTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQT
NSPRAAASVASQSIIAYTMSLGAENSVAYSNNSIAIPTNFTISVTTEILPV
SMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADA
GFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNE
VAKNLNESLIDLQELGKYEQYIKWPGGGSGGGSHHHHHHHHHH.

In some embodiments, the trimer includes the sequence of SEQ ID NO:66.

In some embodiments, a coronavirus spike protein, or a peptide thereof, and a coronavirus nucleocapsid protein, or a peptide thereof, are administered. In some embodiments, a trimer of a coronavirus spike protein construct comprising a polypeptide sequence having at least 90% (e.g., 91%, 92%, 93%, 94%, 95%, 96%, 97%, 98%, or 99%) sequence identity to:

(SEQ ID NO: 66)
VNLTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTF
EYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNFRVQ
PTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQT
NSPRAAASVASQSIIAYTMSLGAENSVAYSNNSIAIPTNFTISVTTEILPV
SMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADA
GFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTV
YDPLQPELDSFKEELDKYFKNHTSPDVDLGDISGINASWVNIQKEIDRLNE
VAKNLNESLIDLQELGKYEQYIKWPGGGSGGGSHHHHHHHHHH,

and a coronavirus nucleocapsid protein construct comprising a polypeptide sequence having at least 90% (e.g., 91%, 92%, 93%, 94%, 95%, 96%, 97%, 98%, or 99%) sequence identity to:
MSDNGPQNQRNAPRITFGGPSDSTGSNQNGERSGARSKQRRPQGLPNNTASWFTALTQHGKEDL KFPRGQGVPINTNSSPDDQIGYYRRATRRIRGGDGKMKDLSPRWYFYYLGTGPEAGLPYGANKDGII WVATEGALNTPKDHIGTRNPANNAAIVLQLPQGTTLPKGFYAEGSRGGSQASSRSSSRSRNSSRNS TPGSSRGTSPARMAGNGGDAALALLLLDRLNQLESKMSGKGQQQQGQTVTKKSAAEASKKPRQKR TATKAYNVTQAFGRRGPEQTQGNFGDQELIRQGTDYKHWPQIAQFAPSASAFFGMSRIGMEVTPSG TWLTYTGAIKLDDKDPNFKDQVILLNKHIDAYKTFPPTEPKKDKKKKADETQALPQRQKKQQTVTLLP AADLDDFSKQLQQSMSSADSTQAENLYFQGHHHHHH (SEQ ID NO:63) are administered.

In some embodiments, a trimer of a coronavirus spike protein construct comprising the polypeptide sequence of SEQ ID NO:66 and a coronavirus nucleocapsid protein construct comprising the polypeptide sequence of SEQ ID NO:63 are administered.

In some embodiments, an mRNA encoding the coronavirus antigen is administered. In some embodiments, the CpG-amphiphile and the coronavirus antigen or nucleic acid encoding the same are administered concurrently. In some embodiments, the CpG-amphiphile and the coronavirus antigen or nucleic acid encoding the same are administered sequentially. In some embodiments, the CpG-amphiphile is administered first, followed by administering of the coronavirus antigen or nucleic acid encoding the same. In some embodiments, the coronavirus antigen or nucleic acid encoding the same is administered first, followed by administering of CpG-amphiphile.

In some embodiments, the method comprises administering a second adjuvant to the subject.

In some embodiments the method comprises administering a coronavirus vaccine to the subject as a prime or a boost.

In some embodiments, the CpG-amphiphile is administered subcutaneously, intranasally, intratracheally, or by inhalation during mechanical ventilation. In one embodiment, the CpG-amphiphile is administered subcutaneously. In some embodiments, the coronavirus antigen (e.g., a spike protein, peptide thereof, nucleocapsid protein, or nucleic acid encoding the same) is administered subcutaneously, intranasally, intratracheally, or by inhalation during mechanical ventilation. In some embodiments, the subject is a mammal. In some embodiments, the subject is a human.

In other aspects, the disclosure provides compositions and kits that employ the components described for the above methods.

In another aspect, the disclosure provides a pharmaceutical composition comprising a CpG-amphiphile and a coronavirus antigen, or a nucleic acid sequence encoding the coronavirus antigen, and a pharmaceutically acceptable carrier. In some embodiments, the coronavirus antigen is a coronavirus spike protein or a peptide thereof. In some embodiments, the coronavirus antigen is a coronavirus nucleocapsid protein or a peptide thereof. In some embodiments, the coronavirus antigen is a combination of a coronavirus spike protein or a peptide thereof, and a coronavirus nucleocapsid protein or a peptide thereof. In some embodiments, the CpG-amphiphile is as more specifically described in the embodiments provided above and elsewhere herein and/or the coronavirus antigen is as more specifically described in the embodiments provided above and elsewhere herein.

In some embodiments, the subject is administered a dosage of about 10 μg to about 1.0 mg of the coronavirus antigen (e.g., a coronavirus spike protein or a peptide thereof, and/or a coronavirus nucleocapsid protein or peptide thereof, or a nucleic acid sequence encoding the same). In other embodiments, the dosage of the coronavirus antigen administered is about 10 μg to 1 mg, 40 μg to 60 μg, is about 50 μg to 70 μg, is about 50 μg to 150 μg, is about 70 μg to 150 μg, is about 100 μg to 150 μg, is about 100 μg to 200 μg, is about 140 μg to 250 μg, is about 200 μg to 300 μg, is about 250 μg to 500 μg, is about 300 μg to 600 μg, or is about 500 μg to 1.0 mg. In other embodiments, the dosage of the coronavirus antigen administered to the subject is about 10 μg, 20 μg, 30 μg, 40 μg, 50 μg, 60, μg, 70 μg, 80 μg, 90 μg, 100 μg, 110 μg, 120 μg, 130 μg, 140 μg, 150 μg, 200 μg, 250 μg, 300 μg, 400 μg, 500 μg, 600 μg, 700 μg, 800 μg, 900 μg, or 1.0 mg.

In some embodiments, the subject is administered a dosage of the CpG amphiphile of about 0.1 mg to 20 mg. In other embodiments, the dosage of the CpG amphiphile administered is about 0.1 mg to 1.0 mg, is about 0.5 mg to 3.0 mg, is about 1.0 mg to about 5.0 mg, is about 2.0 to 5.0 mg, is about 3.0 to 5.0 mg, is about 3.0 mg to about 10.0 mg, is about 4.0 mg to 12.0 mg, is about 5.0 mg to 15.0 mg, or is about 50 mg to 20.0 mg. The other embodiments, the dosage of the CpG amphiphile administered to the subject is about 0.1 mg, 0.2 mg, 0.3 mg, 0.4 mg, 0.5 mg, 1.0 mg, 2.0 mg, 3.0 mg, 4.0 mg, 5.0 mg, 6.0 mg, 7.0 mg, 8.0 mg, 9.0 mg, 10.0 mg, 11.0 mg, 12.0 mg, 13.0 mg, 14.0 mg, 15.0 mg, 16.0 mg, 17.0 mg, 18.0 mg, 19.0 mg, or 20.0 mg.

In another aspect, the disclosure provides a kit comprising a CpG-amphiphile and a coronavirus antigen or a nucleic acid sequence encoding the coronavirus antigen. In some embodiments, the coronavirus antigen is a coronavirus spike protein or a peptide thereof. In some embodiments, the coronavirus antigen is a coronavirus nucleocapsid protein or a peptide thereof. In some embodiments, the coronavirus antigen is a combination of a coronavirus spike protein or a peptide thereof, and a coronavirus nucleocapsid protein or a peptide thereof.

### BRIEF DESCRIPTION OF THE DRAWINGS

FIG. 1A-FIG. 1C are graphs showing the amount of serum IgG/IgM antibodies measured by an enzyme-linked immunosorbent assay (ELISA) assay for C57Bl6 mice which were administered two doses of 10 μg of a coronavirus spike protein (SEQ ID NO: 3) and 8 μg of either soluble CpG (FIG. 1A) or a CpG-amphiphile (FIG. 1B) over a range of dilutions for (from left to right) mice that were administered PBS (Mock), coronavirus spike protein with soluble CpG (Soluble CpG), or coronavirus spike protein with AMP-CpG (AMP-CpG) (FIG. 1C).

FIG. 2A-FIG. 2C are graphs showing the amount of serum IgG/IgM antibodies measured by an ELISA assay for C57Bl6 mice which were administered three doses of 10 μg of a coronavirus spike protein (SEQ ID NO: 3) and 8 μg of either soluble CpG or a CpG-amphiphile. FIG. 2A is a graph showing the OD450 for serum from mice who were administered the soluble CpG; FIG. 2B is a graph showing the OD450 for serum from mice who were administered the CpG-amphiphile; and FIG. 2C is a graph showing the amount of IgG/M titer for mice that were administered (from left to right) PBS as a control (Mock), coronavirus spike protein with soluble CpG (Soluble CpG), or coronavirus spike protein with AMP-CpG (AMP-CpG).

FIG. 3A-FIG. 3D are graphs showing the concentration of neutralizing antibodies produced that block the ability of the coronavirus spike protein to interact with the angiotensin-converting enzyme 2 (ACE2) receptor for C57Bl6 mice that were administered three doses of 10 μg of a coronavirus spike protein (SEQ ID NO: 3) and 8 μg of either soluble CpG (FIG. 3A) or a CpG-amphiphile (FIG. 3B) in comparison to human convalescent serum (FIG. 3C). FIG. 3D shows the amount of neutralization antibodies produced for (from left to right) mice that were administered PBS as a control (Mock), coronavirus spike protein with soluble CpG (Soluble CpG), or coronavirus spike protein with AMP-CpG (AMP-CpG), compared to human convalescent serum.

FIG. 4A-FIG. 4C are graphs showing the amount of IFNγ (also referred to as IFNg) (FIG. 4A), TNFa (also referred to as TNFα) (FIG. 4B), and IL6 (FIG. 4C) produced by C57Bl6 mice who were administered three doses of (from left to right) PBS as a control (Mock), 10 μg of a coronavirus spike protein (SEQ ID NO: 3) and 8 μg of soluble CpG (Soluble CpG), or 10 μg of a coronavirus spike protein (SEQ ID NO: 3) and 8 μg of CpG-amphiphile (AMP-CpG).

FIG. 5A and FIG. 5B are graphs showing the concentration of IFNg produced in C57Bl6 mice (FIG. 5A) and Balb/C mice (FIG. 5B) that were administered three doses of (from left to right) PBS as a control (Mock), 10 μg of a coronavirus spike protein (SEQ ID NO: 3) and 8 μg of soluble CpG (Soluble CpG), 10 μg of a coronavirus spike protein (SEQ ID NO: 3) and 8 μg of CpG-amphiphile (AMP-CpG).

FIG. 6A-FIG. 6C are graphs showing the amount of serum IgG/IgM antibodies measured by an ELISA assay for Balb/C mice which were administered two doses of 10 μg of a coronavirus spike protein (SEQ ID NO: 3) and 8 μg of either soluble CpG (FIG. 6A) or a CpG-amphiphile (FIG. 6B) over a range of dilutions for mice that were administered (from left to right) a PBS control (Mock), coronavirus spike protein with soluble CpG (Soluble CpG), or coronavirus spike protein with AMP-CpG (AMP-CpG) (FIG. 6C).

FIG. 7A-FIG. 7C are graphs showing the amount of serum IgG/IgM antibodies measured by an ELISA assay for Balb/C mice which were administered three doses of 10 μg of a coronavirus spike protein (SEQ ID NO: 3) and 8 μg of either soluble CpG or a CpG-amphiphile. FIG. 7A is a graph showing the OD450 for serum from mice who were administered the soluble CpG; FIG. 7B is a graph showing the OD450 for serum from mice who were administered the CpG-amphiphile; and FIG. 7C is a graph showing the amount of IgG/M titer for mice that were administered (from left to right) a PBS control (Mock), coronavirus spike protein with soluble CpG (Soluble CpG), or coronavirus spike protein with AMP-CpG (AMP-CpG).

FIG. 8A-FIG. 8D are graphs showing the concentration of neutralizing antibodies produced that block the ability of the coronavirus spike protein to interact with the ACE2 receptor for Balb/C mice that were administered three doses of 10 μg of a coronavirus spike protein (SEQ ID NO: 3) and 8 μg of either soluble CpG (FIG. 8A) or a CpG-amphiphile (FIG. 8B) in comparison to human convalescent serum (FIG. 8C). FIG. 8D shows the amount of neutralization antibodies produced for mice that were administered (from left to right) a PBS control (Mock), coronavirus spike protein with soluble CpG (Soluble CpG), or coronavirus spike protein with AMP-CpG (AMP-CpG), in comparison to human convalescent serum.

FIG. 9A-FIG. 9C are graphs showing the amount of IFNγ (FIG. 9A), TNFa (FIG. 9B), and IL6 (FIG. 9C) produced by Balb/C mice which were administered three doses (from left to right) a PBS control (Mock), 10 μg of a coronavirus spike protein (SEQ ID NO: 3) and 8 μg of soluble CpG (Soluble CpG), or 10 μg of a coronavirus spike protein (SEQ ID NO: 3) and 8 μg of CpG-amphiphile (AMP-CpG).

FIG. 10 is a graph showing the amount of (from left to right for each column) TNFa, IFNg, IL-6, IL-2, and IL-4 produced in mice which were administered two doses of 10 μg of a coronavirus spike protein (SEQ ID NO: 3) and 8 μg of either soluble CpG or a CpG-amphiphile in comparison to mice that were administered alum, IFA, or a control.

FIG. 11 is a graph showing the splenocyte IFNγ co-culture ELISpot responses of C57Bl6 mice and Balb/C mice that were administered four doses of 10 μg of a coronavirus spike protein (SEQ ID NO: 3) and 8 μg of either CpG-amphiphile, soluble CpG, or a Mock Tx in comparison to a positive or negative control.

FIG. 12A-FIG. 12D are graphs showing the amount of IgG1 (FIG. 12A), IgG2bc (FIG. 12B), IgG3 (FIG. 12C), and the IgG2bc:IgG1 ratio (FIG. 12D) for C57Bl6 mice administered three doses of (from left to right) a PBS control (Mock), Alum, IFA, 10 μg of a coronavirus spike protein (SEQ ID NO: 3) and 8 μg soluble CpG (Soluble CpG), or 10 μg of a coronavirus spike protein (SEQ ID NO: 3) and 8 μg CpG-amphiphile (AMP-CpG). The ratio of IgG2bc:IgG1 in FIG. 12D shows that for, Amp-CpG, the immune response skews strongly to Th1 and not Th2. A Th2 response can be detrimental for SARS-CoV-2.

FIG. 13A-FIG. 13D are graphs showing the amount of IFNγ (FIG. 13A), TNFa (FIG. 13B), IL-2 (FIG. 13C), and IL-6 (FIG. 13D produced by mice which were administered two doses of 10 μg of a coronavirus spike protein (SEQ ID NO: 3) and 8 μg of either CpG-amphiphile, soluble CpG, Alhydrogel, IFA, or Mock Tx in comparison to a positive or negative control.

FIG. 14A-FIG. 14D are graphs showing the amount of IFNγ (FIG. 14A), TNFa (FIG. 14B), IL-2 (FIG. 14C), and IL-6 (FIG. 14D) produced by mice which were administered three doses of 10 μg of a coronavirus spike protein (SEQ ID NO: 3) and 8 μg of either CpG-amphiphile, soluble CpG, Alhydrogel, IFA, or Mock Tx in comparison to a positive or negative control.

FIG. 15 is a graph showing the percent of (from top to bottom in each column) both IFNγ and TNFα, only TNFα, and only IFNγ in CD8 T-cells in mice that were administered three doses of 10 μg of a coronavirus spike protein (SEQ ID NO: 3) and 8 μg of either CpG-amphiphile, soluble CpG, Alhydrogel, IFA, or Mock Tx in comparison to a positive or negative control.

FIG. 16A-FIG. 16B are graphs showing the amount of pseudovirus neutralization titer at half maximal inhibitory dilution (pVNT₅₀) in C57Bl/6J mice (FIG. 16A) and BALB/c mice (FIG. 16B) (n=5 per group) that were administered four doses of 10 μg of a coronavirus spike protein (SEQ ID NO: 3) in combination 1 nmol soluble CpG or AMP-CpG compared to convalescent serum. Values depicted are mean±standard deviation. Not detected values are shown on the baseline; *P<0.05; ** P<0.01; *** P<0.001; **** P<0.0001, ns=not significant by two-sided Mann-Whitney test. Pseudovirus LOD (indicated by the dotted line) was determined as mean +90% CI calculated for mock treatment.

FIG. 16C-FIG. 16D are graphs showing the amount of IFNγ produced by either C57Bl/6J mice (FIG. 16C) or BALB/c mice (FIG. 16D) (n=5 per group) that had been administered four doses of 10 μg of a coronavirus spike protein (SEQ ID NO: 3) in combination with 1 nmol soluble CpG or AMP-CpG. Values depicted are mean±standard deviation. Not detected values are shown on the baseline; * P<0.05; ** P<0.01; *** P<0.001; **** P<0.0001; ns=not significant, by two-sided Mann-Whitney test. Pseudovirus LOD (indicated by the dotted line) was determined as mean +90% CI calculated for mock treatment.

FIG. 17A: is a graph showing the number of IFNγ spot forming cells per 1×10⁶ splenocytes that were restimulated with overlapping coronavirus spike peptides in C57BL/6J mice (n=10 per group) that received three doses of 10 μg of a coronavirus spike protein (SEQ ID NO: 3) in combination with 100 μg Alum, 1 nmol soluble CpG, or 1 nmol AMP-CpG. Values depicted are mean±standard deviation. *** P<0.001; **** P<0.0001, by two-sided Mann-Whitney test applied to cytokine⁺ T cell frequencies.

FIG. 17B-FIG. 17C are graphs showing the frequency of intracellular cytokine production, including, from top to bottom in each column, IFNγ and TNFα, only TNFα, and only IFNγ, in CD8⁺ T cells (FIG. 17B) or CD4⁺ T cells (FIG. 17C) isolated from peripheral blood cells that were restimulated with overlapping coronavirus spike peptides in C57BL/6J mice (n=10 per group) that were administered three doses of 10 μg of a coronavirus spike protein (SEQ ID NO: 3) in combination with 100 μg Alum, 1 nmol soluble CpG, or 1 nmol AMP-CpG. Values depicted are mean±standard deviation. * P<0.05; ** P<0.01; *** P<0.001; **** P<0.0001; ns=not significant, by two-sided Mann-Whitney test applied to cytokine⁺ T cell frequencies.

FIG. 18A-FIG. 18B are graphs showing the frequency of intracellular cytokine production, including, from top to bottom in each column, IFNγ and TNFα, only TNFα, and only IFNγ, in CD8⁺ T cells (FIG. 18A) or CD4⁺ T cells (FIG. 18B) isolated from perfuse lung tissue that was restimulated with overlapping coronavirus spike peptides in C57BL/6J mice (n=10 per group) that were administered three doses of 10 μg of a coronavirus spike protein (SEQ ID NO: 3) in combination with 100 μg Alum, 1 nmol soluble CpG, or 1 nmol AMP-CpG. Values depicted are mean±standard deviation. * P<0.05; ** P<0.01; *** P<0.001; **** P<0.0001, ns=not significant, by two-sided Mann-Whitney test applied to cytokine⁺ T cell frequencies or cytokine concentrations.

FIG. 18C-FIG. 18D are graphs showing the cytokine concentration, including IFNγ (FIG. 18C), TFNα, IL-6, IL-4, IL-10, and IL17 (FIG. 18D), found in the supernatants of perfuse lung tissue that was restimulated with overlapping coronavirus spike peptides in C57BL/6J mice (n=10 per group) that were administered three doses of 10 μg of a coronavirus spike protein (SEQ ID NO: 3) in combination with 100 μg Alum, 1 nmol soluble CpG, or 1 nmol AMP-CpG. Values depicted are mean±standard deviation. * P<0.05; ** P<0.01; *** P<0.001; **** P<0.0001, ns=not significant, by two-sided Mann-Whitney test applied to cytokine⁺ T cell frequencies or cytokine concentrations.

FIG. 19A-FIG. 19F are graphs showing the CD8⁺ (FIG. 19A) and the CD4⁺ (FIG. 19D) T cell count, the percentage of naive CD8⁺ (FIG. 19B) and naive CD4⁺ (FIG. 19E) T-cells, and the percent of effector memory CD8⁺ (FIG. 19C) and CD4⁺ (FIG. 19F) T-cells in cells collected from bronchoalveolar lavage in C57BL/6J mice (n=10 per group) that were administered three doses of 10 μg of a coronavirus spike protein (SEQ ID NO: 3) in combination with (from left to right) 100 μg Alum, 1 nmol soluble CpG, or 1 nmol AMP-CpG. Values depicted are mean±standard deviation. * P<0.05; ** P<0.01; *** P<0.001; **** P<0.0001; ns=not significant, by two-sided Mann-Whitney test applied to T cell frequencies.

FIG. 20A-FIG. 20G are graphs showing the humoral responses of C57Bl/6J mice (n=10 per group) that were administered three doses of 10 μg of a coronavirus spike protein (SEQ ID NO: 3) in combination with 100 μg Alum, 1 nmol soluble CpG, or 1 nmol AMP-CpG. The humoral response was assessed in serum for neutralization titer in comparison to convalescent serum (FIG. 20A), IgM (FIG. 20B), IgG (FIG. 20C), IgG1 (FIG. 20D), IgG2bc (FIG. 20E), the ratio of IgG2bc to IgG19 (FIG. 20F), and IgG3 (FIG. 20G) using either a pseudovirus neutralization assay or ELISA assay. Values depicted are mean±standard deviation. Not detected values are shown on the baseline; * P<0.05; ** P<0.01; *** P<0.001; **** P<0.0001, ns=not significant, by two-sided Mann-Whitney test.

FIG. 21A is a graph showing frequency of IFNγ spot forming cells per 1×10⁶ splenocytes in splenocytes that were restimulated with overlapping coronavirus spike peptides from C57BL/6J mice (n=10 per group) that were administered three doses of only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3). Values depicted are mean±standard deviation. Not detected values are shown on the baseline; *P<0.05; ** P<0.01; *** P<0.001; **** P<0.0001; ns=not significant, by two-sided Mann-Whitney test applied to cytokine⁺ T cell frequencies.

FIG. 21B-FIG. 21C are graphs showing frequency cytokines, including (from top to bottom in each column) IFNγ and TNFα, only TNFα, and only IFNγ, of CD8⁺ T-cells (FIG. 21B) and CD4⁺ T-cells (FIG. 21C) found in peripheral blood cells collected from C57BL/6J mice (n=10 per group) that were administered three doses of only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3). Values depicted are mean±standard deviation. Not detected values are shown on the baseline; *P<0.05; ** P<0.01; *** P<0.001; **** P<0.0001; ns=not significant, by two-sided Mann-Whitney test applied to cytokine⁺ T cell frequencies.

FIG. 21D-FIG. 21E are graphs showing frequency of cytokines, including (from top to bottom in each column) IFNγ and TNFα, only TNFα, and only IFNγ, of CD8⁺ T-cells (FIG. 21D) and CD4⁺ (FIG. 21E) found in perfused lung tissue cells, restimulated with overlapping coronavirus spike peptides, collected from C57BL/6J mice (n=10 per group) that were administered three doses of only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3). Values depicted are mean±standard deviation. Not detected values are shown on the baseline; *P<0.05; ** P<0.01; *** P<0.001; **** P<0.0001; ns=not significant, by two-sided Mann-Whitney test applied to cytokine⁺ T cell frequencies.

FIG. 22A-FIG. 22G are graphs showing the humoral responses assessed in serum for neutralization titer in comparison to convalescent serum (FIG. 22A), IgM (FIG. 22B), IgG (FIG. 22C), IgG1 (FIG. 22D), IgG2bc (FIG. 22E), the ratio of IgG2bc to IgG19 (FIG. 22F), and IgG3 (FIG. 22G) using either a pseudovirus neutralization assay or ELISA assay for C57Bl/6J mice (n=10 per group) that were administered three doses of only 10 μg of a coronavirus spike protein (SEQ ID NO: 3) in combination with only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3). Values depicted are mean±standard deviation. Not detected values are shown on the baseline; *P<0.05; ** P<0.01; *** P<0.001; **** P<0.0001 by two-sided Mann-Whitney test.

FIG. 23A-FIG. 23B are graphs showing the frequency of cytokines, including (from top to bottom in each column) IFNγ and TNFα, only TNFα, and only IFNγ, found in peripheral blood cells collected from 37 week old C57BL/6J mice (n=10 per group) that were administered three doses of only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), and 1 nmol AMP-CpG and 10 μg of a coronavirus spike protein (SEQ ID NO: 3) (FIG. 23A); and in C57BL/6J mice that were administered three doses of (from left to right) 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3) (FIG. 23B). Values depicted are mean±standard deviation. * P<0.05; ** P<0.01; *** P<0.001; **** P<0.0001; ns=not significant by two-sided Mann-Whitney test applied to cytokine⁺ T cell frequencies.

FIG. 23C-FIG. 23D are graphs showing the frequency of cytokines, including (from top to bottom in each column) IFNγ and TNFα, only TNFα, and only IFNγ, found in perfused lung tissue cells collected from 37 week old C57BL/6J mice (n=10 per group) that were administered three doses of (from left to right) only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), and 1 nmol AMP-CpG and 10 μg of a coronavirus spike protein (SEQ ID NO: 3) (FIG. 23C); and in C57BL/6J mice that were administered three doses of (from left to right) 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3) (FIG. 23D). Values depicted are mean±standard deviation. ** P<0.01; **** P<0.0001; ns=not significant by two-sided Mann-Whitney test applied to cytokine⁺ T cell frequencies.

FIG. 23E-FIG. 23F are graphs showing the frequency of cytokines, including (from top to bottom) IFNγ and TNFα, only TNFα, and only IFNγ, found in perfused lung tissue cells, restimulated with overlapping coronavirus spike peptides, that were collected from 37 week old C57BL/6J mice (n=per group) that were administered three doses of (from left to right) only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), and 1 nmol AMP-CpG and 10 μg of a coronavirus spike protein (SEQ ID NO: 3) (FIG. 23A); and in C57BL/6J mice that were administered three doses of (from left to right) 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3) (FIG. 23F). Values depicted are mean±standard deviation. *P<0.05; ** P<0.01; *** P<0.001; **** P<0.0001; ns=not significant by two-sided Mann-Whitney test applied to cytokine⁺ T cell frequencies.

FIG. 24A is a graph showing the amount of pseudovirus neutralization titer at half maximal inhibitory dilution (pVNT₅₀) in 37 week old C57Bl/6J mice (n=10 per group) that were administered three doses of only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), and 1 nmol AMP-CpG and 10 μg of a coronavirus spike protein (SEQ ID NO: 3), compared to convalescent serum. Values depicted are mean±standard deviation. Not detected values are shown on the baseline; *P<0.05; ** P<0.01; *** P<0.001 by two-sided Mann-Whitney test.

FIG. 24B is a graph showing the amount of pseudovirus neutralization titer at half maximal inhibitory dilution (pVNT₅₀) in 37 week old C57Bl/6J mice (n=10 per group) that were administered three doses of 100 μg Alum and 10 μg of a coronavirus spike protein (SEQ ID NO: 3), 1 nmol soluble CpG and 10 μg of a coronavirus spike protein (SEQ ID NO: 3), and 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3), compared to convalescent serum. Values depicted are mean±standard deviation. Not detected values are shown on the baseline; * P<0.05; ** P<0.01; ns=not significant by two-sided Mann-Whitney test.

FIG. 24C-FIG. 24G are graphs showing the humoral responses of 37 week old C57Bl/6J mice (n=10 per group) that were administered three doses of only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), and 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3). The humoral response was assessed in serum for IgG (FIG. 24C), IgG1 (FIG. 24D), IgG2bc (FIG. 24E), the ratio of IgG2bc to IgG19 (FIG. 24F), and IgG3 (FIG. 24G) using either a pseudovirus neutralization assay or ELISA assay. Values depicted are mean±standard deviation. Not detected values are shown on the baseline; * P<0.05; ** P<0.01; *** P<0.001; **** P<0.0001, ns=not significant by two-sided Mann-Whitney test.

FIG. 25A-FIG. 25D are a series of graphs showing that vaccination with AMP-CpG in aged mice enables durable Spike RBD-specific T cells in blood, spleen, and lung tissue. 37 week old C57Bl/6 mice (n=5-10 per group) were immunized on day 0, 14, and 28 with 10 ug Spike RBD protein admixed with 100 ug Alum or 1 nmol soluble CpG, or AMP-CpG. Adjuvant control animals were dosed with AMP-CpG adjuvant alone. Humoral responses specific to Spike RBD were assessed in serum from immunized animals by ELISA on day 35, 49, and 70. Shown are endpoint titers determined for IgG (FIG. 25A). T cell responses were analyzed on day 21, 35, 49, and 70. Cells were collected from peripheral blood on day 21, 35, 49, and 70 (FIG. 25B) and were restimulated with overlapping Spike RBD peptides and assayed for intracellular cytokine production to detect antigen-specific T cell responses. Shown are frequencies of IFNγ-positive cells among peripheral blood CD8⁺ T cells (FIG. 25A), and cells were collected from spleen (FIG. 25C) and lungs (FIG. 25D), and were restimulated with overlapping Spike RBD peptides and assayed for IFNγ production by ELISPOT assay. Shown is the frequency of IFNγ spot forming cells (SFC) per 1×10⁶ cells (n=5 mice per group). Values depicted are mean±standard deviation. *P<0.05; ** P<0.01; *** P<0.001; **** P<0.0001 by two-sided Mann-Whitney test applied to cytokine+ T cell frequencies. In FIG. 25A, the data for day 35, 49, and 70 are shown left to right for (1) alum, (2) soluble CpG, and (3) AMP-CpG. In FIG. 25B, the data in the graph are shown bottom to top, (1) alum, (2) soluble CpG, and (3) AMP-CpG. In FIG. 25C and FIG. 25D, the data are shown left to right, (1) adjuvant control, (2) alum, (3) soluble CpG, and (4) AMP-CpG.

FIG. 26A-FIG. 26E are a series of graphs showing that two-dose vaccination with AMP-CpG-7909 elicits potent Spike RBD-specific cellular immunity in blood and lung, and humoral immunity in blood. C57Bl/6 mice (n=5 per group) were immunized on day 0 and 14 with 0.5, 1.0, or 5.0 ug Spike RBD protein admixed with 1.0, 2.5, or 5.0 nmol AMP-CpG, and T cell and IgG responses analyzed on day 21. Peripheral blood cells (FIG. 26A and FIG. 26B) or cells collected from perfused lungs (FIG. 26C and FIG. 26D) were restimulated with overlapping Spike RBD peptides and assayed by flow cytometry for intracellular cytokine production to detect antigen-specific T cell responses. Shown are frequencies of IFNγ, TNFα, and double-positive T cells among CD8⁺ (FIG. 26A and FIG. 26C) and CD4⁺ (FIG. 26B and FIG. 26D) T cells. Humoral responses specific to Spike RBD were assessed in serum from immunized animals by ELISA. Shown are endpoint titers for IgG on day 35 (n=5 mice per group; FIG. 26E). Values depicted are mean±standard deviation. In FIG. 26A-FIG. 26D, for each bar, INFγ⁺ and TNFα⁺ are at the top of the bar, TNFα⁺ is at the middle of the bar, and INFγ⁺ is at the bottom of the bar.

FIG. 27 is a series of graphs showing that AMP-CpG induces a potent polyfunctional CD8 T cell response targeting SARS CoV-2 spike protein. A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0%), alum (0%), soluble CpG (5%), and AMP-CpG (34%). In the bar graph showing percent cytokine positive of CD8⁺ T cells, the top of each bar shows IFNγ⁺ and TNFα⁺, the middle of the bar shows TNF α⁺, and the bottom of the bar shows IFNγ⁺ cells.

FIG. 28 is a series of graphs showing that AMP-CpG induces a potent polyfunctional CD4 T cell response targeting SARS CoV-2 spike protein. A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0.2%), alum (0.5%), soluble CpG (0.5%), and AMP-CpG (12%). In the bar graph showing percent cytokine positive of CD4⁺ T cells, the top of each bar shows IFNγ⁺ and TNFα⁺, the middle of the bar shows TNF α⁺, and the bottom of the bar shows IFNγ⁺ cells.

FIG. 29 is a graph showing the number of IFNγ spot forming cells per 1×10⁶ splenocytes that were restimulated with overlapping coronavirus spike peptides in C57BL/6J mice (n=10 per group) that had received a mock vaccine or 10 μg of a full-length coronavirus spike protein construct (SEQ ID NO: 66) in combination with 10 μg of a coronavirus nucleocapsid protein construct (SEQ ID NO:63) and (1) 100 μg alum, (2) 6 μg soluble CpG, or (3) 6 μg AMP-CpG. Values depicted are mean±standard deviation. This graph shows that AMP-CpG induces a potent T cell response targeting SARS CoV-2 spike protein.

FIG. 30 is a series of graphs showing that AMP-CpG induces a potent lung-resident polyfunctional CD8⁺ T cell response targeting SARS CoV-2 spike protein. A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0%), alum (0%), soluble CpG (3%), and AMP-CpG (26%). In the bar graph showing percent cytokine positive of CD8⁺ T cells, the top of each bar shows IFNγ⁺ and TNFα⁺, the middle of the bar shows TNFα⁺, and the bottom of the bar shows IFNγ⁺ cells.

FIG. 31 is a series of graphs showing that AMP-CpG induces a potent lung-resident polyfunctional CD4⁺ T cell response targeting SARS CoV-2 spike protein. A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0.2%), alum (0.2%), soluble CpG (1%), and AMP-CpG (7%). In the bar graph showing percent cytokine positive of CD4⁺ T cells, the top of each bar shows IFNγ⁺ and TNFα⁺, the middle of the bar shows TNFα⁺, and the bottom of the bar shows IFNγ⁺ cells.

FIG. 32 is a series of graphs showing that AMP-CpG induces a potent peripheral blood polyfunctional CD8⁺ and CD4⁺ T cell response targeting SARS CoV-2 nucleocapsid protein. A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. In the bar graphs showing percent cytokine positive of CD8⁺ T cells or percent cytokine positive of CD4⁺ T cells, the top of each bar shows IFNγ⁺ and TNFα⁺, the middle of the bar shows TNF α⁺, and the bottom of the bar shows IFNγ⁺ cells.

FIG. 33 is a graph showing the number of IFNγ spot forming cells per 1×10⁶ splenocytes that were restimulated with overlapping coronavirus nucleocapsid peptides in C57BL/6J mice (n=10 per group) that received a mock vaccine or 10 μg of a full-length coronavirus spike protein construct (SEQ ID NO: 66) in combination with 10 μg of a coronavirus nucleocapsid protein construct (SEQ ID NO:63) and (1) 100 μg alum, (2) 6 μg soluble CpG, or (3) 6 μg AMP-CpG. Values depicted are mean±standard deviation. This graph shows that AMP-CpG induces a potent T cell response targeting SARS CoV-2 nucleocapsid protein.

FIG. 34 is a graph showing that the reformulated AMP-CpG vaccine induced a robust antibody response to Genscript RBD in non-human primates. The dotted line indicates the assay limit of detection (LOD).

FIG. 35 is a graph showing that the reformulated AMP-CpG vaccine induces IgG antibodies to the UK SARS-CoV-2 variant (right column). Wild-type SARS-CoV-2 is shown in the left column. The dotted line indicates the assay LOD.

FIG. 36A and FIG. 36B are graphs showing that the reformulated AMP-CPG vaccine induces CD8⁺ T-cell responses to spike RBD.

FIG. 37A and FIG. 37B are graphs showing that the reformulated AMP-CPG vaccine induces CD4⁺ and CD8⁺ T-cell responses to spike RBD. In each column, % TNFα is shown at the top, % IL2 is shown in the middle, and % IFNγ is shown at the bottom.

FIG. 38 is a graph showing results of a tetramer analysis for C57BL/6J mice administered two doses of adjuvant control (Adj only), reformulated AMP-CPG dual WT RBD and B.1.351 RBD vaccine having 5 mg per 100 μL injection of each WT RBD and B.1.351 RBD antigens (Dual Vax), or reformulated AMP-CPG B.1.351 RBD vaccine having 5 mg per 100 μL injection of B.1.351 RBD antigen (Amp Vax).

FIG. 39A, FIG. 39B, and FIG. 39C are graphs showing results of an Intracellular Stain (ICS) analysis for C57BL/6J mice administered two doses of adjuvant control (Adj only), reformulated AMP-CPG dual WT RBD and B.1.351 RBD vaccine having 5 mg per 100 μL injection of each WT RBD and B.1.351 RBD antigens (Dual Vax), or reformulated AMP-CPG B.1.351 vaccine having 5 mg per 100 μL injection of B.1.351 antigen (B.1.351). FIG. 39A shows that the reformulated AMP-CPG dual WT RBD and B.1.351 RBD vaccine and the reformulated AMP-CPG B.1.351 vaccine induces CD8⁺ lung cells to secrete more cytokines IFNγ and TNFα as compared to the adjuvant only vaccine following dose 2.

FIG. 39B shows that the reformulated AMP-CPG dual WT RBD and B.1.351 RBD vaccine and the reformulated AMP-CPG B.1.351 vaccine induces CD4⁺ lung cells to secrete more cytokines IFNγ and TNFα as compared to the adjuvant only vaccine following dose 2. FIG. 39C shows that the reformulated AMP-CPG dual WT RBD and B.1.351 RBD vaccine and the reformulated AMP-CPG B.1.351 vaccine induces CD8⁺ blood cells to secrete more cytokines IFNγ and TNFα as compared to the adjuvant only vaccine following dose 2. In each column % IFNγ+TNFα is shown at the top, % TNFα is shown in the middle, and % IFNγ is shown at the bottom.

FIG. 40 is a graph showing results of an ELISpot analysis for C57BL/6J mice administered two doses of adjuvant control (Adj only), reformulated AMP-CPG dual WT RBD and B.1.351 RBD vaccine having 5 mg per 100 μL injection of each WT RBD and B.1.351 RBD antigens (Dual Vax), or reformulated AMP-CPG B.1.351 vaccine having 5 mg per 100 μL injection of B.1.351 RBD antigen (B.1.351).

FIG. 41 is a graph showing the amount of antibody serum measured by ELISA analysis for C57BL/6J mice administered two doses of adjuvant control (Adj only), reformulated AMP-CPG dual WT RBD and B.1.351 RBD vaccine having 5 mg per 100 μL injection of each WT RBD and B.1.351 RBD antigens (Dual Vax), or reformulated AMP-CPG B.1.351 vaccine having 5 mg per 100 μL injection of B.1.351 RBD antigen (B.1.351).

### DEFINITIONS

Terms used in the claims and specification are defined as set forth below unless otherwise specified.

It must be noted that, as used in the specification and the appended claims, the singular forms “a,” “an,” and “the” include plural referents unless the context clearly dictates otherwise.

As used herein, the term “about” refers to a value that is within 10% above or below the value being described.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. The term “amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, the terms “amphiphile” and “amphiphilic” refer to a conjugate comprising a hydrophilic head group and a hydrophobic tail, thereby forming an amphiphilic conjugate. In some embodiments, an amphiphile conjugate comprises a CpG oligodeoxynucleotide (ODN) and one or more hydrophobic lipid tails, referred to herein as a “CpG-amphiphile.”

As used herein, “conjugated” refers to covalent attachment or crosslink of the CpG-amphiphile to a lipid. The CpG-amphiphile may be bonded to the lipid through a covalent attachment by reaction of complementary reactive groups on the CpG-amphiphile and the lipid.

As used herein, the terms “CpG oligodeoxynucleotide” and “CpG motif” refer to a short single-stranded DNA molecule which includes a 5′ C nucleotide connected to a 3′ G nucleotide through a phosphodiester internucleotide linkage or a phosphodiester derivative internucleotide linkage. In some embodiments, a CpG motif includes a phosphodiester internucleotide linkage. In some embodiments, a CpG motif includes a phosphodiester derivative internucleotide linkage.

As used herein, the terms “coronavirus spike protein” and “coronavirus spike peptide” refer to a full-length or fragment of a large, type 1 transmembrane protein, sometimes referred to as an “S protein,” which includes an S1 and S2 domain. Coronavirus spike proteins are highly glycosylated and assemble in trimers on the virion surface, such as the surface of the SAR-CoV-2 virion. In the case of SARS-CoV-2, the spike protein binds with a human angiotensin-converting enzyme 2 (ACE2) receptor to infect human cells, e.g., respiratory epithelial cells (e.g., type II alveolar cells), as well as cells (e.g., epithelial cells, endothelial cells, neurons, glial cells, smooth muscle cells, and enterocytes) in many other tissues and organs including, e.g., the heart, blood vessels, kidney, liver, gastrointestinal tract, and the nervous system (e.g., the brain and the peripheral nervous system). In some embodiments, the spike protein peptide may have an amino acid sequence having at least 90% (e.g., at least 91%, 92%, 93%, 94%, 95%, 96%, 97%, 98%, or 99%) sequence identity to SEQ ID NO: 3. In some embodiments, the coronavirus spike protein peptide has an amino acid sequence of SEQ ID NO: 3.

As used herein, “immune response” refers to a response made by the immune system of an organism to a substance, which includes but is not limited to foreign or self proteins. Three general types of “immune response” include mucosal, humoral, and cellular immune responses. An immune response may include at least one of the following: antibody production, inflammation, developing immunity, developing hypersensitivity to an antigen, the response of antigen-specific lymphocytes to antigen, and transplant or graft rejection.

As used herein, “immunogenic” refers to the ability of an agent (e.g., a CpG-amphiphile and a coronavirus spike protein or peptide), to trigger an immune response, e.g., as measured by antibody titer.

As used herein, the term “immunogenic amount” refers to an amount of a CpG-amphiphile and a coronavirus spike protein or peptide that induces an immune response in a subject (e.g., reflected by an increase in antibody titer in the subject as determined by conventional techniques, such as enzyme-linked immunosorbent assay (ELISA)).

The term “infectious agent,” as used herein, refers to agents that cause an infection and/or a disease. Infectious agents include viruses, bacteria, fungi, and parasites. In some embodiments, the infectious agent is a virus (e.g., a coronavirus, e.g., SARS-CoV-2).

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

A “pharmaceutically acceptable carrier,” as used herein, refers to a vehicle capable of suspending or dissolving the active compound, and having the properties of being nontoxic and non-inflammatory in a patient. Moreover, a pharmaceutically acceptable carrier may include a pharmaceutically acceptable additive, such as a preservative, antioxidant, fragrance, emulsifier, dye, or excipient known or used in the field of drug formulation and that does not significantly interfere with the therapeutic effectiveness of the biological activity of the active agent, and that is non-toxic to the patient.

The term “pharmaceutically acceptable excipient,” as used herein, refers to any inactive ingredient having the properties of being nontoxic and non-inflammatory in a subject. Typical excipients include, for example: carriers, binders, fillers, lubricants, emulsifiers, suspending agents, sweeteners, flavorings, preservatives, buffers, wetting agents, disintegrants, effervescent agents, and other conventional excipients and additives and/or other additives that may enhance stability, delivery, absorption, half-life, efficacy, pharmacokinetics, and/or pharmacodynamics, reduce adverse side effects, or provide other advantages for pharmaceutical use.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “pharmaceutically acceptable salt,” as used herein, means any pharmaceutically acceptable salt of a conjugate, oligonucleotide, or peptide disclosed herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use (Eds. P. H. Stahl and C. G. Wermuth), Wiley-VCH, 2008. The salts can be prepared in situ during the final isolation and purification of the compounds described herein or separately by reacting a free base group with a suitable acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. References to the conjugates, oligonucleotides, or peptides include pharmaceutically acceptable salts thereof unless otherwise indicated or not applicable.

“Polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “preventing” or “reducing the risk of acquiring” means decreasing the risk of (e.g., by 1%, 2%, 3%, 4%, 5%, 6%, 7%, 8%, 9%, 10%, 20%, 30%, 40%, 50%, 60%, 70%, 80%, 90%, 95%, 97%, 99%, or about 100%) contracting an infectious disease, e.g., a viral infection, e.g., an infection by a beta-coronavirus such as SARS-CoV-2, or a related virus. To determine whether the prevention is effective, a comparison can be made between the subject who received a composition of the invention and a similarly-situated subject (e.g., one at risk of a viral infection, such as a SARS-CoV-2 infection, or an infection by a related virus) who did not receive the composition. A comparison can also be made between the subject who received the composition and a control, a baseline, or a known level of measurement.

As used here, the term “subject” or “mammal” or “patient” as used herein includes both humans and non-humans and includes, but is not limited to, humans, non-human primates, canines, felines, murines, bovines, equines, and porcines.

As used herein, the term “therapeutically effective amount” is an amount that is effective to ameliorate a symptom of a disease. A therapeutically effective amount can be a “prophylactically effective amount” as prophylaxis can be considered therapy.

The terms “treat,” “treatment,” and “treating” refer to therapeutic approaches in which the goal is to reverse, alleviate, ameliorate, inhibit, slow down, or stop the progression or severity of a condition associated with a disease or disorder, e.g., COVID-19. These terms include reducing or alleviating at least one adverse effect or symptom of a condition, disease, or disorder. Treatment is generally “effective” if one or more symptoms or clinical markers are reduced, or if a desired response (e.g., a specific immune response) is induced. Alternatively, treatment is “effective” if the progression of a disease is reduced or halted.

As used herein, the term “vaccine” or “immunogenic composition” refers to a formulation which contains a CpG-amphiphile and/or a coronavirus antigen (e.g., a coronavirus spike protein, a peptide thereof, or a nucleic acid sequence encoding the same) as described herein, optionally combined with an adjuvant, which is in a form that is capable of being administered to a vertebrate and which induces a protective or therapeutic immune response sufficient to induce immunity to prevent and/or ameliorate an infection or disease and/or to reduce at least one symptom of an infection or disease. Typically, the vaccine or immunogenic composition comprises a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine or immunogenic composition provokes an immune response including, but not limited to, the production of antibodies and/or cytokines and/or the activation of cytotoxic T cells, antigen presenting cells, helper T cells, dendritic cells and/or other cellular responses.

Other features and advantages of the invention will be apparent from the following detailed description, the drawings, and the claims.

### DETAILED DESCRIPTION OF THE INVENTION

The invention provides compositions that can be used in inducing an immune response in a subject. The compositions include CpG oligodeoxynucleotides (ODNs) linked to a lipid by way of a linker or without the use of linker (i.e., bonded directly) forming an CpG-amphiphile, and coronavirus antigen (e.g., a coronavirus spike protein or a peptide thereof, and/or a coronavirus nucleocapsid protein or a peptide thereof, or a nucleic acid sequence encoding the same). Together the compounds described herein induce an immune response in a subject, such as a human subject, when administered concurrently or separately. The CpG-amphiphile can function as an adjuvant to elicit an immune response in a subject, such as an immune response against a coronavirus antigen (e.g., a SARS-CoV-2 antigen, e.g., a SARS-CoV-2 spike protein or peptide thereof or a SARS-CoV-2 nucleocapsid protein or a peptide thereof).

### CpG

CpG oligodeoxynucleotides (ODNs) are short synthetic single-stranded DNA molecules containing unmethylated CpG dinucleotides in particular sequence contexts. CpG ODNs possess a partially or completely phosphorothioated (PS) backbone, as opposed to the natural phosphodiester (PO) backbone in DNA molecules. Three major classes of stimulatory CpG ODNs have been identified based on structural characteristics and activity on human peripheral blood mononuclear cells (PBMCs), in particular B cells and plasmacytoid dendritic cells (pDCs). These three classes are Class A (Type D), Class B (Type K), and Class C.

In some embodiments, the CpG ODN may be a Class A ODN. For example, the Class A ODN may be selected from the group including CpG 1585, having an amino acid sequence of GGGGTCAACGTTGAGGGGGG (SEQ ID NO: 5); CpG 2216, having an amino acid sequence of GGGGGACGATCGTCGGGGGG (SEQ ID NO: 6); and CpG 2336, having the amino acid sequence of GGGGACGACGTCGTGGGGGGG (SEQ ID NO: 7).

In some embodiments, the CpG ODN may be a Class B ODN. Class B CpG ODNs contain a full PS backbone with one or more CpG dinucleotides. They strongly activate B cells and TLR9-dependent NF-κB signaling but weakly stimulate IFN-α secretion. For example, the Class B ODN may be selected from the group including CpG 1668, having the amino acid sequence of TCCATGACGTTCCTGATGCT (SEQ ID NO:71): CpG 7909, also known as CpG 2006, having the amino acid sequence of TCGTCGTTTTGTCGTTTTGTCGTT (SEQ ID NO: 1); CpG 2007, having the amino acid sequence of TCGTCGTTGTCGTTTTGTCGTT (SEQ ID NO: 8); CpG BW006, having the amino acid sequence of TCGACGTTCGTCGTTCGTCGTTC (SEQ ID NO: 9); CpG D-SL01, having the amino acid sequence of TCGCGACGTTCGCCCGACGTTCGGTA (SEQ ID NO: 10); CpG 1018, having the amino acid sequence of TGACTGTGAACGTTCGAGATGA (SEQ ID NO: 15), and CpG 1826, having an amino acid sequence of TCCATGACGTTCCTGACGTT (SEQ ID NO; 2)). In some embodiments, the CpG ODN is CpG 7909 (SEQ ID NO: 1), In some embodiments, the CpG ODN is CpG 1826 (SEQ ID NO: 2).

In some embodiments, the CpG ODN may be a Class C ODN. For example, the Class C ODN may be selected from the group including CpG 2395, having the amino acid sequence of TCGTCGTTTTCGGCGCGCGCCG (SEQ ID NO: 11); CpG M362, having the amino acid sequence of TCGTCGTCGTTCGAACGACGTTGAT (SEQ ID NO: 12); and CpG D-SL03, having the amino acid sequence of TCGCGAACGTTCGCCGCGTTCGAACGCGG (SEQ ID NO: 13).

In some embodiments, all the internucleoside groups connecting the nucleosides in the CpG sequence are phosphorothionates

In some embodiments, an immunogenic composition includes an amphiphilic conjugate. An amphiphilic conjugate refers to a conjugate that includes a CpG ODN covalently linked to an albumin-binding domain (e.g., a lipid). In some embodiments, an amphiphilic conjugate includes a CpG ODN that is covalently linked to an albumin-binding domain (e.g., a lipid) directly. In some embodiments, an amphiphilic conjugate includes a CpG ODN that is covalently linked to an albumin-binding domain (e.g., a lipid) through a linker. For amphiphilic conjugates that include CpG ODN conjugated to an albumin-binding domain either directly or through a linker, the albumin binding domain binds to endogenous albumin, which prevents the CpG-amphiphile from rapidly flushing into the bloodstream and instead re-targets them to lymphatics and draining lymph nodes where they accumulate due to filtering of albumin by antigen presenting cells.

CpG ODNs may be bonded directly or linked by way of a linker to a lipid to a form an CpG amphiphile. These compounds may be produced using the ordinary phosphoramidite chemistry known in the art. In some examples, the CpG ODN or CpG ODN-GG may be reacted with the following compound: to produce an intermediate, which upon oxidation with (e.g., phosphite oxidation methods known in the art, e.g., a sulfurizing agent, such as 3-((N,N-dimethylaminomethylidene)amino)-3H-1,2,4-dithiazole-5-thione) and hydrolysis of the cyanoethyl group may produce a compound of the invention.

Reference to CpG molecules herein, as well as amphiphiles including a CpG molecule, is to be understood as including pharmaceutically acceptable salts thereof.

### Lipid

The CpG-amphiphiles disclosed herein include a hydrophobic lipid, which may be an albumin binding domain. The lipid can be linear, branched, or cyclic. The lipid is preferably at least 17 to 18 carbons in length but may be shorter if it shows good albumin binding and adequate targeting to the lymph nodes. In some embodiments, the activity relies, in-part, on the ability of the CpG-amphiphile to associate with albumin in the blood of the subject. Therefore, lymph node-targeted CpG-amphiphiles typically include a lipid that can bind to albumin under physiological conditions. Lipids suitable for targeting the lymph node can be selected based on the ability of the lipid or a lipid conjugated to a CpG ODN to bind to albumin. Suitable methods for testing the ability of the lipid or lipid conjugated to a CpG ODN to bind to albumin are known in the art.

Examples of preferred lipids for use in lymph node targeting with CpG-amphiphiles include, but are not limited to fatty acids with aliphatic tails of 8-30 carbons including, but not limited to, linear and unsaturated saturated fatty acids, branched saturated and unsaturated fatty acids, and fatty acids derivatives, such as fatty acid esters, fatty acid amides, and fatty acid thioesters, diacyl lipids, cholesterol, cholesterol derivatives, and steroid acids such as bile acids; Lipid A or combinations thereof.

In some embodiments, the lipid is a diacyl lipid or two-tailed lipid. In some embodiments, the tails in the diacyl lipid contain from about 8 to about 30 carbons and can be saturated, unsaturated, or combinations thereof. In some embodiments, the diacyl lipid has the following structure:

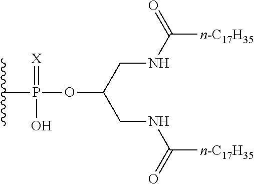

or a salt thereof, wherein X is O or S. The tails of a lipid can be coupled to the head group via ester bond linkages, amide bond linkages, thioester bond linkages, or combinations thereof. In a particular embodiment, the diacyl lipids are phosphate lipids, glycolipids, sphingolipids, or combinations thereof.

Lymph node-targeting conjugates typically include a lipid that is 8 or more carbon units in length. Increasing the number of lipid units can reduce insertion of the lipid into plasma membrane of cells, allowing the lipid conjugate to remain free to bind albumin and traffic to the lymph node. For example, the lipid can be a diacyl lipid composed of two C18 hydrocarbon tails. In some embodiments, the lipid for use in preparing lymph node targeting lipid conjugates is not a single chain hydro-carbon (e.g., C18), or cholesterol. Cholesterol conjugation has been explored to enhance the immunomodulation of molecular adjuvants such as CpG and immunogenicity of peptides.

Reference to lipids herein, as well as amphiphiles including the lipid, is to be understood as including pharmaceutically acceptable salts thereof.

### Linkers

For the CpG-amphiphile to be trafficked efficiently to the lymph node, the CpG ODN should remain soluble. Therefore, a polar block linker can be included between the CpG ODN and the lipid to which it is conjugated to increase solubility of the CpG ODN. In some embodiments, the CpG-amphiphile includes a CpG sequence linked to a lipid by a linker. The linker may reduce or prevent the ability of the lipid to insert into the plasma membrane of cells, such as cells in the tissue adjacent to the injection site. The linker can also reduce or prevent the ability of the CpG ODN from non-specifically associating with extracellular matrix proteins at the site of administration. The linker may increase the solubility of the CpG ODN without preventing its ability to bind to albumin. This combination of characteristics can allow the CpG ODN to bind to albumin present in the serum or interstitial fluid and remain in circulation until the albumin is trafficked to and retained in a lymph node.

The length and composition of the linker can be adjusted based on the lipid and CpG ODN selected. For example, for some CpG ODNs, the oligonucleotide itself may be polar enough to ensure solubility; for example, oligonucleotides that are 10, 15, 20 or more nucleotides in length. Therefore, in some embodiments, no additional linker is required. However, depending on the amino acid sequence, some lipidated peptides can be essentially insoluble. In these cases, it can be desirable to include a linker that mimics the effect of a polar oligonucleotide. A linker can be used as part of any of lipid conjugates described herein, for example, lipid-oligonucleotide conjugates and lipid-peptide conjugates, which reduce cell membrane insertion/preferential portioning onto albumin.

Suitable linkers include, but are not limited to, oligonucleotides such as those discussed above, including a string of nucleic acids, a hydrophilic polymer including but not limited to poly(ethylene glycol) (MW: 500 Da to 20,000 Da), polyacrylamide (MW: 500 Da to 20,000 Da), polyacrylic acid; a string of hydrophilic amino acids such as serine, threonine, cysteine, tyrosine, asparagine, glutamine, aspartic acid, glutamic acid, lysine, arginine, histidine, or combinations thereof; polysaccharides, including but not limited to, dextran (MW: 1,000 Da to 2,000,000 Da), or combinations thereof. The hydrophobic lipid and the linker/CpG ODN are covalently linked. The covalent bond may be a non-cleavable linkage or a cleavable linkage. The non-cleavable linkage can include an amide bond or phosphate bond, and the cleavable linkage can include a disulfide bond, acid-cleavable linkage, ester bond, anhydride bond, biodegradable bond, or enzyme-cleavable linkage.

In some embodiments, the linker is one or more ethylene glycol (EG) units, more preferably two or more EG units (i.e., polyethylene glycol (PEG)). For example, in some embodiments, the CpG-amphiphile includes a CpG and a hydrophobic lipid linked by a polyethylene glycol (PEG) molecule or a derivative or analog thereof.

In some embodiments, CpG-amphiphiles described herein contain a CpG ODN linked to PEG which is in turn linked to a hydrophobic lipid, or lipid-Gn-ON conjugates, either covalently or via formation of protein-oligo conjugates that hybridize to oligo micelles. The precise number of PEG units depends on the lipid and the cargo, however, typically, a linker can have between about 1 and about 100, between about 20 and about 80, between about 30 and about 70, or between about 40 and about 60 PEG units. In some embodiments, the linker has between about 45 and 55 PEG, units. For example, in some embodiments, the linker has 48 PEG units.

As discussed above, in some embodiments, the linker is an oligonucleotide which includes a string of nucleic acids. In some embodiments, the CpG amphiphiles described herein include a CpG ODN linked to a string of nucleic acids, which is in turn linked to a hydrophobic lipid. The linker can have any sequence, for example, the sequence of the oligonucleotide can be a random sequence, or a sequence specifically chosen for its molecular or biochemical properties (e.g., highly polar). In some embodiments, the linker includes 20 one or more series of consecutive adenine (A), cytosine (C), guanine (G), thymine (T), uracil (U), or analog thereof. In some embodiments, the linker consists of a series of consecutive adenine (A), cytosine (C), guanine (G), thymine (T), uracil (U), or analog thereof.

In some embodiments, the string of nucleic acids includes between 1 and 50 nucleic acid residues. In some embodiments, the string of nucleic acids includes between 5 and 30 nucleic acid residues. In some embodiments, the linker includes one or more guanines, for example between 1-guanines. It has been discovered that altering the number of guanines between a CpG ODN and a lipid tail controls micelle stability in the presence of serum proteins. Therefore, the number of guanines in the linker can be selected based on the desired affinity of the CpG ODN for serum proteins such as albumin.

In some embodiments, the linker is an oligonucleotide that includes a string of amino acids. In some embodiments, the CpG amphiphiles include a CpG ODN linked to string of amino acids, which is in turn linked to a hydrophobic lipid. The linker can have any amino acid sequence, for example, the sequence of the oligonucleotide can be a random sequence, or a sequence chosen for its molecular or biochemical properties (e.g., high flexibility). In some embodiments, the linker includes a series of glycine residue to form a polyglycine linker. In some embodiments, the linker includes an amino acid sequence of (Gly)ₙ, wherein n may be between 2 and 20 residues. Examples of polyglycine linkers include but are not limited to GGG, GGGA (SEQ ID NO:18), GGGG (SEQ ID NO:19), GGGAG (SEQ ID NO:20), GGGAGG (SEQ ID NO:21), GGGAGGG (SEQ ID NO:22), GGAG (SEQ ID NO:23), GGSG (SEQ ID NO:24), AGGG (SEQ ID NO:25), SGGG (SEQ ID NO:26), GGAGGA (SEQ ID NO:27), GGSGGS (SEQ ID NO:28), GGAGGAGGA (SEQ ID NO:29), GGSGGSGGS (SEQ ID NO:30), GGAGGAGGAGGA (SEQ ID NO:31), GGSGGSGGSGGS (SEQ ID NO:32), GGAGGGAG (SEQ ID NO:33), GGSGGGSG (SEQ ID NO:34), GGAGGGAGGGAG (SEQ ID NO:35), GGSGGGSGGGSG (SEQ ID NO:36), GGGGAGGGGAGGGGA (SEQ ID NO:37), GGGGSGGGGSGGGGS (SEQ ID NO:38), and GGGSGGGS (SEQ ID NO:62).

Linkers described herein (e.g., polyglycine linkers) can also be used to link a polypeptide sequence (e.g., a coronavirus spike protein or peptide thereof or a coronavirus nucleocapsid protein or a peptide thereof) to a tag (e.g., a histidine tag and/or an Avi tag).

### Coronavirus Antigen

In an aspect, the disclosure provides a full-length or fragment of a SARS-CoV-2 spike glycoprotein, which has been identified as immunogenic or a multimer (e.g., a trimer) of this spike protein (Grifoni et al. Cell Host Microbe. 2020; 27(4): 671-80; Ou et al. Nat Commun. 2020, 11(1): 1620; Walls et al. Cell. 2020; 181(2): 281-92). In addition, the antigen may correspond to SARS-CoV-2 nucleocapsid protein, membrane protein, etc., or a peptide thereof. The antigen may also correspond to a specific functional region of a coronavirus spike protein (i.e., protein subunit). For example, the antigen may correspond to or comprise the S1, S2, or receptor-binding domain (RBD) region of the SARS-CoV-2 spike glycoprotein, or S protein.

The antigen(s) may also be a peptide (or several peptides) that correspond to immunogenic sequences in the infectious agent of interest. The peptides behave as epitopes that can elicit various immune responses. For example, the peptides may represent various positions of the SARS-CoV-2 spike glycoprotein which are predicted in both cellular and humoral immunogenicity (Fast et al. bioRxiv. 2020: 2020.02.19.955484). Regarding antigens made up of several peptides, the antigen(s) may be a cocktail of overlapping peptides that encompass a whole protein or a functional region thereof, or it may be a mixture of peptides that correspond to immunogenic regions of different proteins. For example, the antigen(s) may be a mix of peptides that includes SARS-CoV-2 spike protein, nucleocapsid protein, and membrane protein The SARS-CoV-2 spike protein may have the amino acid sequence of MFIFLLFLTLTSGSDLDRCTTFDDVQAPNYTQHTSSMRGVYYPDEIFRSDTLYLTQDLFLPFYSNVTGF HTINHTFGNPVIPFKDGIYFAATEKSNVVRGWVFGSTMNNKSQSVIIINNSTNVVIRACNFELCDNPFF AVSKPMGTQTHTMIFDNAFNCTFEYISDAFSLDVSEKSGNFKHLREFVFKNKDGFLYVYKGYQPIDVV RDLPSGFNTLKPIFKLPLGINITNFRAILTAFSPAQDIWGTSAAAYFVGYLKPTTFMLKYDENGTITDAV DCSQNPLAELKCSVKSFEIDKGIYQTSNFRVVPSGDWRFPNITNLCPFGEVFNATKFPSVYAWERKK ISNCVADYSVLYNSTFFSTFKCYGVSATKLNDLCFSNVYADSFVVKGDDVRQIAPGQTGVIADYNYKL PDDFMGCVLAWNTRNIDATSTGNYNYKYRYLRHGKLRPFERDISNVPFSPDGKPCTPPALNCYWPL NDYGFYTTTGIGYQPYRWVLSFELLNAPATVCGPKLSTDLIKNQCVNFNFNGLTGTGVLTPSSKRFQ PFQQFGRDVSDFTDSVRDPKTSEILDISPCSFGGVSVITPGTNASSEVAVLYQDVNCTDVSTAIHADQ LTPAWRIYSTGNNVFQTQAGCLIGAEHVDTSYECDIPIGAGICASYHTVSLLRSTSQKSIVAYTMSLGA DSSIAYSNNTIAIPTNFSISITTEVMPVSMAKTSVDCNMYICGDSTECANLLLQYGSFCTQLNRALSGIA AEQDRNTREVFAQVKQMYKTPTLKYFGGFNFSQILPDPLKPTKRSFIEDLLFNKVTLADAGFMKQYGE CLGDINARDLICAQKFNGLTVLPPLLTDDMIAAYTAALVSGTATAGWTFGAGAALQIPFAMQMAYRFN GIGVTQNVLYENQKQIANQFNKAISQIQESLTTTSTALGKLQDWNQNAQALNTLVKQLSSNFGAISSV LNDILSRLDKVEAEVQIDRLITGRLQSLQTYVTQQLIRAAEIRASANLAATKMSECVLGQSKRVDFCGK GYHLMSFPQAAPHGVVFLHVTYVPSQERNFTTAPAICHEGKAYFPREGVFVFNGTSWFITQRNFFSP QIITTDNTFVSGNCDVVIGIINNTVYDPLQPELDSFKEELDKYFKNHTSPDVDLGDISGINASVVNIQKEI DRLNEVAKNLNESLIDLQELGKYEQYIKWPWYWVLGFIAGLIAIVMVTILLCCMTSCCSCLKGACSCGS CCKFDEDDSEPVLKGVKLHYT (SEQ ID NO: 14). In some embodiments, the coronavirus spike protein may have the amino acid sequence of

(SEQ ID NO: 16)
MFIFLLFLTLTSGSDLDRCTTFDDVQAPNYTQHTSSMRGVYYPDEIFRSDT
LYLTQDLFLPFYSNVTGFHTINHTFGNPVIPFKDGIYFAATEKSNVVRGWV
FGSTMNNKSQSVIIINNSTNVVIRACNFELCDNPFFAVSKPMGTQTHTMIF
DNAFNCTFEYISDAFSLDVSEKSGNFKHLREFVFKNKDGFLYVYKGYQPID
VVRDLPSGFNTLKPIFKLPLGINITNFRAILTAFSPAQDIWGTSAAAYFVG
YLKPTTFMLKYDENGTITDAVDCSQNPLAELKCSVKSFEIDKGIYQTSNFR
VVPSGDVVRFPNITNLCPFGEVFNATKFPSVYAWERKKISNCVADYSVLYN
STFFSTFKCYGVSATKLNDLCFSNVYADSFVVKGDDVRQIAPGQTGVIADY
NYKLPDDFMGCVLAWNTRNIDATSTGNYNYKYRYLRHGKLRPFERDISNVP
FSPDGKPCTPPALNCYWPLNDYGFYTTTGIGYQPYRVVVLSFELLNAPATV
CGPKLSTDLIKNQCVNFNFNGLTGTGVLTPSSKRFQPFQQFGRDVSDFTDS
VRDPKTSEILDISPCSFGGVSVITPGTNASSEVAVLYQDVNCTDVSTAIHA
GQLTPAWRIYSTGNNVFQTQAGCLIGAEHVDTSYECDIPIGAGICASYHTV
SLLRSTSQKSIVAYTMSLGADSSIAYSNNTIAIPTNFSISITTEVMPVSMA
KTSVDCNMYICGDSTECANLLLQYGSFCTQLNRALSGIAAEQDRNTREVFA
QVKQMYKTPTLKYFGGFNFSQILPDPLKPTKRSFIEDLLFNKVTLADAGFM
KQYGECLGDINARDLICAQKFNGLTVLPPLLTDDMIAAYTAALVSGTATAG
WTFGAGAALQIPFAMQMAYRFNGIGVTQNVLYENQKQIANQFNKAISQIQE
SLTTTSTALGKLQDVVNQNAQALNTLVKQLSSNFGAISSVLNDILSRLDKV
EAEVQIDRLITGRLQSLQTYVTQQLIRAAEIRASANLAATKMSECVLGQSK
RVDFCGKGYHLMSFPQAAPHGVVFLHVTYVPSQERNFTTAPAICHEGKAYF
PREGVFVFNGTSWFITQRNFFSPQIITTDNTFVSGNCDVVIGIINNTVYDP
LQPELDSFKEELDKYFKNHTSPDVDLGDISGINASVVNIQKEIDRLNEVAK
NLNESLIDLQELGKYEQYIKWPWYVWLGFIAGLIAIVMVTILLCCMTSCCS
CLKGACSCGSCCKFDEDDSEPVLKGVKLHYT.

In some embodiments, a coronavirus spike protein construct includes a sequence that is at least 90% (e.g., at least 91%, 92%, 93%, 94%, 95%, 96%, 97%, 98%, or 99%) identical to the following sequence:

(SEQ ID NO: 66)
VNLTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTF
EYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNFRVQ
PTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQT
NSPRAAASVASQSIIAYTMSLGAENSVAYSNNSIAIPTNFTISVTTEILPV
SMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADA
GFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNE
VAKNLNESLIDLQELGKYEQYIKWPGGGSGGGSHHHHHHHHHH.

In some embodiments, a coronavirus spike protein construct includes the following sequence: VNLTTRTQLPPAYTNSFTRGVYYPDKVFRSSVLHSTQDLFLPFFSNVTWFHAIHVSGTNGTKRFDNP VLPFNDGVYFASTEKSNIIRGWIFGTTLDSKTQSLLIVNNATNVVIKVCEFQFCNDPFLGVYYHKNNKS WMESEFRVYSSANNCTFEYVSQPFLMDLEGKQGNFKNLREFVFKNIDGYFKIYSKHTPINLVRDLPQ GFSALEPLVDLPIGINITRFQTLLALHRSYLTPGDSSSGWTAGAAAYYVGYLQPRTFLLKYNENGTITD AVDCALDPLSETKCTLKSFTVEKGIYQTSNFRVQPTESIVRFPNITNLCPFGEVFNATRFASVYAWNR KRISNCVADYSVLYNSASFSTFKCYGVSPTKLNDLCFTNVYADSFVIRGDEVRQIAPGQTGKIADYNY KLPDDFTGCVIAWNSNNLDSKVGGNYNYLYRLFRKSNLKPFERDISTEIYQAGSTPCNGVEGFNCYF PLQSYGFQPTNGVGYQPYRVVVLSFELLHAPATVCGPKKSTNLVKNKCVNFNFNGLTGTGVLTESNK KFLPFQQFGRDIADTTDAVRDPQTLEILDITPCSFGGVSVITPGTNTSNQVAVLYQDVNCTEVPVAIHA DQLTPTWRVYSTGSNVFQTRAGCLIGAEHVNNSYECDIPIGAGICASYQTQTNSPRAAASVASQSIIA YTMSLGAENSVAYSNNSIAIPTNFTISVTTEILPVSMTKTSVDCTMYICGDSTECSNLLLQYGSFCTQLN RALTGIAVEQDKNTQEVFAQVKQIYKTPPIKDFGGFNFSQILPDPSKPSKRSFIEDLLFNKVTLADAGFI KQYGDCLGDIAARDLICAQKFNGLTVLPPLLTDEMIAQYTSALLAGTITSGWTFGAGAALQIPFAMQMA YRFNGIGVTQNVLYENQKLIANQFNSAIGKIQDSLSSTASALGKLQDVVNQNAQALNTLVKQLSSNFG AISSVLNDILSRLDKVEAEVQIDRLITGRLQSLQTYVTQQLIRAAEIRASANLAATKMSECVLGQSKRVD FCGKGYHLMSFPQSAPHGWFLHVTYVPAQEKNFTTAPAICHDGKAHFPREGVFVSNGTHWFVTQR NFYEPQIITTDNTFVSGNCDVVIGIVNNTVYDPLQPELDSFKEELDKYFKNHTSPDVDLGDISGINASVV NIQKEIDRLNEVAKNLNESLIDLQELGKYEQYIKWPGGGSGGGSHHHHHHHHHH (SEQ ID NO:66).

This protein construct includes a ten-histidine tag (HHHHHHHHHH; SEQ ID NO:67) linked to the spike protein sequence with a GGGSGGGS (SEQ ID NO:62) linker. The spike protein has the following mutations to stabilize the trimer: R683A, R685A. This construct is available from ACROBiosystems under product number SPN-052H2.

In some embodiments, the peptide of the coronavirus spike protein corresponds to a receptor binding domain of the coronavirus spike protein that specifically binds angiotensin-converting enzyme 2 (ACE2). The region of the SARS-CoV-2 spike protein, which is known to interact with the ACE2 receptor, corresponds to amino acids 323-502 on the 1255 amino acid protein (SEQ ID NO: 14) having the amino acid sequence of sequence of CPFGEVFNATKFPSVYAWERKKISNCVADYSVLYNSTFFSTFKCYGVSATKLNDLCFSNVYADSFVV KGDDVRQIAPGQTGVIADYNYKLPDDFMGCVLAWNTRNIDATSTGNYNYKYRYLRHGKLRPFERDIS NVPFSPDGKPCTPPALNCYWPLNDYGFYTTTGIGYQPYRVWLSFE (SEQ ID NO:17), and thus acts as a region-binding domain (RBD).

In some embodiments, the coronavirus spike protein or peptide of the invention described herein has an amino acid sequence that is identical to a fragment of the SARS-CoV-2 spike protein RBD. In some embodiments, the coronavirus spike protein or peptide of the invention described herein has an amino acid sequence that is at least 90% (e.g., at least 91%, 92%, 93%, 94%, 95%, 96%, 97%, 98%, or 99%) identical to RVQPTESIVRFPNITNLCPFGEVFNATRFASVYAWNRKRISNCVADYSVLYNSASFSTFK CYGVSPTKLNDLCFTNVYADSFVIRGDEVRQIAPGQTGKIADYNYKLPDDFTGCVIAWNSNNLDSKVG GNYNYLYRLFRKSNLKPFERDISTEIYQAGSTPCNGVEGFNCYFPLQSYGFQPTNGVGYQPYRVVVL SFELLHAPATVCGPKKSTNLVKNKCVNFNFNGLTGTGVLTESNKKFLPFQQFGRDIADTTDAVRDPQ TLEILDITPCS (SEQ ID NO: 3). In some embodiments, the coronavirus spike protein peptide has an amino acid sequence of SEQ ID NO: 3.

In some embodiments, the coronavirus spike protein or spike RBD contains one or more mutations. A mutation may be the N501Y mutation detected in a variant in the United Kingdom (202012/01), the A67V, 69del, 70del, 144del, E484K, D614G, Q677H, and F888L mutations detected in a variant in the United Kingdom and Nigeria (the 20A/S:484K variant), the 69del, 70del, 144del, N501Y, A570D, D614G, P681H, T716I, S982A, and D1118H mutations detected in a variant in the United Kingdom (the B.1.1.7 variant, also known as the Alpha variant, or 20I/501Y.V1 variant), the T951, D253G and D614G mutations detected in a variant in the United States, D80G, 144del, F157S, L452R, D614G, and D950H mutations detected in a variant in the United States (the 20C variant), the L452R and D614G mutations found in the United States (the B.1.472 variant also known as the 20C/S:452R variant), the S131, W152C, L452R, D614G mutations detected in a variant in the United States (the B.1.429 variant also known as the 20C/S:452R variant), the L18F, T20N, P26S, D138Y, R190S, K417T, E484K, N501Y, D614G, H655Y, and T1027I mutations detected in a variant in Brazil (the P.1 variant, also known as the Gamma variant, or the 20J/501Y.V3 variant), the E484K, D614G, and V1176F mutations detected in a variant in Brazil (the 20J variant), the L18F, the L452R, E484Q, and D614G mutations found in the variant in India (the 20A variant), the G142D, E154K, L452R, E484Q, D614G, P681R, and Q1071H mutations found in a variant in India (the 20A/S:154K variant), the T19R, G142D, L452R, E484Q, D614G, P681R, and D950N mutations found in a variant in India (the B.1.617.2 variant also known as the Delta variant, the 20A/S:478K variant, or the 20J variant), or the combination of N501&, K417N, and E484K mutations (with or without the D80A, D215G, 241del, 242del, 243del, D614G, and A701V mutations) detected in a variant in South Africa (the B.1.351 variant also known as the Beta variant, 501.V2 variant, or 501.V2, 20H/501Y.V2). The numbering of the variant mutations is relative to the full-length spike protein.

The spike RBD may contain any of the SARS-CoV2 variant mutations. In some embodiments, spike RBD has at least 90% (e.g., 91% 92%, 93%, 94%, 95%, 96%, 97%, 98%, or 99%) sequence identity to the sequence of any of the forementioned SARS-CoV2 variants. In one embodiment, the spike RBD has at least 90% (e.g., 91% 92%, 93%, 94%, 95%, 96%, 97%, 98%, or 99%) sequence identity to the sequence of the B.1.351 variant.

In some embodiments, the spike RBD contains the N501Y mutation and includes the sequence shown below:

(SEQ ID NO: 69)
RVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTE
IYQAGSTPCNGVEGFNCYFPLQSYGFQPTYGVGYQPYRVVVLSFELLHAPA
TVCGPKKSTNLVKNKCVNF.

In some embodiments, a histidine-tag is added to the C-terminus of the sequence of SEQ ID NO:69. In some embodiments, the histidine-tag sequence is: AHHHHHHHHHH (SEQ ID NO:70).

In some embodiments, the coronavirus antigen is a coronavirus nucleocapsid protein or a peptide thereof. In some embodiments the coronavirus nucleocapsid protein includes a sequence that is at least 90% (e.g., at least 91%, 92%, 93%, 94%, 95%, 96%, 97%, 98%, or 99%) identical to the following amino acid sequence:

(SEQ ID NO: 68)
MSDNGPQNQRNAPRITFGGPSDSTGSNQNGERSGARSKQRRPQGLPNNTAS
WFTALTQHGKEDLKFPRGQGVPINTNSSPDDQIGYYRRATRRIRGGDGKMK
DLSPRWYFYYLGTGPEAGLPYGANKDGIIWVATEGALNTPKDHIGTRNPAN
NAAIVLQLPQGTTLPKGFYAEGSRGGSQASSRSSSRSRNSSRNSTPGSSRG
TSPARMAGNGGDAALALLLLDRLNQLESKMSGKGQQQQGQTVTKKSAAEAS
KKPRQKRTATKAYNVTQAFGRRGPEQTQGNFGDQELIRQGTDYKHWPQIAQ
FAPSASAFFGMSRIGMEVTPSGTWLTYTGAIKLDDKDPNFKDQVILLNKHI
DAYKTFPPTEPKKDKKKKADETQALPQRQKKQQTVTLLPAADLDDFSKQLQ
QSMSSADSTQA.

In some embodiments, the coronavirus nucleocapsid protein includes the amino acid sequence of SEQ ID NO:68.

In some embodiments, a coronavirus nucleocapsid protein construct includes the following sequence:

(SEQ ID NO: 63)
MSDNGPQNQRNAPRITFGGPSDSTGSNQNGERSGARSKQRRPQGLPNNTAS
WFTALTQHGKEDLKFPRGQGVPINTNSSPDDQIGYYRRATRRIRGGDGKMK
DLSPRWYFYYLGTGPEAGLPYGANKDGIIWVATEGALNTPKDHIGTRNPAN
NAAIVLQLPQGTTLPKGFYAEGSRGGSQASSRSSSRSRNSSRNSTPGSSRG
TSPARMAGNGGDAALALLLLDRLNQLESKMSGKGQQQQGQTVTKKSAAEAS
KKPRQKRTATKAYNVTQAFGRRGPEQTQGNFGDQELIRQGTDYKHWPQIAQ
FAPSASAFFGMSRIGMEVTPSGTWLTYTGAIKLDDKDPNFKDQVILLNKHI
DAYKTFPPTEPKKDKKKKADETQALPQRQKKQQTVTLLPAADLDDFSKQLQ
QSMSSADSTQAENLYFQGHHHHHH.

This construct includes a cleavage site for a tobacco etch virus (TEV) protease (ENLYFQG; SEQ ID NO:64) between the nucleocapsid protein sequence and the six-histidine tag (HHHHHH; SEQ ID NO:65), and is available from ACROBiosystems under product number NUN-05227.

In some embodiments, the coronavirus spike protein or peptide thereof or the coronavirus nucleocapsid protein or peptide thereof includes one or more tags (e.g., a histidine tag or an Avi tag).

A tag may be used for, for example, protein purification (e.g., affinity tags), to increase the solubility of a protein, to alter chromatographic properties, to give a fluorescent read out, or another purpose. Protein tags include but are not limited to a chitin binding protein (CBP) tag, a maltose binding protein (MBP) tag, a Strep-tag, a glutathione-S-transferase (GST) tag, a histidine tag, an AviTag, a C-tag, a calmodulin tag, an E-tag, a FLAG tag, a human influenza hemagglutinin (HA) tag, a Myc, an S-tag, and an NE-tag. In some embodiments, the coronavirus spike protein or peptide has a histidine tag. In some embodiments, the coronavirus spike protein has an Avi tag. In other embodiments, the coronavirus spike protein or peptide has both a histidine and an Avi tag.

In some embodiments, the coronavirus spike protein or peptide thereof or the coronavirus nucleocapsid protein or peptide thereof includes a protease cleavage site. In some embodiments, the protease cleavage site is between coronavirus spike protein, coronavirus nucleocapsid protein, or peptide sequence and a tag. In some embodiments the protease cleavage site is for TEV. In some embodiments, the TEV cleavage site has the amino acids sequence ENLYFQG (SEQ ID NO:64).

Other examples for predicted immunogenic epitopes that may give rise to an immune response can be found throughout literature (Grifoni et al. Cell Host Microbe. 2020; 27(4): 671-80; Prachar et al. bioRxiv. 2020: 2020.03.20.000794; Chour et al. medRxiv. 2020; 2020.05.04.20085779) and SARS-CoV-2 antigens' vendors' websites (e.g., Sino Biological, Creative Diagnostics, Sengenics, ABclonal Technology). Prediction tools for identifying immunogenic regions based on MHC binding ability are also widely available.

Alternatively, nucleic acids, such as messenger RNA (mRNA), that encode the coronavirus antigen (e.g., a coronavirus spike protein or peptide thereof or a coronavirus nucleocapsid protein or peptide thereof) may be administered. Once injected, the mRNA enters the cell's cytoplasm where it is translated into the desired protein or peptide, that can ultimately activate cellular and humoral immune response. For effective expression of the coronavirus spike protein or peptide, the mRNA will be synthesized to comprise the following: 5′ cap-5′ untranslated region (UTR)—antigen-encoding sequence-3′ untranslated region (UTR)—poly A tail. The design of the 5′ UTR and 3′ UTR are important for mRNA stability, translation, protein production, and structure; there are several online tools that optimize the design of 5′ UTR and 3′ UTR based on mRNA of interest. The coronavirus spike protein or peptide-encoding sequence can be any mRNA sequence that codes for a specific protein or protein subunit; for example, mRNA that encodes SARS-CoV-2 spike protein, spike RBD domain, spike S1 domain, etc. The mRNA may also be non-modified, nucleoside-modified, or self-amplifying. To increase potency, stability, and protein yield, the mRNA may be subject to codon optimization and use of modified nucleosides. For example, incorporation of modified uridines or modified cytidine may be done to avoid premature recognition by innate immune molecules and improve efficiency of translation.

In some embodiments, the mRNA that encodes the coronavirus antigen (e.g., a coronavirus spike protein or peptide thereof or a coronavirus nucleocapsid protein or peptide thereof) is formulated in a lipid nanoparticle (LNP). Lipid nanoparticles typically comprise ionizable cationic lipid, non-cationic lipid, sterol, and PEG lipid components along with the mRNA of interest (e.g., an mRNA encoding a coronavirus antigen such as a coronavirus spike protein or peptide thereof). The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; and PCT/US2016/069491; all of which are incorporated by reference herein in their entirety. The mRNA that encodes the coronavirus antigen may be formulated in a lipid nanoparticle. In some embodiments, the lipid nanoparticle includes at least one ionizable cationic lipid, at least one non-cationic lipid, at least one sterol, and/or at least one polyethylene glycol (PEG)-modified lipid.

The lipid composition of the lipid nanoparticle composition in which the mRNA encoding the coronavirus antigen is formulated can include one or more phospholipids, for example, one or more saturated or (poly)unsaturated phospholipids or a combination thereof. In general, phospholipids include a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

The lipid composition in which the mRNA encoding the coronavirus antigen is formulated can comprise one or more structural lipids. As used herein, the term structural lipid refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol.

The lipid composition in which the mRNA encoding the coronavirus antigen is formulated can include one or more a polyethylene glycol (PEG) lipid. In some embodiments, the PEG-lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

The mRNA that encodes the coronavirus antigen may be encoded within a recombinant vector. The vectors can be used to deliver the mRNA that encodes the coronavirus antigen. The vector may be a mammalian, a viral, or a bacterial expression vector.

The vectors may be, for example, a plasmid, an artificial chromosome (e.g., a BAG, PAC, or YAC), or a virus or phage vector, and may optionally include a promoter, enhancer, or regulator for the expression of the polynucleotide. The vector may also contain one or more selectable marker genes, for example an ampicillin, neomycin, and/or kanamycin resistance gene in the case of a bacterial plasmid or a resistance gene for a fungal vector. Vectors may be used in vitro, for example, for the production of DNA or RNA or used to transfect or transform a host cell, for example, a mammalian host cell, e.g., for the production of protein encoded by the vector. The vectors may also be adapted to be used in vivo, for example in a method of DNA vaccination, RNA vaccination, or gene therapy.

Viral genomes provide a rich source of vectors that can be used for the efficient delivery of the mRNA encoding the coronavirus antigen into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors that can be used to deliver the mRNA encoding the coronavirus antigen include a retrovirus, adenovirus (e.g., Ad2, Ad5, Ad11, Ad12, Ad24, Ad26, Ad34, Ad35, Ad40, Ad48, Ad49, Ad50, Ad52 (e.g., a RhAd52), Ad59 (e.g., a RhAd59), and Pan9 (also known as AdC68)), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding immunogens (e.g., polypeptides) include Norwalk virus, togavirus, coronavirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). These adenovirus vectors can be derived from, for example, human, chimpanzee, or rhesus adenoviruses. Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030); incorporated herein in its entirety by reference. The nucleic acid material (e.g., including a nucleic acid molecule) of the viral vector may be encapsulated, e.g., in a lipid membrane or by structural proteins (e.g., capsid proteins), that may include one or more viral polypeptides (e.g., a glycoprotein). The viral vector can be used to infect cells of a subject, which, in turn, promotes the translation of the heterologous gene(s) of the viral vector into the immunogens.

Adenoviral vectors disclosed in International Patent Application Publications WO 2006/040330 and WO 2007/104792, each incorporated by reference herein, are particularly useful as vectors. These adenoviral vectors can encode and/or deliver one or more of the immunogens (e.g., SARS-CoV-2 polypeptides) to treat a subject having a pathological condition associated with a viral infection (e.g., a SARS-CoV-2 infection). In some embodiments, one or more recombinant adenovirus vectors can be administered to the subject in order to express more than one type of immunogen (e.g., SARS-CoV-2 polypeptide). Besides adenoviral vectors, other viral vectors and techniques are known in the art that can be used to facilitate delivery and/or expression of one or more of the immunogens in a subject (e.g., a human). These viruses include poxviruses (e.g., vaccinia virus and modified vaccinia virus Ankara (MVA); see, e.g., U.S. Pat. Nos. 4,603,112 and 5,762,938, each incorporated by reference herein), herpesviruses, togaviruses (e.g., Venezuelan Equine Encephalitis virus; see, e.g., U.S. Pat. No. 5,643,576, incorporated by reference herein), picornaviruses (e.g., poliovirus; see, e.g., U.S. Pat. No. 5,639,649, incorporated by reference herein), baculoviruses, and others described by Wattanapitayakul and Bauer (Biomed. Pharmacother. 54:487 (2000), incorporated by reference herein).

In some embodiments, the mRNA encoding a coronavirus antigen is incorporated into a recombinant AAV (rAAV) vectors and/or virions in order to facilitate their introduction into a cell. rAAV vectors useful in the compositions and methods described herein are recombinant polynucleotide constructs that include (1) a heterologous sequence to be expressed (e.g., a polynucleotide encoding a coronavirus antigen to be expressed) and (2) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The mRNA encoding a coronavirus antigen can be incorporated into a rAAV virion in order to facilitate introduction of the mRNA encoding a coronavirus antigen into a cell. The capsid proteins of an AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

Useful rAAV virions include those derived from a variety of AAV serotypes including AAV1, AAV2, AAV3, AAV4, AAV5, AAV6, AAV7, AAV8, AAV9, AAV10, AAV11, AAV12 rh10, rh39, rh43, rh74, and Anc80. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

AAV vectors may be pseudotyped vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, AAV9, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting an AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

Gene transfer techniques using these viruses are known to those skilled in the art. Retrovirus vectors for example may be used to stably integrate the polynucleotide into the host genome, although such recombination is not preferred. Replication-defective adenovirus vectors by contrast remain episomal and therefore allow transient expression.

Vectors capable of driving expression in insect cells (for example baculovirus vectors), in human cells, in yeast or in bacteria may be employed in order to produce quantities of coronavirus antigen encoded by the mRNA, for example, for use as subunit vaccines or in immunoassays.

### Adjuvants

In some embodiments, an immunogenic composition described herein may include one or more adjuvants. An adjuvant refers to a substance that cause stimulation of the immune system. In this context, an adjuvant is used to enhance an immune response to one or more antigens (e.g., a coronavirus antigen (e.g., a coronavirus spike protein or a peptide thereof, and/or a coronavirus nucleocapsid protein or peptide thereof, or a nucleic acid sequence encoding the same)). An adjuvant may be administered to a subject before, in combination with, or after administration of the antigens (e.g., a coronavirus antigen (e.g., a coronavirus spike protein or a peptide thereof, and/or a coronavirus nucleocapsid protein or peptide thereof, or a nucleic acid sequence encoding the same)). In some embodiments, an additional adjuvant is administered to the subject in combination with the CpG-amphiphile and the coronavirus antigen (e.g., a coronavirus spike protein or a peptide thereof, and/or a coronavirus nucleocapsid protein or peptide thereof, or a nucleic acid sequence encoding the same) described herein. In some embodiments, an adjuvant may be conjugated to a lipid. The adjuvant may be without limitation lipids (e.g., monophosphoryl lipid A (MPLA)), alum (e.g., aluminum hydroxide, aluminum phosphate); Freund's adjuvant; saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS1312, water-based nanoparticles combined with a soluble immunostimulant, Seppic), and CDNs (cyclic di-nucleotides).

Adjuvants may be toll-like receptor (TLR) ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants 5 acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

### Pharmaceutical Compositions and Preparations

Described herein are pharmaceutical compositions of the invention including a CpG-amphiphile and a coronavirus antigen (e.g., a coronavirus spike protein or a peptide thereof, and/or a coronavirus nucleocapsid protein or peptide thereof, or a nucleic acid sequence encoding the same). In addition to a therapeutic amount of the CpG-amphiphile and a coronavirus antigen (e.g., a coronavirus spike protein or a peptide thereof, and/or a coronavirus nucleocapsid protein or peptide thereof, or a nucleic acid sequence encoding the same), the pharmaceutical compositions may contain a pharmaceutically acceptable carrier or excipient, which can be formulated by methods known to those skilled in the art. In other embodiments, pharmaceutical compositions of the invention may contain nucleic acid molecules encoding one or more coronavirus antigens (e.g., coronavirus spike protein or a peptide thereof, and/or a coronavirus nucleocapsid protein or peptide thereof) described herein (e.g., in a vector, such as a viral vector). The nucleic acid molecule encoding the coronavirus antigen (e.g., a coronavirus spike protein or a peptide thereof, and/or a coronavirus nucleocapsid protein or peptide thereof) thereof described herein may be cloned into an appropriate expression vector, which may be delivered via well-known methods in gene therapy.

Acceptable carriers and excipients in the pharmaceutical compositions of the CpG-amphiphile and the coronavirus antigen (e.g., a coronavirus spike protein or a peptide thereof, and/or a coronavirus nucleocapsid protein or peptide thereof, or a nucleic acid sequence encoding the same) are nontoxic to recipients at the dosages and concentrations employed. In certain embodiments, the formulation material(s) are for subcutaneous (s.c.) and/or intravenous (i.v.) administration. In some embodiments, administration is by inhalation or intranasal administration. In some embodiments, the formulation material(s) are for intratracheal administration. In some embodiments, the formulation material(s) are for administration by inhalation during mechanical ventilation. In some embodiments, the pharmaceutical composition can contain formulation materials for modifying, maintaining, or preserving, for example, the pH, osmolality, viscosity, clarity, color, isotonicity, odor, sterility, stability, rate of dissolution or release, adsorption, or penetration of the composition. In some embodiments, suitable formulation materials include, but are not limited to, amino acids (such as glycine, glutamine, asparagine, arginine or lysine); antimicrobials; antioxidants (such as ascorbic acid, methionine, sodium sulfite or sodium hydrogen-sulfite); buffers (such as borate, bicarbonate, Tris-HCl, citrates, HEPES, TAE, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrosemannose or dextran); proteins (such as human serum albumin, gelatin, dextran, and immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as hexamethonium chloride, octadecyldimethylbenzyl ammonium chloride, resorcinol, and benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the optimal pharmaceutical composition will be determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the amphiphilic conjugate.

In some embodiments, the primary vehicle or carrier in a pharmaceutical composition, including a CpG-amphiphile and a coronavirus antigen (e.g., a coronavirus spike protein or a peptide thereof, and/or a coronavirus nucleocapsid protein or peptide thereof, or a nucleic acid sequence encoding the same), can be either aqueous or non-aqueous in nature. For example, in some embodiments, a suitable vehicle or carrier can be water for injection, physiological saline solution, or artificial cerebrospinal fluid, possibly supplemented with other materials common in compositions for parenteral administration. In some embodiments, the saline includes isotonic phosphate-buffered saline. In certain embodiments, neutral buffered saline or saline mixed with serum albumin are further exemplary vehicles. In some embodiments, pharmaceutical compositions include Tris buffer of about pH 7.0-8.5, or acetate buffer of about pH 4.0-5.5, which can further include sorbitol or a suitable substitute therefor. In some embodiments, a composition including a CpG-amphiphile or a coronavirus antigen (e.g., a coronavirus spike protein or a peptide thereof, and/or a coronavirus nucleocapsid protein or peptide thereof, or a nucleic acid sequence encoding the same) can be prepared for storage by mixing the selected composition having the desired degree of purity with optional formulation agents (Remington's Pharmaceutical Sciences, supra) in the form of a lyophilized cake or an aqueous solution. Further, in some embodiments, a composition including a CpG-amphiphile or a coronavirus antigen (e.g., a coronavirus spike protein or a peptide thereof, and/or a coronavirus nucleocapsid protein or peptide thereof, or a nucleic acid sequence encoding the same), can be formulated as a lyophilizate using appropriate excipients such as sucrose.

In some embodiments, the pharmaceutical composition may be selected for parenteral delivery. The preparation of such pharmaceutically acceptable compositions is within the ability of one skilled in the art.

In some embodiments, the formulation components are present in concentrations that are acceptable to the site of administration. In some embodiments, buffers are used to maintain the composition at physiological pH or at a slightly lower pH, typically within a pH range of from about 5 to about 8.

In some embodiments, when parenteral administration is contemplated, a therapeutic composition can be in the form of a pyrogen-free, parenterally acceptable aqueous solution including a CpG-amphiphile and a coronavirus antigen (e.g., a coronavirus spike protein or a peptide thereof, and/or a coronavirus nucleocapsid protein or peptide thereof, or a nucleic acid sequence encoding the same), in a pharmaceutically acceptable vehicle. In some embodiments, a vehicle for parenteral injection is sterile distilled water in which a CpG-amphiphile or a coronavirus antigen (e.g., a coronavirus spike protein or a peptide thereof, and/or a coronavirus nucleocapsid protein or peptide thereof, or a nucleic acid sequence encoding the same) is formulated as a sterile, isotonic solution, properly preserved. In some embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some embodiments, hyaluronic acid can also be used, and can have the effect of promoting sustained duration in the circulation. In some embodiments, implantable drug delivery devices can be used to introduce the desired molecule.

The pharmaceutical composition may be administered in therapeutically effective amount such as to induce an immune response. The therapeutically effective amount of the CpG-amphiphile and the coronavirus protein or peptide, included in the pharmaceutical preparations may be determined by one of skill in art, such that the dosage (e.g., a dose within the range of 0.01-100 mg/kg of body weight) induces an immune response in the subject.

Vectors may be used as in vivo nucleic acid delivery vehicle include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, a vector can include internal ribosome entry site (IRES) that allows the expression of multiple coronavirus antigens (e.g., a coronavirus spike protein, a peptide thereof, or a nucleic acid sequence encoding the same) described herein. Other vehicles and methods for nucleic acid delivery are described in U.S. Pat. Nos. 5,972,707, 5,697,901, and 6,261,554, each of which is incorporated by reference herein in its entirety. Other methods of producing pharmaceutical compositions are described in, e.g., U.S. Pat. Nos. 5,478,925, 8,603,778, 7,662,367, and 7,892,558, all of which are incorporated by reference herein in their entireties.

### Routes, Dosage, and Timing of Administration

Pharmaceutical compositions of the invention that contain a CpG-amphiphile and a coronavirus antigen (e.g., a coronavirus spike protein or a peptide thereof, and/or a coronavirus nucleocapsid protein or a peptide thereof, or a nucleic acid sequence encoding the same) described herein as the therapeutic agents may be formulated for parenteral administration, subcutaneous administration, intravenous administration, intramuscular administration, intranasal administration, inhalation, intratracheal administration, or administration by inhalation during mechanical ventilation. Methods of administering therapeutic proteins are known in the art. See, for example, U.S. Pat. Nos. 6,174,529, 6,613,332, 8,518,869, 7,402,155, and 6,591,129, and US Patent Application Publication Nos. US20140051634, WO1993000077, and US20110184145, the disclosures of which are incorporated by reference in their entireties.

One or more of these methods may be used to administer a pharmaceutical composition of the invention that contains a CpG-amphiphile and a coronavirus antigen (e.g., a coronavirus spike protein or a peptide thereof, and/or a coronavirus nucleocapsid protein or peptide thereof, or a nucleic acid sequence encoding the same) described herein. For injectable formulations, various effective pharmaceutical carriers are known in the art. See, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986). The dosage of the pharmaceutical compositions of the invention depends on factors including the route of administration and the physical characteristics, e.g., age, weight, general health, of the subject. Typically, the amount of a CpG-amphiphile and a coronavirus antigen (e.g., a coronavirus spike protein or a peptide thereof, and/or a coronavirus nucleocapsid protein or peptide thereof, or a nucleic acid sequence encoding the same) described herein contained within a single dose may be an amount that effectively induces an immune response in the subject without inducing significant toxicity. A pharmaceutical composition of the invention may include a dosage of a CpG-amphiphile and a coronavirus antigen (e.g., a coronavirus spike protein or a peptide thereof, and/or a coronavirus nucleocapsid protein or peptide thereof, or a nucleic acid sequence encoding the same) described herein ranging from 0.001 to 500 mg (e.g., 0.01, 0.05, 0.1, 0.2, 0.3, 0.5, 0.7, 0.8, 1 mg, 2 mg, 3 mg, 4 mg, 5 mg, 10 mg, 15 mg, 20 mg, 30 mg, 50 mg, 100 mg, 250 mg, or 500 mg) and, in a more specific embodiment, about 0.1 to about 100 mg. The dosage may be adapted by the clinician in accordance with the different parameters of the subject.

In particular embodiments, the subject receives a dosage of about 10 μg to about 1.0 mg of the coronavirus antigen (e.g., a coronavirus spike protein or a peptide thereof, and/or a coronavirus nucleocapsid protein or peptide thereof, or a nucleic acid sequence encoding the same). In particular, the dosage of the coronavirus antigen administered is about 40 μg to 60 μg, is about 50 μg to 70 μg, is about 50 μg to 150 μg, is about 70 μg to 150 μg, is about 100 μg to 150 μg, is about 100 μg to 200 μg, is about 140 μg to 250 μg, is about 200 μg to 300 μg, is about 250 μg to 500 μg, is about 300 μg to 600 μg, or is about 500 μg to 1.0 mg. In particular, the dosage of the coronavirus antigen administered to the subject may be about 10 μg, 20 μg, 30 μg, 40 μg, 50 μg, 60, pg, 70 μg, 80 μg, 90 μg, 100 μg, 110 μg, 120 μg, 130 μg, 140 μg, 150 μg, 200 μg, 250 μg, 300 μg, 400 μg, 500 μg, 600 μg, 700 μg, 800 μg, 900 μg, or 1 mg. The subject also may receive a dosage in a range between any two of these particular dosages of the coronavirus antigen.

In particular embodiments, the dosage of the CpG amphiphile is about 0.1 mg to 20 mg. In particular, the dosage of the CpG amphiphile administered is about 0.1 mg to 1.0 mg, is about 0.5 mg to 3.0 mg, is about 1.0 mg to 5.0 mg, is about 2.0 to 5.0 mg, is about 3.0 to 5.0 mg, is about 3.0 mg to 10.0 mg, is about 4.0 mg to 12.0 mg, is about 5.0 mg to 15.0 mg or is about 5.0 mg to 20 mg. The particular dosage of the CpG amphiphile administered to the subject may be about 0.1 mg, 0.2 mg, 0.3 mg, 0.4 mg, 0.5 mg, 1.0 mg, 2.0 mg, 3.0 mg, 4.0 mg, 5.0 mg, 6.0 mg, 7.0 mg, 8.0 mg, 9.0 mg, 10.0 mg, 11.0 mg, 12.0 mg, 13.0 mg, 14.0 mg, 15.0 mg, 16.0 mg, 17.0 mg, 18.0 mg, 19.0 mg, or 20.0 mg. The subject also may receive a dosage in a range between any two of these particular dosages of the CpG amphiphile.

Pharmaceutical compositions of the invention that contain a CpG-amphiphile and a coronavirus antigen (e.g., a coronavirus spike protein or a peptide thereof, and/or a coronavirus nucleocapsid protein or peptide thereof, or a nucleic acid sequence encoding the same) described herein may be administered to a subject in need thereof, for example, one or more times (e.g., 1-10 times or more) daily, weekly, monthly, biannually, annually, or as medically necessary.

In some embodiments, a trimer of the coronavirus spike protein or peptide is administered to the subject. In some embodiments, an mRNA encoding a coronavirus antigen (e.g., a coronavirus spike protein or a peptide thereof, and/or a coronavirus nucleocapsid protein or peptide thereof, or a nucleic acid sequence encoding the same) is administered to the subject. In some embodiments, the coronavirus antigen (e.g., a coronavirus spike protein or a peptide thereof, and/or a coronavirus nucleocapsid protein or peptide thereof, or a nucleic acid sequence encoding the same) and the CpG-amphiphile are administered concurrently or essentially at the same time to the subject. The CpG-amphiphile and the coronavirus antigen (e.g., a coronavirus spike protein or a peptide thereof, and/or a coronavirus nucleocapsid protein or peptide thereof, or a nucleic acid sequence encoding the same) may be co-formulated, or they may be administered as two separate formulations. In some embodiments, the CpG-amphiphile and the coronavirus antigen (e.g., a coronavirus spike protein or a peptide thereof, and/or a coronavirus nucleocapsid protein or peptide thereof, or a nucleic acid sequence encoding the same) administered sequentially. For example, the CpG-amphiphile may be administered first and the coronavirus antigen (e.g., a coronavirus spike protein or a peptide thereof, and/or a coronavirus nucleocapsid protein or peptide thereof, or a nucleic acid sequence encoding the same) may be administered second, or, in some embodiments, the coronavirus antigen (e.g., a coronavirus spike protein or a peptide thereof, and/or a coronavirus nucleocapsid protein or peptide thereof, or a nucleic acid sequence encoding the same) may be administered first and the CpG-amphiphile is administered second. In some embodiments, the CpG-amphiphile and the coronavirus antigen (e.g., a coronavirus spike protein or a peptide thereof, and/or a coronavirus nucleocapsid protein or peptide thereof, or a nucleic acid sequence encoding the same) are administered with a second adjuvant.

### Methods of Inducing an Immune Response

The invention provides methods of inducing an immune response in a subject by administering a CpG-amphiphile and a coronavirus antigen (e.g., a coronavirus spike protein or a peptide thereof, and/or a coronavirus nucleocapsid protein or peptide thereof, or a nucleic acid sequence encoding the same) to a subject. The subject may be a mammal (e.g., a human, a dog, or a cat). In some embodiments, the subject is a human subject. The immune response is induced in the subject by administering to the subject a therapeutically effective amount of an immunogenic composition or pharmaceutical composition described herein. The immunogenic composition or pharmaceutical composition includes a CpG-amphiphile and a coronavirus antigen (e.g., a coronavirus spike protein or a peptide thereof, and/or a coronavirus nucleocapsid protein or peptide thereof, or a nucleic acid sequence encoding the same) described herein. In some embodiments, the CpG-amphiphile and the coronavirus antigen (e.g., a coronavirus spike protein or a peptide thereof, and/or a coronavirus nucleocapsid protein or peptide thereof, or a nucleic acid sequence encoding the same) may be administered with one or more additional adjuvants. In some embodiments, the CpG-amphiphile and the coronavirus antigen (e.g., a coronavirus spike protein or a peptide thereof, and/or a coronavirus nucleocapsid protein or peptide thereof, or a nucleic acid sequence encoding the same) may be administered without one or more additional adjuvants. In some embodiments, the method includes administering to the subject 1) a therapeutically effective amount of a CpG-amphiphile described herein, and 2) a coronavirus antigen (e.g., a coronavirus spike protein or a peptide thereof, and/or a coronavirus nucleocapsid protein or peptide thereof, or a nucleic acid sequence encoding the same). In some embodiments, the CpG-amphiphile and the coronavirus antigen (e.g., a coronavirus spike protein or a peptide thereof, and/or a coronavirus nucleocapsid protein or peptide thereof, or a nucleic acid sequence encoding the same) are administered substantially simultaneously. In some embodiments, the CpG-amphiphile and the coronavirus antigen (e.g., a coronavirus spike protein or a peptide thereof, and/or a coronavirus nucleocapsid protein or peptide thereof, or a nucleic acid sequence encoding the same) are administered separately. In some embodiments, the CpG-amphiphile is administered first, followed by administering of the coronavirus spike protein or peptide. In some embodiments, the coronavirus antigen (e.g., a coronavirus spike protein or a peptide thereof, and/or a coronavirus nucleocapsid protein or peptide thereof, or a nucleic acid sequence encoding the same) is administered first, followed by administering of the CpG-amphiphile.

In some embodiments, the immune response is protective against SARS-CoV-2 infection.

In some embodiments, the immune response is protective against Covid-19 disease.

In some embodiments, the immune response is protective against severe Covid-19 disease with requirement of assisted ventilation and oxygenation. These patients suffer from acute respiratory distress syndrome. Some patients develop severe cardiovascular damage. Other complications include, e.g., acute cardiac injury, acute kidney injury, septic shock, multi-organ failure, and increased risk of death.

In some embodiments, the immune response is protective against the development of one or more COVID-19 disease symptoms selected from the group consisting of fever, sore throat, runny nose, sneezing, nasal congestion, snoring, coughing, dry cough, shortness of breath, difficulty breathing, persistent pain or pressure in the chest, dyspnea, pneumonia, acute respiratory syndrome, cyanosis, myalgia, headache, encephalopathy, myocardial injury, heart failure, arrhythmia, coagulation dysfunction, acute kidney injury, confusion or inability to arouse, fatigue, and gastrointestinal symptoms.

In some embodiments, the immune response reduces the incidence of one or more COVID-19 disease symptoms selected from the group consisting of fever, sore throat, runny nose, sneezing, nasal congestion, snoring, coughing, dry cough, shortness of breath, difficulty breathing, persistent pain or pressure in the chest, dyspnea, pneumonia, acute respiratory syndrome, cyanosis, myalgia, headache, encephalopathy, myocardial injury, heart failure, arrhythmia, coagulation dysfunction, acute kidney injury, confusion or inability to arouse, fatigue, and gastrointestinal symptoms.

In some embodiments, the immune response is therapeutic against one or more COVID-19 disease symptoms selected from the group consisting of fever, sore throat, runny nose, sneezing, nasal congestion, snoring, coughing, dry cough, shortness of breath, difficulty breathing, persistent pain or pressure in the chest, dyspnea, pneumonia, acute respiratory syndrome, cyanosis, myalgia, headache, encephalopathy, myocardial injury, heart failure, arrhythmia, coagulation dysfunction, acute kidney injury, confusion or inability to arouse, fatigue, and gastrointestinal symptoms. The immune response is therapeutic if it reverses, alleviates, ameliorates, inhibits, slows down, or stops the progression or severity of a COVID-19 disease symptom.

In some embodiments, the immune response reduces the likelihood of COVID-19 recurrence or SARS-CoV-2 reinfection.

In some embodiments, the immune response reduces the likelihood of transmission of SARS-CoV-2.

In some embodiments, the subject is an asymptomatic carrier of SARS-CoV-2.

In some embodiments, the subject has one or more symptoms of COVID-19 selected from the group consisting of fever, sore throat, runny nose, sneezing, nasal congestion, snoring, coughing, dry cough, shortness of breath, difficulty breathing, persistent pain or pressure in the chest, dyspnea, pneumonia, acute respiratory syndrome, cyanosis, myalgia, headache, encephalopathy, myocardial injury, heart failure, arrhythmia, coagulation dysfunction, acute kidney injury, confusion or inability to arouse, fatigue, and gastrointestinal symptoms.

In some embodiments, the subject has been diagnosed with SARS-CoV-2 infection.

In some embodiments, the subject is at high risk of SARS-CoV-2 infection (e.g., medical personnel and/or first responders).

In some embodiments, an immunogenic composition or pharmaceutical composition described herein is administered to a subject who has been in contact with someone who has been diagnosed with a coronavirus infection (e.g., COVID-19) or who has recently travelled or is planning to travel to an area experiencing an outbreak of COVID-19 or other coronavirus infection.

In some embodiments, the spike protein or peptide thereof is comprised within a preparation of an inactivated or killed virus vaccine.

In some embodiments, the spike protein or fragment thereof is in subunit form.

In some embodiments, the nucleic acid encoding the coronavirus spike protein encodes a prefusion stabilized form of the spike protein.

In some embodiments, the nucleic acid encoding the coronavirus spike protein or peptide thereof is comprised within an adenovirus vector.

### Combination Therapies

The invention described herein also provides methods of inducing an immune response in a subject by administering a CpG-amphiphile and a coronavirus antigen in combination with one or more additional therapeutics. The particular combination of therapeutics that can be employed in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of one or more adverse effects). The CpG-amphiphile and coronavirus antigen may be administered in combination with an antiviral agent (e.g., remdesivir), an antiviral vaccine (e.g., a coronavirus vaccine such as a vaccine against SARS-CoV-2), an antibiotic agent, an antifungal agent, an anti-inflammatory agent, an antiparasitic agent, and an immunotherapy agent.

In some embodiments, the antiviral agent may be remdesivir, chloroquine, hydroxychloroquine, baricitinib, lopinavir/ritonavir, interferon beta, umifenovir, favipiravir, tocilizumab, ribavirin or other drugs. In some embodiments, the antiviral agent is remdesivir.

In some embodiments, the antiviral vaccine includes any composition that elicits an immune response in a subject directed against a coronavirus, such as a HCoV-NL3 vaccine, a SARS-CoV-1 vaccine, a SARS-CoV-2 vaccine, or a MERS vaccine. In some embodiments, the antiviral vaccine includes an inactivated or killed virus, such as an HCoV-NL3 virus, a SARS-CoV-1 virus, a SARS-CoV-2 virus, or a MERS virus. In some embodiments, the antiviral vaccine is administered as a heterologous prime or boost or in combination with the immunogenic composition including a CpG-amphiphile described herein.

In some embodiments, the antibiotic agent may be elected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, temocillin, ticarcillin, amoxicillin clavulanate, ampicillin/sulbactam, piperacillin/tazobactam, ticarcillin/clavulanate, bacitracin, colistin, polymyxin b, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, demeclocycline, doxycycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim.

In some embodiments, the antifungal agent may be selected from amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, triazoles, albaconazole, efinaconazole, epoxiconazole, fluconazole, isavuconazole, itraconazole, posaconazole, propiconazole, ravuconazole, terconazole, voriconazole, thiazoles, abafungin, amorolfin, butenafine, naftifine, terbinafine, anidulafungin, caspofungin, micafungin, ciclopirox, flucytosine, griseofulvin, tolnaftate, and undecylenic acid. In some embodiments, the antiparasitic agent may be chloroquine or hydroxychloroquine.

In some embodiments, the anti-inflammatory agent may be dexamethasone. In some embodiments the anti-inflammatory agent may be selected from celecoxib, diclofenac, difunisal, etodolac, ibuprofen, indomethacin, ketoprofen, ketorolac, nabumetone, naproxen, oxaprozin, prednisone, prednisolone, methylprednisolone, metformin, and dexamethasone.

In some embodiments, the immunotherapy agent may be selected from Targretin, Interferon-alpha, Interferon-beta, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat. In some embodiments, the immunotherapy agent is interferon-beta, tocilizumab, or baricitinib. In some embodiments, the immunotherapy agent may include an antibody. In some embodiments, the immunotherapy agent may be convalescent plasma (e.g., human convalescent plasma).

The CpG-amphiphile and the coronavirus antigen and the one or more additional therapeutics may be administered sequentially (e.g., 1 day apart, 2 days apart, 3 days apart, 1 week apart, 1 month apart, 6 months apart, or more) or substantially simultaneously (e.g., within 1 day). The CpG-amphiphile and the coronavirus antigen and the one or more additional therapeutics may be formulated in a single pharmaceutical composition or may be administered as separate pharmaceutical compositions. The CpG-amphiphile and the coronavirus antigen and the one or more additional therapeutics may be administered by the same route of administration or different routes of administration. The two or more agents may be administered at the same frequency or different frequencies.

The additional therapeutic agent may be administered orally, topically, intravenously, intramuscularly, transdermally, intradermally, intra-arterially, intracranially, subcutaneously, intraorbitally, intraventricularly, intraspinally, intraperitoneally, intranasally, intratracheally, or by inhalation during mechanical ventilation. In particular embodiments, the additional therapeutic is administered intravenously or the additional therapeutic agent may be administered in its regulatory approved form. The additional therapeutic agent, or a pharmaceutically acceptable salt thereof, can be administered in a pharmaceutical composition that includes one or more pharmaceutically acceptable carriers, excipients, or diluents. Examples of suitable carriers, excipients, or diluents include, e.g., saline, sterile water, polyalkylene glycols, oils of vegetable origin, hydrogenated napthalenes, suitable buffer, 1,3-butanediol, Ringer's solution and/or sodium chloride solution. Exemplary formulations for parenteral administration can include solutions prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Other exemplary carriers, excipients, or diluents are described in the Handbook of Pharmaceutical Excipients, 6th Edition, Rowe et al., Eds., Pharmaceutical Press (2009), hereby incorporated by reference in its entirety. The additional therapeutic agent may be administered in a pharmaceutical composition useful in the methods of the invention and can take the form of tablets, gelcaps, capsules, pills, powders, granulates, suspensions, emulsions, a sterile solution or suspension, and/or a sustained-release formulation.

### Kits

A kit can include a CpG-amphiphile and a coronavirus antigen (e.g., a coronavirus spike protein or a peptide thereof, and/or a coronavirus nucleocapsid protein or a peptide thereof, or a nucleic acid sequence encoding the same), as disclosed herein, and instructions for use. The kits may include, in one or more suitable containers, a CpG-amphiphile and coronavirus antigen (e.g., a coronavirus spike protein or a peptide thereof, and/or a coronavirus nucleocapsid protein or a peptide thereof, or a nucleic acid sequence encoding the same), one or more controls, and various buffers, reagents, enzymes and other standard ingredients well known in the art. In some embodiments, the kits further include an adjuvant.

The container can include one or more vials, wells, test tubes, flasks, bottles, syringes, or other container means, into which the CpG-amphiphile or the coronavirus antigen (e.g., a coronavirus spike protein or a peptide thereof, and/or a coronavirus nucleocapsid protein or a peptide thereof, or a nucleic acid sequence encoding the same) may be placed, and in some instances, suitably aliquoted. When an additional component is provided, the kit can contain additional containers into which this compound may be placed. The kits can also include a means for containing the CpG-amphiphile and the coronavirus antigen (e.g., a coronavirus spike protein or a peptide thereof, and/or a coronavirus nucleocapsid protein or a peptide thereof, or a nucleic acid sequence encoding the same), and any other reagent containers in close confinement for commercial sale. Such containers may include injection or blow-molded plastic containers into which the desired vials are retained. Containers and/or kits can include labeling with instructions for use and/or warnings.

In some embodiments, the disclosure provides a kit including one or more containers including a composition including a CpG-amphiphile and a composition including a coronavirus antigen (e.g., a coronavirus spike protein or a peptide thereof, and/or a coronavirus nucleocapsid protein or a peptide thereof, or a nucleic acid sequence encoding the same), an optional pharmaceutically acceptable carrier, and a package insert including instructions for administration of the composition for inducing an immune response. In some embodiments, the kit further includes an additional adjuvant and instructions for administration of the adjuvant.

In some embodiments, the disclosure provides a kit including a medicament including a composition including a CpG-amphiphile and a coronavirus antigen (e.g., a coronavirus spike protein or a peptide thereof, and/or a coronavirus nucleocapsid protein or a peptide thereof, or a nucleic acid sequence encoding the same), an optional pharmaceutically acceptable carrier, and a package insert including instructions for administration of the medicament alone or in combination with a composition including an additional adjuvant and an optional pharmaceutically acceptable carrier, for inducing an immune response.

In some embodiments, the disclosure provides a kit including a container including a composition including a CpG-amphiphile and a composition including a coronavirus antigen (e.g., a coronavirus spike protein or a peptide thereof, and/or a coronavirus nucleocapsid protein or a peptide thereof, or a nucleic acid sequence encoding the same), an optional pharmaceutically acceptable carrier, and a package insert including instructions for administration of a composition vaccine for inducing an immune response in a subject. In some embodiments, the kit further includes an additional adjuvant and instructions for administration of the adjuvant for inducing an immune response in a subject.

In addition to the compositions described herein, the kit can include other components or ingredients, such as a container(s) of a solvent or buffer, a stabilizer, a preservative, a flavoring agent (e.g., a bitter antagonist or a sweetener), a fragrance, a dye or coloring agent, for example, to tint or color one or more components in the kit. The kit can also include a second agent for treating a condition or disorder described herein (e.g., a coronavirus infection). Alternatively, other component(s) can be included in the kit, but in different compositions or containers distinct from the composition the CpG-amphiphile and the coronavirus antigen (e.g., a coronavirus spike protein or a peptide thereof, and/or a coronavirus nucleocapsid protein or a peptide thereof, or a nucleic acid sequence encoding the same). In such embodiments, the kit can include instructions for admixing a compound described herein and the other component(s), or for using a compound described herein (e.g., the CpG-amphiphile and the coronavirus antigen (e.g., a coronavirus spike protein or a peptide thereof, and/or a coronavirus nucleocapsid protein or a peptide thereof, or a nucleic acid sequence encoding the same)) together with the other component(s).

A composition described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The containers of the kits can be airtight, waterproof (e.g., impermeable to changes in moisture or evaporation), and/or light-tight.

The kit optionally includes a device suitable for delivery of the composition, e.g., a syringe.

### NUMBERED EMBODIMENTS

Some embodiments of the technology described herein can be defined according to any of the following numbered embodiments. Also encompassed are compositions and kits that include the components used in the methods described herein.
1. A method of inducing an immune response against a coronavirus antigen in a subject, the method comprising administering (1) a CpG-amphiphile and (2) a coronavirus antigen or a nucleic acid sequence encoding the coronavirus antigen to the subject.
2. A CpG-amphiphile and a coronavirus antigen or a nucleic acid sequence encoding the coronavirus antigen for use in inducing an immune response against a coronavirus antigen in a subject, wherein the CpG-amphiphile and the coronavirus antigen or a nucleic acid sequence encoding the coronavirus antigen are formulated for administration to the subject.
3. The method of embodiment 1, wherein the coronavirus antigen is a coronavirus spike protein or a peptide thereof or a nucleic acid sequence encoding the coronavirus spike protein or peptide.
4. The method of embodiment 1 or 3, wherein the CpG-amphiphile comprises a CpG sequence bonded to a lipid.
5. The method of embodiment 1 or 3, the CpG-amphiphile comprises a CpG sequence linked to a lipid by a linker.
6. The method of embodiment 5, wherein the linker comprises a polymer, a string of amino acids, a string of nucleic acids, a polysaccharide, or a combination thereof.
7. The method of embodiment 6, wherein the linker comprises a string of nucleic acids.
8. The method of embodiment 7, wherein the string of nucleic acids comprises between 1 and 50 nucleic acid residues.
9. The method of embodiment 8, wherein the string of nucleic acids comprises between 5 and 30 nucleic acid residues.
10. The method of any one of embodiments 5-9, wherein the string of nucleic acids comprises “N” guanines, wherein N is 1-10.
11. The method of embodiment 6, wherein the linker comprises consecutive polyethylene glycol units.
12. The method of embodiment 11, wherein the linker comprises “N” consecutive polyethylene glycol units, wherein N is between 20 and 80.
13. The method of embodiment 12, wherein the linker comprises “N” consecutive polyethylene glycol units, wherein N is between 30 and 70.
14. The method of embodiment 13, wherein the linker comprises “N” consecutive polyethylene glycol units, wherein N is between 40 and 60.
15. The method of embodiment 14, wherein the linker comprises “N” consecutive polyethylene glycol units, wherein N is between 45 and 55.
16. The method of embodiment 15, wherein the linker comprises 48 consecutive polyethylene glycol units.
17. The method of any one of embodiments 1 and 3-16, wherein the lipid is a diacyl lipid.
18. The method of embodiment 17, wherein the diacyl lipid has the following structure:

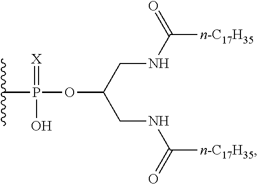

or a salt thereof,
wherein X is O or S.
19. The method of any one of embodiments 1 and 3-18 wherein the CpG sequence comprises the nucleotide sequence 5′-TCGTCGTTTTGTCGTTTTGTCGTT-3′ (SEQ ID NO:1).
20. The method of any one of embodiments 1 and 3-18, wherein the CpG sequence comprises the nucleotide sequence of 5′-TCCATGACGTTCCTGACGTT-3′ (SEQ ID NO: 2).
21. The method embodiment 19 or embodiment 20, wherein all internucleoside groups connecting the nucleosides in the CpG sequence are phosphorothioates.
22. The method of any one of embodiments 1 and 3-21, wherein the coronavirus spike protein or peptide thereof is a SARS-CoV-2 spike protein or peptide thereof.
23. The method of any one of embodiments 1 and 3-22, wherein the peptide of the coronavirus spike protein is a receptor binding domain that specifically binds angiotensin-converting enzyme 2 (ACE2).
24. The method of any one of embodiments 1 and 3-23, wherein the peptide of the coronavirus spike protein comprises a polypeptide sequence having at least 90% sequence identity to:

(SEQ ID NO: 3)
RVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPA
TVCGPKKSTNLVKNKCVNFNFNGLTGTGVLTESNKKFLPFQQFGRDIADTT
DAVRDPQTLEILDITPCS.

25. The method of embodiment 24, wherein the peptide of the coronavirus spike protein comprises the polypeptide sequence of:

(SEQ ID NO: 3)
RVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPA
TVCGPKKSTNLVKNKCVNFNFNGLTGTGVLTESNKKFLPFQQFGRDIADTT
DAVRDPQTLEILDITPCS.

26. The method of any one of embodiments 1 and 4-22, wherein the coronavirus antigen is a coronavirus nucleocapsid protein or a peptide thereof.
27. The method of embodiment 26, wherein the coronavirus nucleocapsid protein antigen comprises a polypeptide sequence having at least 90% sequence identity to:

(SEQ ID NO: 68)
MSDNGPQNQRNAPRITFGGPSDSTGSNQNGERSGARSKQRRPQGLPNNTAS
WFTALTQHGKEDLKFPRGQGVPINTNSSPDDQIGYYRRATRRIRGGDGKMK
DLSPRWYFYYLGTGPEAGLPYGANKDGIIWVATEGALNTPKDHIGTRNPAN
NAAIVLQLPQGTTLPKGFYAEGSRGGSQASSRSSSRSRNSSRNSTPGSSRG
TSPARMAGNGGDAALALLLLDRLNQLESKMSGKGQQQQGQTVTKKSAAEAS
KKPRQKRTATKAYNVTQAFGRRGPEQTQGNFGDQELIRQGTDYKHWPQIAQ
FAPSASAFFGMSRIGMEVTPSGTWLTYTGAIKLDDKDPNFKDQVILLNKHI
DAYKTFPPTEPKKDKKKKADETQALPQRQKKQQTVTLLPAADLDDFSKQLQ
QSMSSADSTQA.

28. The method of embodiment 26, wherein the coronavirus nucleocapsid protein antigen comprises the polypeptide sequence of:

(SEQ ID NO: 63)
MSDNGPQNQRNAPRITFGGPSDSTGSNQNGERSGARSKQRRPQGLPNNTAS
WFTALTQHGKEDLKFPRGQGVPINTNSSPDDQIGYYRRATRRIRGGDGKMK
DLSPRWYFYYLGTGPEAGLPYGANKDGIIWVATEGALNTPKDHIGTRNPAN
NAAIVLQLPQGTTLPKGFYAEGSRGGSQASSRSSSRSRNSSRNSTPGSSRG
TSPARMAGNGGDAALALLLLDRLNQLESKMSGKGQQQQGQTVTKKSAAEAS
KKPRQKRTATKAYNVTQAFGRRGPEQTQGNFGDQELIRQGTDYKHWPQIAQ
FAPSASAFFGMSRIGMEVTPSGTWLTYTGAIKLDDKDPNFKDQVILLNKHI
DAYKTFPPTEPKKDKKKKADETQALPQRQKKQQTVTLLPAADLDDFSKQLQ
QSMSSADSTQAENLYFQGHHHHHH.

29. The method of any one of embodiments 1 and 3-28, wherein the coronavirus antigen comprises one or more tags.
30. The method of embodiment 29, wherein the tag is an Avi tag.
31. The method of embodiment 29, wherein the tag is a histidine tag.
32. The method of any one of embodiments 29-31, wherein the coronavirus antigen comprises an Avi tag and a histidine tag.
33. The method of any one of embodiments 29-32, wherein the coronavirus antigen comprises a linker between the polypeptide sequence and the one or more tags.
34. The method of any one of embodiments 1, 3-25, and 29-32, wherein the coronavirus spike protein is administered.
35. The method of embodiment 34, wherein a trimer of the coronavirus spike protein is administered.
36. The method of embodiment 35, wherein the trimer is a trimer of a protein construct comprising the sequence:

(SEQ ID NO: 66)
VNLTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTF
EYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNFRVQ
PTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQT
NSPRAAASVASQSIIAYTMSLGAENSVAYSNNSIAIPTNFTISVTTEILPV
SMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADA
GFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNE
VAKNLNESLIDLQELGKYEQYIKWPGGGSGGGSHHHHHHHHHH.

37. The method of any one of embodiments 1 and 3-36, wherein a coronavirus spike protein, or a peptide thereof, and a coronavirus nucleocapsid protein, or a peptide thereof, are administered.
38. The method of embodiment 37, wherein a trimer of a coronavirus spike protein construct comprising the sequence:

(SEQ ID NO: 66)
VNLTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTF
EYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNFRVQ
PTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQT
NSPRAAASVASQSIIAYTMSLGAENSVAYSNNSIAIPTNFTISVTTEILPV
SMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADA
GFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNE
VAKNLNESLIDLQELGKYEQYIKWPGGGSGGGSHHHHHHHHHH,

and a coronavirus nucleocapsid protein construct having the polypeptide sequence of: MSDNGPQNQRNAPRITFGGPSDSTGSNQNGERSGARSKQRRPQGLPNNTASWFTALTQHGKEDL KFPRGQGVPINTNSSPDDQIGYYRRATRRIRGGDGKMKDLSPRWYFYYLGTGPEAGLPYGANKDGII WVATEGALNTPKDHIGTRNPANNAAIVLQLPQGTTLPKGFYAEGSRGGSQASSRSSSRSRNSSRNS TPGSSRGTSPARMAGNGGDAALALLLLDRLNQLESKMSGKGQQQQGQTVTKKSAAEASKKPRQKR TATKAYNVTQAFGRRGPEQTQGNFGDQELIRQGTDYKHWPQIAQFAPSASAFFGMSRIGMEVTPSG TWLTYTGAIKLDDKDPNFKDQVILLNKHIDAYKTFPPTEPKKDKKKKADETQALPQRQKKQQTVTLLP AADLDDFSKQLQQSMSSADSTQAENLYFQGHHHHHH (SEQ ID NO:63) are administered.
39. The method of any one of embodiments 1, 3-26, 29-32, and 37, wherein an mRNA encoding the coronavirus antigen is administered.
40. The method of any one of embodiments 1 and 3-39, wherein the CpG-amphiphile and the coronavirus antigen or nucleic acid sequence encoding the same are administered concurrently.
41. The method of any one of embodiments 1 and 3-39, wherein the CpG-amphiphile and the coronavirus antigen, or nucleic acid sequence enclosing the same are administered sequentially.
42. The method of embodiment 41, wherein the CpG-amphiphile is administered first, followed by administering of the coronavirus antigen or nucleic acid sequence encoding the same.
43. The method of embodiment 41, wherein said the coronavirus antigen or nucleic acid sequence encoding the same is administered first, followed by administering of CpG-amphiphile.
44. The method of any one of embodiments 1 and 3-43, wherein the method comprises administering a second adjuvant to the subject.
45. The method of any one of embodiments 1 and 3-44, wherein the method comprises administering a coronavirus vaccine to the subject as a prime or a boost.
46. The method of any one of embodiments 1 and 3-45, wherein the CpG-amphiphile is administered subcutaneously, intranasally, intratracheally, or by inhalation during mechanical ventilation.
47. The method of embodiment 46, wherein the CpG-amphiphile is administered subcutaneously.
48. The method of any one of embodiments 1 and 3-47, wherein the coronavirus antigen is administered subcutaneously, intranasally, intratracheally, or by inhalation during mechanical ventilation.
49. The method of any one of embodiments 1 and 3-48, wherein the subject is a mammal.
50. The method of embodiment 49, wherein the subject is a human.
51. A pharmaceutical composition comprising a CpG-amphiphile and a coronavirus antigen, or a nucleic acid sequence encoding the coronavirus antigen, and a pharmaceutically acceptable carrier.
52. The pharmaceutical composition of embodiment 51, wherein the coronavirus antigen is a coronavirus spike protein or a peptide thereof.
53. The pharmaceutical composition of embodiment 51, wherein the coronavirus antigen is a coronavirus nucleocapsid protein or a peptide thereof.
54. The pharmaceutical composition of embodiment 51, wherein the coronavirus antigen comprises a coronavirus spike protein or a peptide thereof and a coronavirus nucleocapsid protein or a peptide thereof.
55. A kit comprising a CpG-amphiphile and a coronavirus antigen or a nucleic acid sequence encoding the coronavirus antigen.
56. The kit of embodiment 55, wherein the coronavirus antigen is a coronavirus spike protein or a peptide thereof.
57. The kit of embodiment 55, wherein the coronavirus antigen is a coronavirus nucleocapsid protein or a peptide thereof.
58. The kit of embodiment 55, wherein the coronavirus antigen comprises a coronavirus spike protein or a peptide thereof and a coronavirus nucleocapsid protein or a peptide thereof.
59. The CpG-amphiphile and a coronavirus antigen, or a nucleic acid sequence encoding the coronavirus antigen for use according to embodiment 2, wherein the coronavirus antigen is a coronavirus spike protein or a peptide thereof or a nucleic acid sequence encoding the coronavirus spike protein or peptide.
60. The CpG-amphiphile and a coronavirus antigen, or a nucleic acid sequence encoding the coronavirus antigen for use according to embodiment 2 or 59, wherein the CpG-amphiphile comprises a CpG sequence bonded to a lipid.
61. The CpG-amphiphile and a coronavirus antigen, or a nucleic acid sequence encoding the coronavirus antigen for use according to embodiment 2 or 59, the CpG-amphiphile comprises a CpG sequence linked to a lipid by a linker.
62. The CpG-amphiphile and a coronavirus antigen, or a nucleic acid sequence encoding the coronavirus antigen for use according to embodiment 61, wherein the linker comprises a polymer, a string of amino acids, a string of nucleic acids, a polysaccharide, or a combination thereof.
63. The CpG-amphiphile and a coronavirus antigen, or a nucleic acid sequence encoding the coronavirus antigen for use according to embodiment 62, wherein the linker comprises a string of nucleic acids.
64. The CpG-amphiphile and a coronavirus antigen, or a nucleic acid sequence encoding the coronavirus antigen for use according to embodiment 63, wherein the string of nucleic acids comprises between 1 and 50 nucleic acid residues.
65. The CpG-amphiphile and a coronavirus antigen, or a nucleic acid sequence encoding the coronavirus antigen for use according to embodiment 64, wherein the string of nucleic acids comprises between 5 and 30 nucleic acid residues.
66. The CpG-amphiphile and a coronavirus antigen, or a nucleic acid sequence encoding the coronavirus antigen for use according to any one of embodiments 61-65, wherein the string of nucleic acids comprises “N” guanines, wherein N is 1-10.
67. The CpG-amphiphile and a coronavirus antigen, or a nucleic acid sequence encoding the coronavirus antigen for use according to embodiment 63, wherein the linker comprises consecutive polyethylene glycol units.
68. The CpG-amphiphile and a coronavirus antigen, or a nucleic acid sequence encoding the coronavirus antigen for use according to embodiment 67, wherein the linker comprises “N” consecutive polyethylene glycol units, wherein N is between 20 and 80.
69. The CpG-amphiphile and a coronavirus antigen, or a nucleic acid sequence encoding the coronavirus antigen for use according to embodiment 68, wherein the linker comprises “N” consecutive polyethylene glycol units, wherein N is between 30 and 70.
70. The CpG-amphiphile and a coronavirus antigen, or a nucleic acid sequence encoding the coronavirus antigen for use according to embodiment 69, wherein the linker comprises “N” consecutive polyethylene glycol units, wherein N is between 40 and 60.
71. The CpG-amphiphile and a coronavirus antigen, or a nucleic acid sequence encoding the coronavirus antigen for use according to embodiment 70, wherein the linker comprises “N” consecutive polyethylene glycol units, wherein N is between 45 and 55.
72. The CpG-amphiphile and a coronavirus antigen, or a nucleic acid sequence encoding the coronavirus antigen for use according to embodiment 71, wherein the linker comprises 48 consecutive polyethylene glycol units.
73. The CpG-amphiphile and a coronavirus antigen, or a nucleic acid sequence encoding the coronavirus antigen for use according to any one of embodiments 2 or 59-72, wherein the lipid is a diacyl lipid.
74. The CpG-amphiphile and a coronavirus antigen, or a nucleic acid sequence encoding the coronavirus antigen for use according to embodiment 73, wherein the diacyl lipid has the following structure:

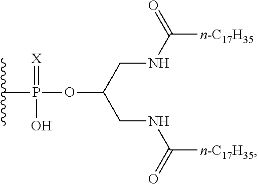

or a salt thereof,
wherein X is O or S.
75. The CpG-amphiphile and a coronavirus antigen, or a nucleic acid sequence encoding the coronavirus antigen for use according to any one of embodiments 2 or 59-74 wherein the CpG sequence comprises the nucleotide sequence 5′-TCGTCGTTTTGTCGTTTTGTCGTT-3′ (SEQ ID NO:1).
76. The CpG-amphiphile and a coronavirus antigen, or a nucleic acid sequence encoding the coronavirus antigen for use according to any one of embodiments 2 or 59-75, wherein the CpG sequence comprises the nucleotide sequence of 5′-TCCATGACGTTCCTGACGTT-3′ (SEQ ID NO: 2).
77. The CpG-amphiphile and a coronavirus antigen, or a nucleic acid sequence encoding the coronavirus antigen for use according to embodiment 75 or embodiment 76, wherein all internucleoside groups connecting the nucleosides in the CpG sequence are phosphorothioates.
78. The CpG-amphiphile and a coronavirus antigen, or a nucleic acid sequence encoding the coronavirus antigen for use according to any one of embodiments 2 or 59-77, wherein the coronavirus spike protein or peptide thereof is a SARS-CoV-2 spike protein or peptide thereof.
79. The CpG-amphiphile and a coronavirus antigen, or a nucleic acid sequence encoding the coronavirus antigen for use according to any one of embodiments 2 or 59-78, wherein the peptide of the coronavirus spike protein is a receptor binding domain that specifically binds angiotensin-converting enzyme 2 (ACE2).
80. The CpG-amphiphile and a coronavirus antigen, or a nucleic acid sequence encoding the coronavirus antigen for use according to any one of embodiments 2 or 59-79, wherein the peptide of the coronavirus spike protein comprises a polypeptide sequence having at least 90% sequence identity to:

(SEQ ID NO: 3)
RVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPA
TVCGPKKSTNLVKNKCVNFNFNGLTGTGVLTESNKKFLPFQQFGRDIADTT
DAVRDPQTLEILDITPCS.

81. The CpG-amphiphile and a coronavirus antigen, or a nucleic acid sequence encoding the coronavirus antigen for use according to embodiment 80, wherein the peptide of the coronavirus spike protein comprises the polypeptide sequence of:

(SEQ ID NO: 3)
RVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPA
TVCGPKKSTNLVKNKCVNFNFNGLTGTGVLTESNKKFLPFQQFGRDIADTT
DAVRDPQTLEILDITPCS.

82. The CpG-amphiphile and a coronavirus antigen, or a nucleic acid sequence encoding the coronavirus antigen for use according to any one of embodiments 2 or 61-79, wherein the coronavirus antigen is a coronavirus nucleocapsid protein or a peptide thereof.
83. The CpG-amphiphile and a coronavirus antigen, or a nucleic acid sequence encoding the coronavirus antigen for use according to embodiment 82, wherein the coronavirus nucleocapsid protein antigen comprises a polypeptide sequence having at least 90% sequence identity to:

(SEQ ID NO: 68)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWWVATEGALNTPKDHIGTR
NPANNAAIVLQLPQGTTLPKGFYAEGSRGGSQASSRSSSRSRNSSRNSTP
GSSRGTSPARMAGNGGDAALALLLLDRLNQLESKMSGKGQQQQGQTVTKK
SAAEASKKPRQKRTATKAYNVTQAFGRRGPEQTQGNFGDQELIRQGTDYK
HWPQIAQFAPSASAFFGMSRIGMEVTPSGTWLTYTGAIKLDDKDPNFKDQ
VILLNKHIDAYKTFPPTEPKKDKKKKADETQALPQRQKKQQTVTLLPAAD
LDDFSKQLQQSMSSADSTQA.

84. The CpG-amphiphile and a coronavirus antigen, or a nucleic acid sequence encoding the coronavirus antigen for use according to embodiment 82, wherein the coronavirus nucleocapsid protein antigen comprises the polypeptide sequence of:

(SEQ ID NO: 63)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWWVATEGALNTPKDHIGTR
NPANNAAIVLQLPQGTTLPKGFYAEGSRGGSQASSRSSSRSRNSSRNSTP
GSSRGTSPARMAGNGGDAALALLLLDRLNQLESKMSGKGQQQQGQTVTKK
SAAEASKKPRQKRTATKAYNVTQAFGRRGPEQTQGNFGDQELIRQGTDYK
HWPQIAQFAPSASAFFGMSRIGMEVTPSGTWLTYTGAIKLDDKDPNFKDQ
VILLNKHIDAYKTFPPTEPKKDKKKKADETQALPQRQKKQQTVTLLPAAD
LDDFSKQLQQSMSSADSTQAENLYFQGHHHHHH.

85. The CpG-amphiphile and a coronavirus antigen, or a nucleic acid sequence encoding the coronavirus antigen for use according to any one of embodiments 2 or 59-83, wherein the coronavirus antigen comprises one or more tags.
86. The CpG-amphiphile and a coronavirus antigen, or a nucleic acid sequence encoding the coronavirus antigen for use according to embodiment 85, wherein the tag is an Avi tag.
87. The CpG-amphiphile and a coronavirus antigen, or a nucleic acid sequence encoding the coronavirus antigen for use according to embodiment 85, wherein the tag is a histidine tag.
88. The CpG-amphiphile and a coronavirus antigen, or a nucleic acid sequence encoding the coronavirus antigen for use according to any one of embodiments 85-86, wherein the coronavirus antigen comprises an Avi tag and a histidine tag.
89. The CpG-amphiphile and a coronavirus antigen, or a nucleic acid sequence encoding the coronavirus antigen for use according to any one of embodiments 85-88, wherein the coronavirus antigen comprises a linker between the polypeptide sequence and the one or more tags.
90. The CpG-amphiphile and a coronavirus antigen, or a nucleic acid sequence encoding the coronavirus antigen for use according to any one of embodiments 2 and 59-89, wherein the coronavirus spike protein is to be administered.
91. The CpG-amphiphile and a coronavirus antigen, or a nucleic acid sequence encoding the coronavirus antigen for use according to embodiment 90, wherein a trimer of the coronavirus spike protein is to be administered.
92. The CpG-amphiphile and a coronavirus antigen, or a nucleic acid sequence encoding the coronavirus antigen for use according to embodiment 91, wherein the trimer is a trimer of a protein construct comprising the sequence:

(SEQ ID NO: 66)
VNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRD
LPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQT
SNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIP
IGAGICASYQTQTNSPRAAASVASQSIIAYTMSLGAENSVAYSNNSIAIP
TNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKR
SFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNV
LYENQKLIANQFNSAIGKIQDSLSSTASALGKLQDVVNQNAQALNTLVKQ
LSSNFGAISSVLNDILSRLDKVEAEVQIDRLITGRLQSLQTYVTQQLIRA
AEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPGG
GSGGGSHHHHHHHHHH.

93. The CpG-amphiphile and a coronavirus antigen, or a nucleic acid sequence encoding the coronavirus antigen for use according to any one of embodiments 2 and 59-92, wherein a coronavirus spike protein, or a peptide thereof, and a coronavirus nucleocapsid protein, or a peptide thereof, are to be administered.
94. The CpG-amphiphile and a coronavirus antigen, or a nucleic acid sequence encoding the coronavirus antigen for use according to embodiment 93, wherein a trimer of a coronavirus spike protein construct comprising the sequence:

(SEQ ID NO: 66)
VNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRD
LPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQT
SNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIP
IGAGICASYQTQTNSPRAAASVASQSIIAYTMSLGAENSVAYSNNSIAIP
TNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKR
SFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNV
LYENQKLIANQFNSAIGKIQDSLSSTASALGKLQDVVNQNAQALNTLVKQ
LSSNFGAISSVLNDILSRLDKVEAEVQIDRLITGRLQSLQTYVTQQLIRA
AEIRASANLAATKMSECVLGQSKRVDFCGKGYHLMSFPQSAPHGWVFLHV
TYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPGG
GSGGGSHHHHHHHHHH,

and a coronavirus nucleocapsid protein construct having the polypeptide sequence of: MSDNGPQNQRNAPRITFGGPSDSTGSNQNGERSGARSKQRRPQGLPNNTASWFTALTQHGKEDL KFPRGQGVPINTNSSPDDQIGYYRRATRRIRGGDGKMKDLSPRWYFYYLGTGPEAGLPYGANKDGII WVATEGALNTPKDHIGTRNPANNAAIVLQLPQGTTLPKGFYAEGSRGGSQASSRSSSRSRNSSRNS TPGSSRGTSPARMAGNGGDAALALLLLDRLNQLESKMSGKGQQQQGQTVTKKSAAEASKKPRQKR TATKAYNVTQAFGRRGPEQTQGNFGDQELIRQGTDYKHWPQIAQFAPSASAFFGMSRIGMEVTPSG TWLTYTGAIKLDDKDPNFKDQVILLNKHIDAYKTFPPTEPKKDKKKKADETQALPQRQKKQQTVTLLP AADLDDFSKQLQQSMSSADSTQAENLYFQGHHHHHH (SEQ ID NO:63) are to be administered.
95. The CpG-amphiphile and a coronavirus antigen, or a nucleic acid sequence encoding the coronavirus antigen for use according to any one of embodiments 2 and 59-94, wherein an mRNA encoding the coronavirus antigen is administered.
96. The CpG-amphiphile and a coronavirus antigen, or a nucleic acid sequence encoding the coronavirus antigen for use according to any one of embodiments 2 and 59-95, wherein the CpG-amphiphile and the coronavirus antigen or nucleic acid sequence encoding the same are to be administered concurrently.
97. The CpG-amphiphile and a coronavirus antigen, or a nucleic acid sequence encoding the coronavirus antigen for use according to any one of embodiments 2 and 59-96, wherein the CpG-amphiphile and the coronavirus antigen, or nucleic acid sequence enclosing the same are to be administered sequentially.
98. The CpG-amphiphile and a coronavirus antigen, or a nucleic acid sequence encoding the coronavirus antigen for use according to embodiment 97, wherein the CpG-amphiphile is to be administered first, followed by administering of the coronavirus antigen or nucleic acid sequence encoding the same.
98. The CpG-amphiphile and a coronavirus antigen, or a nucleic acid sequence encoding the coronavirus antigen for use according to embodiment 97, wherein said the coronavirus antigen or nucleic acid sequence encoding the same is to be administered first, followed by administering of CpG-amphiphile.
99. The CpG-amphiphile and a coronavirus antigen, or a nucleic acid sequence encoding the coronavirus antigen for use according to any one of embodiments 2 and 59-99, wherein a second adjuvant is to be administered to the subject.
100. The CpG-amphiphile and a coronavirus antigen, or a nucleic acid sequence encoding the coronavirus antigen for use according to any one of embodiments 2 and 59-99, wherein a coronavirus vaccine is to be administered to the subject as a prime or a boost.
101. The CpG-amphiphile and a coronavirus antigen, or a nucleic acid sequence encoding the coronavirus antigen for use according to any one of embodiments 2 and 59-100, wherein the CpG-amphiphile is to be administered subcutaneously, intranasally, intratracheally, or by inhalation during mechanical ventilation.
102. The CpG-amphiphile and a coronavirus antigen, or a nucleic acid sequence encoding the coronavirus antigen for use according to embodiment 101, wherein the CpG-amphiphile is to be administered subcutaneously.
103. The CpG-amphiphile and a coronavirus antigen, or a nucleic acid sequence encoding the coronavirus antigen for use according to any one of embodiments 2 and 59-102, wherein the coronavirus antigen is to be administered subcutaneously, intranasally, intratracheally, or by inhalation during mechanical ventilation.
104. The CpG-amphiphile and a coronavirus antigen, or a nucleic acid sequence encoding the coronavirus antigen for use according to any one of embodiments 2 and 59-103, wherein the subject is a mammal.
105. The CpG-amphiphile and a coronavirus antigen, or a nucleic acid sequence encoding the coronavirus antigen for use according to embodiment 104, wherein the subject is a human.

### EXAMPLES

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1: Inducing an Immune Response in Mice

Stock solutions of the free CpG (CpG 1826), CpG-amphiphile (aCpG 1826) were made by resuspending the CpG-amphiphile in limulus amebocyte lysate (LAL) H₂O. Final injections were diluted with 1× phosphate buffered saline (PBS) such that the final concentration of CpG was 1 nmol/100 μL injection. The SARS-CoV2 Spike S1 RBD protein (SEQ ID NO: 3) (Table 1) stock solutions were dissolved in PBS at a concentration of 1.2 mg/ml. Final injections were diluted with 1× PBS to 10 μg/100 μL injection.

IFA (Incomplete Freund's Adjuvant) solutions were made using a 1:1 mix of 50 μL antigen suspended in PBS and 50 μL IFA, followed by pipetting up and down vigorously for 30 seconds. Alhydrogel 2% (10 mg/ml) solutions were made using a 1:9 mix of 10 μg antigen suspended in PBS and 100 μg alum, which is equivalent 10 μL. To this solution the antigen was added made up with 100 μL with PBS. The solution was mixed by pipetting vigorously for 5 min.

Immunizations were administered subcutaneously (SC) into the tail base of female C57Bl6 mice bilaterally with 50 μL per side. Booster doses was given at roughly 2-week intervals.

SC injections ensured that the vaccine was optimally delivered into lymph nodes via natural lymph drainage. Bi-weekly injections were determined to be optimal in immune response generation in previous mouse studies.

TABLE 1
Vaccine Components

| Vaccine | | | |
|---|---|---|---|
| Components | Sequence or Cat# | Source | Lot # |
| SARS-COV2 | 40592-V08H | SinoBio | MA14MA1904 |
| RBD, His | | | |
| CpG 1826 | 5′-ggt cca tga cgt tcc | InvivoGen | 4103-24T |
| | tga cgt t-3′ (SEQ ID | | |
| | NO: 2) | | |
| aCpG 1826 | 5′-(Diacyl lipid) ggt | Avecia | S17-048- |
| | cca tga cgt tcc tga | | S3-B1 |
| | cgt t-3′ (SEQ ID NO: | | |
| | 2) | | |
| Alhydrogel | vac-alu-250 | InvivoGen | 1614532 |
| IFA | vac-ifa-10 | InvivoGen | IFA-41-01 |

Intracellular Stain (ICS) assays for TNFα and IFNγ were performed on peripheral blood mononuclear cells (PBMCs) 7 days after dosing. Cells were surface stained for CD4 and CD8 and sometimes CD3. For ICS #1, after the first dose, and ICS #2, after the second dose, PBMCs were activated for 5 hours (4 hours in the presence of Brefeldin A) with 1 μg/well of peptide (Table 2). C57Bl6 cells were re-stimulated with peptides that have a calculated affinity to db/Kb. Balb/c cells were re-stimulated with peptides that have a calculated affinity to Dd/Kd

TABLE 2
Re-Stimulation Peptides

| Re-stimulation | | | |
|---|---|---|---|
| Peptides | Sequence | Source | Lot # |
| H-2-Db1 | YSVLYNSASF | GenScript | U956FFC260- |
| | (SEQ ID NO: 39) | | 1-PE8358 |
| H-2-Db4/Dd4 | YQPYRVVVL | GenScript | U956FFC260- |
| | (SEQ ID NO: 40) | | 10-PE8364 |
| H-2-Db5/Kb8/ | VRFPNITNL | GenScript | U956FFC260- |
| Dd5/Kd5 | (SEQ ID NO: 41) | | 13-PE8366 |
| H-2-Kb2 | FNATRFASV | GenScript | U956FFC260- |
| | (SEQ ID NO: 42) | | 19-PE8370 |
| H-2-Kb3 | KIADYNYKL | GenScript | U956FFC260- |
| | (SEQ ID NO: 43) | | 22-PE8372 |
| H-2-Kb7/Dd7 | VSPTKLNDL | GenScript | U956FFC260- |
| | (SEQ ID NO: 44) | | 34-PE8380 |
| H-2-Dd1 | VCGPKKSTNL | GenScript | U956FFC260- |
| | (SEQ ID NO: 45) | | 37-PE8382 |
| H-2-Kd2 | SYGFQPTNGV | GenScript | U956FFC260- |
| | (SEQ ID NO: 46) | | 49-PE8390 |
| H-2-Kd3 | VYAWNRKRI | GenScript | U956FFC260- |
| | (SEQ ID NO: 47) | | 52-PE8392 |
| H-2-Kd4 | SFVIRGDEV | GenScript | U956FFC260- |
| | (SEQ ID NO: 48) | | 55-PE8394 |

Cytometric Bead Array (CBA) analysis was performed for IL2, IL4, IL6, IL10, IL17, TNFα and IFNγ was performed on splenocytes 7 days after dose administration. For CBA #1, after dose one, PBMCs were activated overnight with 5 μg/well of peptide (Table 2). For CBA #2, after the second dose, PBMCs were activated overnight with 0.42 μg/well of PepMix™ SARS-CoV-2 Spike Glycoprotein (315 peptides each at 0.42 μg/well) (Table 3). For CBA #3, after dose three, PBMCs were activated overnight with 1 μg/well of PepMix™ SARS-CoV-2 Spike Glycoprotein (315 peptides each at 1 μg/well) (Table 3).

TABLE 3
Re-Stimulation PepMix

| Re-stimulation | | | |
|---|---|---|---|
| Peptides | Sequence | Source | Lot # |
| PepMix ™ | 315 15mers spanning | JPT | 42669FRa-1 |
| SARS-CoV-2 Spike | Spike Protein Sequence, | | (158 peptides) |
| Glycoprotein | overlap 11aa | | 42669FRa-2 |
| | | | (157 peptides) |

SARS-CoV2 specific serum antibody enzyme-linked immunosorbent assays (ELISA) were performed on mouse serum 7 days after each dose, to detect any RBD-specific antibody response. Whole blood was spun down using Ser-gel tubes (NC9436363, Fisher Scientific). Serum was either used fresh or stored at −80° C. until used. 96-well plates were coated with 200 ng/100 μl (2 μg/ml) of CoV2 RBD protein (Z03483, GenScript) overnight at 4° C. Then plates were pre-blocked with PBS+2% BSA for 2 h at room temperature. Mouse serum was diluted 1:10 and then serially diluted (1:4→8 concentrations) in a dummy plate. Samples were transferred to the ELISA plate and incubated for 2 h at room temperature. As positive control, two antibodies were used: Creative Diagnostics (CABT-CS035; clone 211184): mouse aRBD Mab and MyBioSource (MBS434247): mouse aRBD Mab. Plates were washed 4 times with washing buffer (BioLegend 4211601). As secondary detection Abs, the following were used at 1:2000 in PBS+ and incubated for 1 h at room temperature (RT): In initial experiments, AffiniPure Rabbit Anti-Mouse IgG+IgM (H+L) HRP (315-035-048, Jackson ImmunoResearch) was used, but in subsequent experiments, AffiniPure Rabbit Anti-Mouse IgM (μ chain) HRP (315-035-049, Jackson ImmunoResearch) and AffiniPure Rabbit Anti-Mouse IgG (Fcγ) HRP (315-035-046, Jackson ImmunoResearch) were used. Plates were washed 4 times with washing buffer. The reaction was visualized by addition of substrate 3,3′,5,5′-Tetramethylbenzidine (TMB) for 10 min at RT and stopped by H2SO4 (1 N). The absorbance at 450 nm was measured by an ELISA plate reader.

Anti-His tag ELISA assays were performed to determine if some of the immune response generated upon vaccination with the RBD-His protein construct is directed against the His-tag rather than the Spike RBD itself. Plates were coated with irrelevant protein, which was His-tagged (PDL1-His). Sera were only tested at undiluted concentrations. As secondary antibody, Rabbit Anti-Mouse IgG (Fcγ) HRP (315-035-046, was used. As positive control, THE His Tag Antibody [HRP] (A00612, GenScript) was used. Otherwise, ELISAs were performed as described above.

Neutralizing Antibody Assays were performed using the SARS-CoV-2 Surrogate Virus Neutralization Test Kit (Cat #L00847) from GenScript. The horseradish peroxidase (HRP)-RBD was prepared by conjugated RBD 1 in 1000 with HRP dilution buffer. For preparation of a whole plate, 5994 μl buffer+6 μl HRP-conjugated RBD was used. 55 μl of diluted HRP-RBD was transferred to a fresh plate, referred to as the “serum incubation plate”. Serially diluted sample sera were placed in a separate V-bottom plate, referred to as “serial dilution plate.” To the first row, 20 μl of undiluted serum was added. 8 μl of undiluted serum was then transferred to the subsequent wells, which contain 24 μl of PBS (1 in 4 serial dilution). Dilute sample sera (as well as positive and negative controls) were diluted 1 in 10 with sample dilution buffer. This was done by adding 54 μl buffer to a fresh plate, referred to as “final serum dilution plate” and transferring 6 μl of the serially diluted serum to that plate. 55 μL of finally diluted serum (and controls) were transferred to the serum incubation plate, which already contained 55 μl of HRP-conjugated RBD, which resulted in a 1:1 mix of RBD and serum, for a final dilution of 1 in 20, with subsequent 1:4 serial dilution.

The serum incubation plate was incubated at 37° C. for 30 min. 100 μL of the incubated serum-RBD mixture was transferred to the ACE2-precoated assay plates. The plates were covered with the provided sealer and incubated at 37° C. for 15 min. The plates were then washed 4 times with 200 μl of 1× Wash Solution, which consisted of 20× Wash Buffer diluted with deionized water. 100 μl of TMB Solution was added to each well and incubated at room temperature for 10 min. To quench the reaction, 50 μl of Stop Solution was added to each well. Absorbance was read immediately at 450 nm.

ELI-spot analysis for IFNγ was performed on splenocytes 9 days after administration of the fourth dose. Splenocytes (0.5×10⁶ cells/well) were activated with either 5 μg/well Peptides (Table 2 or Table 4) or 0.84 μg/well PepMix (Table 3). IFNγ plates were stimulated overnight.

TABLE 4
Re-Stimulation Peptides (2^{nd} Batch)

| Re- | | | |
|---|---|---|---|
| stimulation | | | |
| Peptides | Sequence | Source | Lot # |
| CoV2 #1 | VNFNFNGL | GenScript | U842NFE140- |
| | (SEQ ID NO: 49) | | 0/PE2815 |
| CoV2 #2 | KCYGVSPTKL | GenScript | U842NFE140- |
| | (SEQ ID NO: 50) | | 4/PE2818 |
| CoV2 #3 | CYGVSPTKL | GenScript | U842NFE140- |
| | (SEQ ID NO: 51) | | 7/PE2821 |
| CoV2 #4 | CYGVSATKL | GenScript | U842NFE140- |
| | (SEQ ID NO: 52) | | 10/PE2824 |
| CoV2 #5 | YGVSPTKL | GenScript | U842NFE140- |
| | (SEQ ID NO: 53) | | 13/PE2827 |
| CoV-Db2 | SKVGGNYNYL | GenScript | U842NFE140- |
| | (SEQ ID NO: 54) | | 16/PE4296 |
| CoV-Db3 | VIAWNSNNL | GenScript | U842NFE140- |
| | (SEQ ID NO: 55) | | 37/PE4338 |
| CoV-Kb1 | ESIVRFPNI | GenScript | U842NFE140- |
| | (SEQ ID NO: 56) | | 22/PE4323 |
| CoV-Kb4 | VVVLSFELL | GenScript | U842NFE140- |
| | (SEQ ID NO: 57) | | 25/PE4326 |
| CoV-Kb5 | GNYNYLYRL | GenScript | U842NFE140- |
| | (SEQ ID NO: 58) | | 28/PE4329 |
| CoV-Kb6/ | VGYQPYRVV | GenScript | U842NFE140- |
| CoV-Dd6 | (SEQ ID NO: 59) | | 31/PE4332 |
| CoV-Dd2 | YNSASFSTF | GenScript | U842NFE140- |
| | (SEQ ID NO: 60) | | 34/PE4335 |
| CoV-Dd3 | IAPGQTGKI | GenScript | U842NFE140- |
| | (SEQ ID NO: 61) | | 19/PE4320 |

Pseudovirus Neutralization Assays were performed by GenScript according to their procedures and protocols (SC1993-8). All 60 mouse samples of post dose 4 serum were sent to GenScript on dry ice, along with 22 human samples.

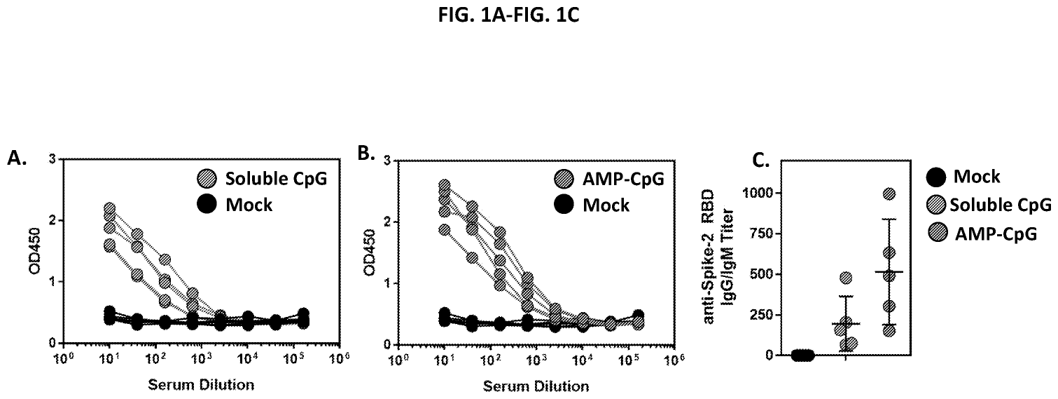

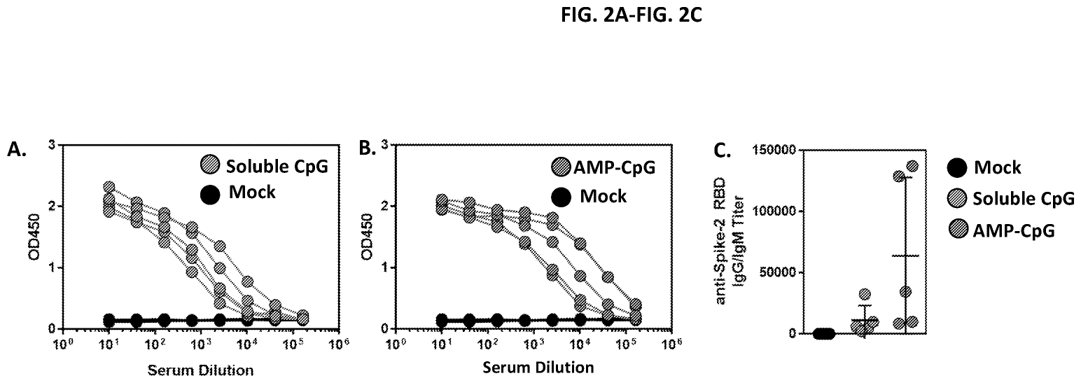

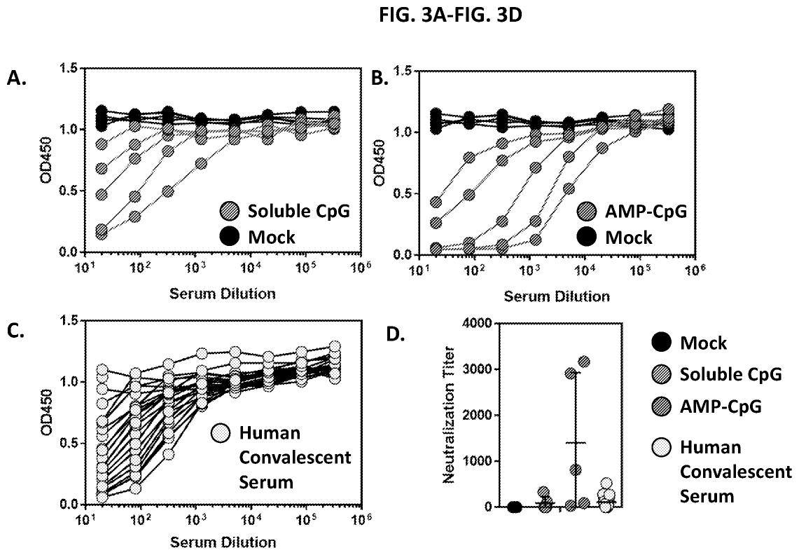

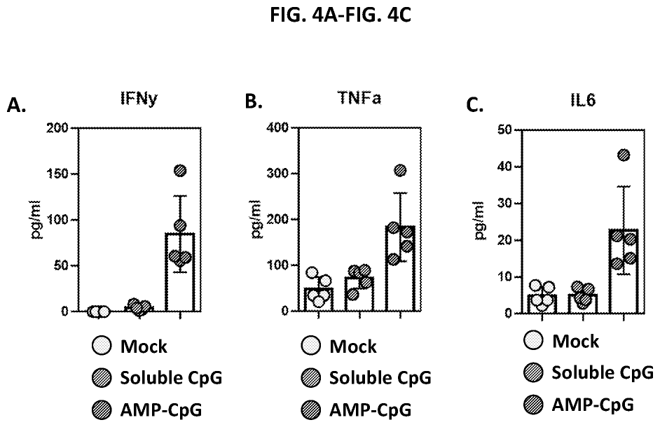

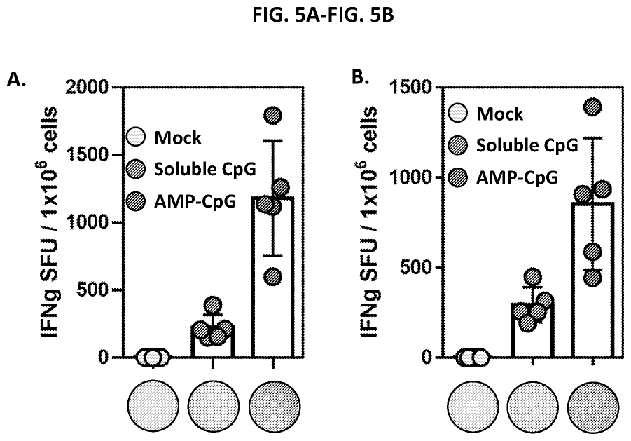

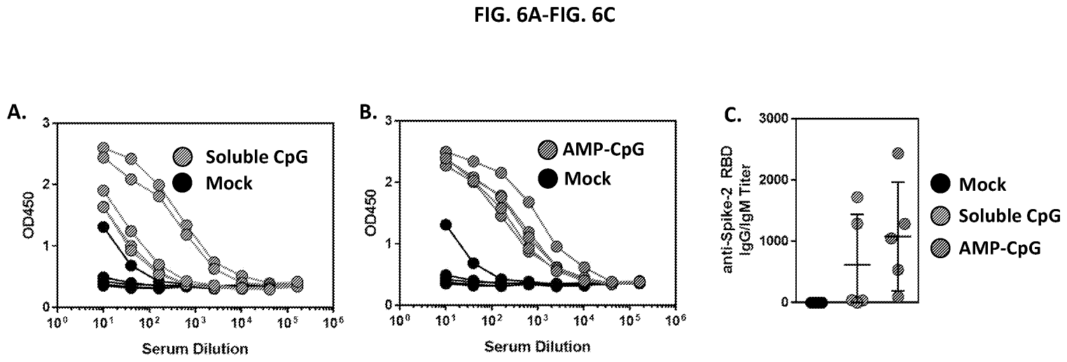

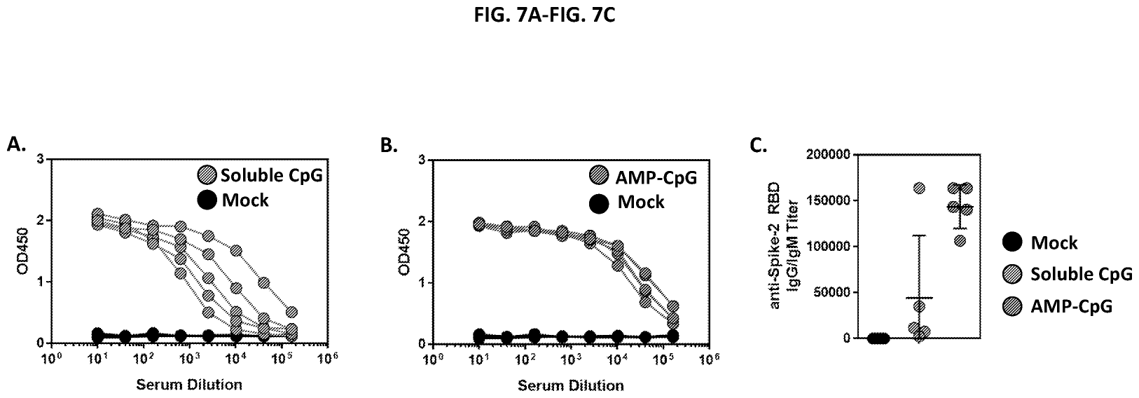

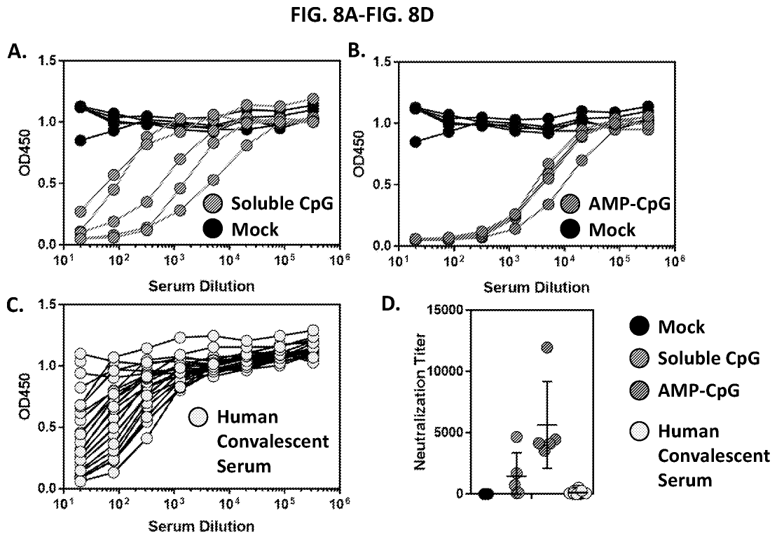

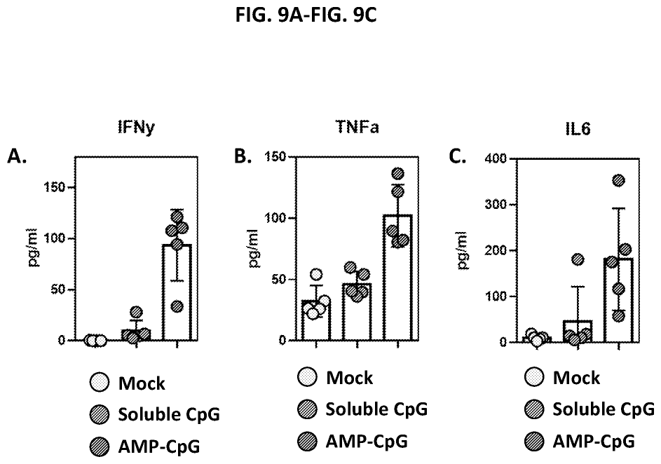

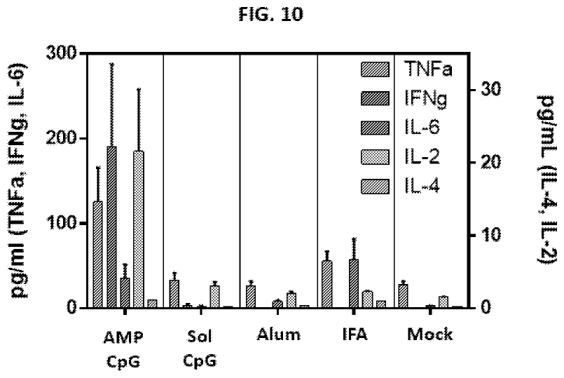

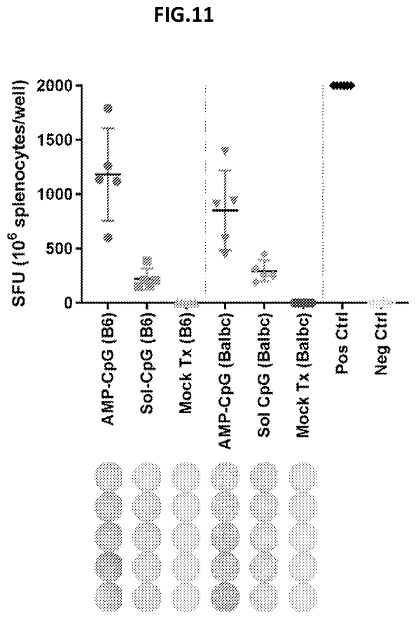

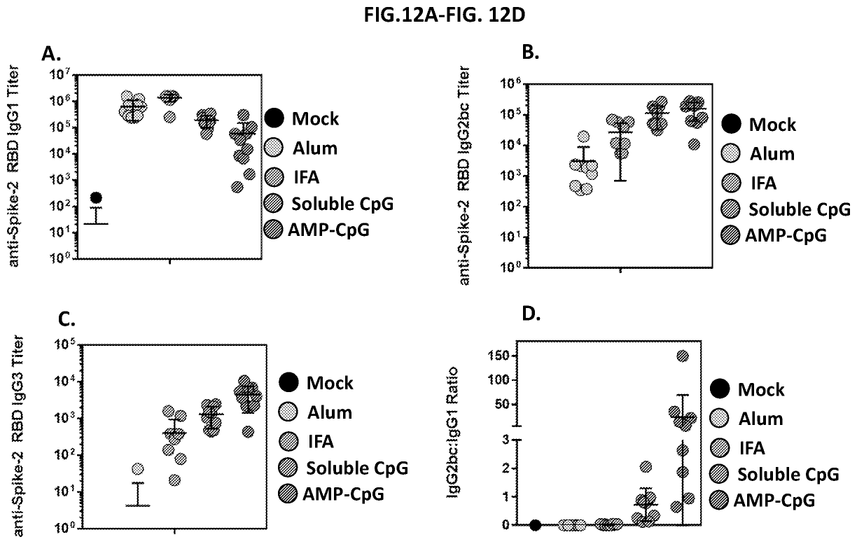

In order to induce an immune response, either C57Bl6 or Balb/C mice were administered a pharmaceutical composition formulated for a vaccine including 10 μg of a coronavirus spike protein peptide (SEQ ID NO: 3) and 8 μg equivalent of either a soluble CpG or a CpG-amphiphile. The mice were administered a first dose on day 0 and a second dose on day 14. On day 21, the amount of serum IgG/IgM antibodies was measured using a serum ELISA assay (FIG. 1A-FIG. 1C and FIG. 6A-FIG. 6C). Another dose of the CpG-amphiphile and the coronavirus spike protein peptide was administered on day 28. The amount of IgG/IgM antibodies for the mice that were administered the soluble CpG or the CpG-amphiphile was measured after 35 days using a serum ELISA assay (FIG. 2A-FIG. 2C and FIG. 7A-FIG. 7C) in comparison to a control. Also, after 35 days and three doses a peripheral blood mononuclear cell cytokine assay was performed to identify the amount of neutralizing antibodies present which block the interaction between the coronavirus spike protein and the ACE2 receptor for C57Bl6 mice (FIG. 3A and FIG. 3B) and Balb/C mice (FIG. 8A and FIG. 8B) in comparison to the concentration of neutralizing antibodies in human convalescent serum, from a patient having had a COVID-19 infection (FIG. 3C and FIG. 3D and FIG. 8C and FIG. 8D). Additionally, on day 35, the polyfunctional cytokine secreting T-cell response was measured for IFNγ, TNFa, and IL6 for C57Bl6 mice (FIG. 4A-FIG. 4C) and Balb/C mice (FIG. 9A-FIG. 9C). Also, on day the C57Bl6 and Balb/C mice were evaluated for the amount IFNg present after receiving three doses of the coronavirus spike protein and the CpG (FIG. 5A and FIG. 5B). The concentration of TNFa, IFNg, IL-6, IL-2, and IL-4 present after 21 days and after receiving two doses is summarized in FIG. 10. ELISpot assays were performed on C57Bl6 and Balb/C mice after being administered four doses in order to assess the amount of splenocyte IFNγ, with the highest amount in those dosed with a CpG-amphiphile as shown in FIG. 11. The amount of IgG1 (FIG. 12A), IgG2bc (FIG. 12B), IgG3 (FIG. 12C), and the IgG2bc:IgG1 ratio (FIG. 12D) for C57Bl6 mice administered three doses was analyzed to understand the amount of Th1 and Th2 response. The ratio of IgG2bc:IgG1 in FIG. 12D shows that for, mice administered the CpG-amphiphile, the immune response skews strongly to Th1 and not Th2. This is advantageous because a Th2 response can be detrimental for SARS-CoV-2.

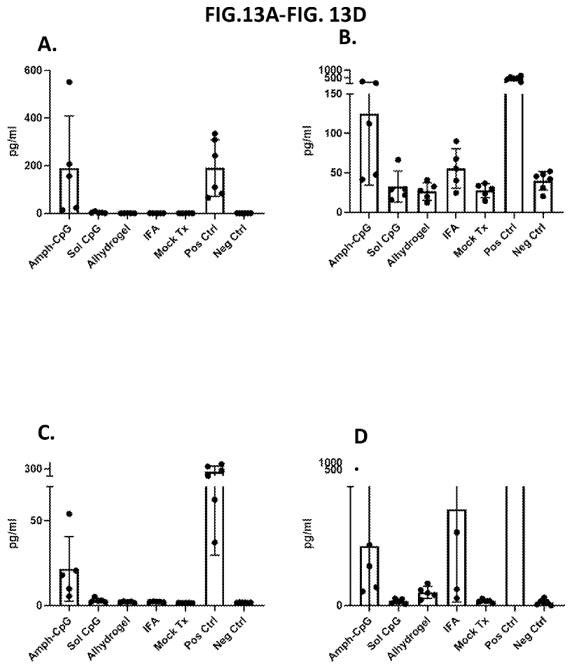

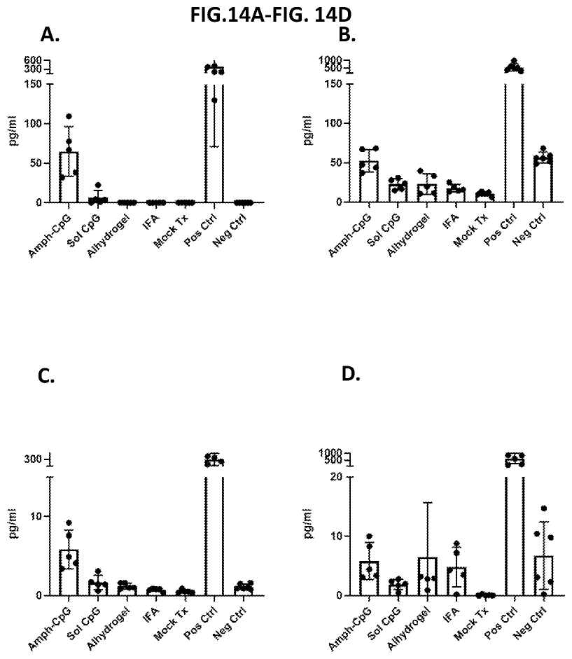

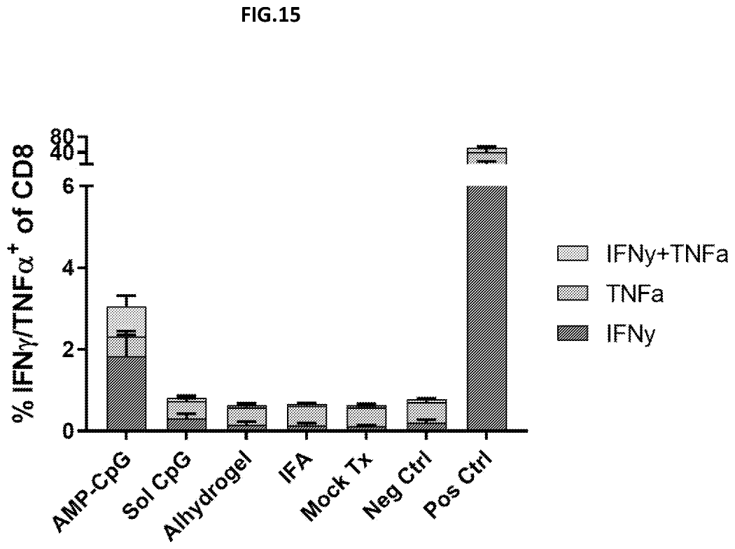

To compare how the CpG-amphiphile compares to other adjuvants, the amount of IFNγ, TNFa, IL-2, and IL-6 produced by mice which were administered two doses (FIGS. 13A-FIG. 13D) or three doses (FIG. 14A-FIG. 14D) of 10 μg of a coronavirus spike protein and 8 μg of either CpG-amphiphile, soluble CpG, Alhydrogel, IFA, or Mock Tx in comparison to a positive or negative control. The results showed the CpG-amphiphile yielded a superior immune response relative to the other tested adjuvants (FIG. 15).

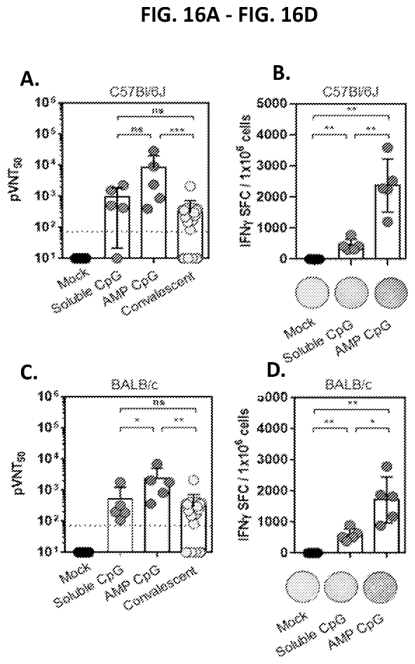

Further, female, 6 to 8-week-old C57BL/6J and BALB/c mice purchased from Jackson Laboratory (Bar Harbor, Me.) were injected with 1 nmol CpG (soluble CpG), 1 nmol lipid-conjugated CpG (AMP-CpG), or 100 μg Alum admixed with phosphate-buffered saline (PBS) only (adjuvant controls), or 1-10 μg of coronavirus spike protein (SEQ ID NO: 3) (Sino Biological, Cat: 40592-V08H or GenScript, Cat: Z03483). “Mock” groups received PBS alone. Injections (100 μL) were administered subcutaneously at the base of the tail (50 μL bilaterally) on days 0, 14, and 28 of the experiment. Blood samples were collected on days 7, 21, and 35. Mice were sacrificed on day 35 for lung harvest and collection of bronchoalveolar lavage (BAL) fluid. Only the set of mice (FIG. 16A-FIG. 16D) received a fourth dose on day 42 and samples were collected on day 49.

A pseudovirus neutralization assay was performed using the ACE2-HEK293 recombinant cell line (BPS Bioscience, Cat: 79951) or the control HEK293 cell line (ATCC) and the SARS-CoV2 Spike Pseudotyped Lentivirus (BPS Bioscience, Cat: 79942) containing the luciferase reporter gene and the SARS-CoV2 Spike envelope glycoproteins, thus specifically transducing ACE2-expressing cells. Mouse or human sera dilutions were performed in the Thaw Medium 1 (BPS Bioscience, Cat: 60187) in a 96-well white clear-bottom luminescence plate (Corning, Cat: 3610) and then pre-incubated with μL of virus for 30 minutes at RT. ACE2-HEK293 or control HEK293 cells (40 μL), containing 10,000 cells, were then added to the wells and incubated at 37° C. for 48 h. Control wells included ACE2-HEK293 cells or control HEK293 cells with the virus, but no sera, and provided the maximum transduction level and the background, respectively. Luciferase activity was detected by adding 70 μL of freshly prepared ONE-Step Luciferase reagent (BPS Bioscience, Cat: 60690) for 15 minutes at RT and luminescence was measured with a Synergy H1 Hybrid reader (BioTek). Pseudovirus neutralization data for the experiment was performed by GenScript (Nanjing, China) following the same protocol, but using in-house ACE2-HEK293 cells and Spike RBD-HRP recombinant protein. Pseudovirus neutralization titers at the half-maximal inhibitory dilution (pVNT₅₀) were calculated as the serum dilution at which RLU were reduced by 50% compared to RLU in virus control wells for C57Bl/6J mice (FIG. 16A) and BALB/c mice (FIG. 16C) that had been administered four doses of 10 μg of a coronavirus spike protein (SEQ ID NO: 3) in combination with 1 nmol soluble CpG or AMP-CpG compared to convalescent serum. The convalescent serum samples (n=7) and plasma samples (n=15) were obtained from patients who had recovered from SARS-CoV-2 infection (COVID-19) and were obtained from US Biolab (Rockville, Md.) and ALLCELLS (Alameda, Calif.), respectively.

Additionally, the amount of IFNγ produced by either C57Bl/6J mice (FIG. 16C) or BALB/c mice (FIG. 16D) was analyzed by individually collecting the mice spleens in RPMI 1640 media supplemented with 10% FBS and penicillin, streptomycin, nonessential amino acids, sodium pyruvate, and beta-mercaptoethanol (complete media) then processing into single cell suspensions and passing through a 70 μm nylon filter. Cell pellets were re-suspended in 3 mL of ACK lysis buffer (Quality Biological, Inc., Cat: 118156101) for 5 min on ice; then PBS was added to stop the reaction. The samples were centrifuged at 400×g for 5 min at 4° C. and cell pellets were re-suspended in complete media. ELISpot assays were performed using the Mouse IFN-γ ELISpot Set (BD, Cat: BD551083). 96-well ELISpot plates precoated with capture antibody overnight at 4° C. were blocked with complete media for 2 h at RT. 500,000 mouse splenocytes were plated into each well and stimulated overnight with 1 μg/peptide per well of Spike-derived overlapping peptides. The spots were developed based on manufacturer's instructions. PMA (50 ng/mL) and ionomycin (1 μM) were used as positive controls, and complete medium only as the negative control. Spots were scanned and quantified by an ImmunoSpot CTL reader. Initial assessments in C57BL/6J and BALB/C mice receiving immunization containing AMP-CpG produced a 10- to 30-fold higher pseudovirus neutralizing titer than natural antibody responses present in human convalescent serum (obtained from recovered COVID-19 patients; FIG. 16A and FIG. 16C), indicating the potential for AMP-CpG to produce neutralizing antibody responses more potent than natural immunity. By comparison, animals immunized with a dose-matched regimen containing unmodified (soluble) CpG produced neutralizing titers comparable to those observed in human convalescing patients. The results of splenocyte ELISpot assays showed that compared with soluble CpG, mice immunized with the coronavirus spike protein admixed with AMP-CpG elicited approximately 4-fold greater frequencies of antigen-specific functional T cells, producing IFNγ upon restimulation with coronavirus spike protein derived overlapping peptides (FIG. 16B and FIG. 16D).

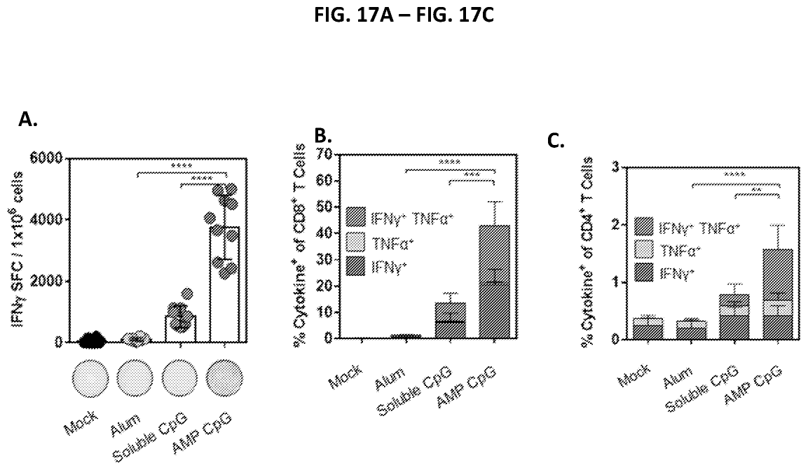

In the same manner, cytokine-producing cells in splenocytes and peripheral blood from C57BL/6J mice were determined. The number of IFNγ spot forming cells per 1×10⁶ splenocytes that were restimulated with overlapping coronavirus spike peptides were analyzed from C57BL/6J mice (n=10 per group) that received three doses of 10 μg of a coronavirus spike protein (SEQ ID NO: 3) in combination with 100 μg Alum, 1 nmol soluble CpG, or 1 nmol AMP-CpG (FIG. 17A). Mice immunized with the coronavirus spike protein in combination with AMP-CpG had substantially higher IFNγ spot forming cells than mice dosed coronavirus spike protein in combination with soluble CpG, alum, or mock (PBS).

### Example 2: Inducing a Humoral Immune Response in Mice

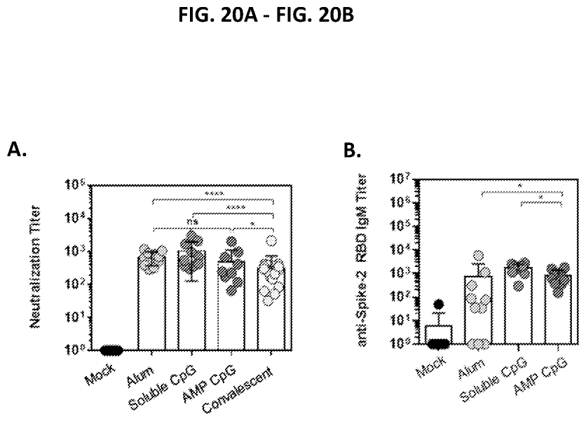

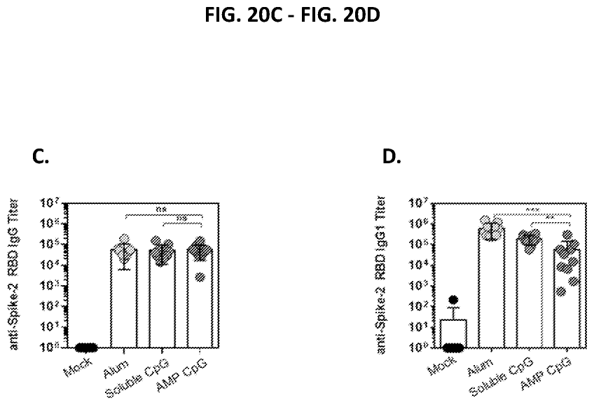

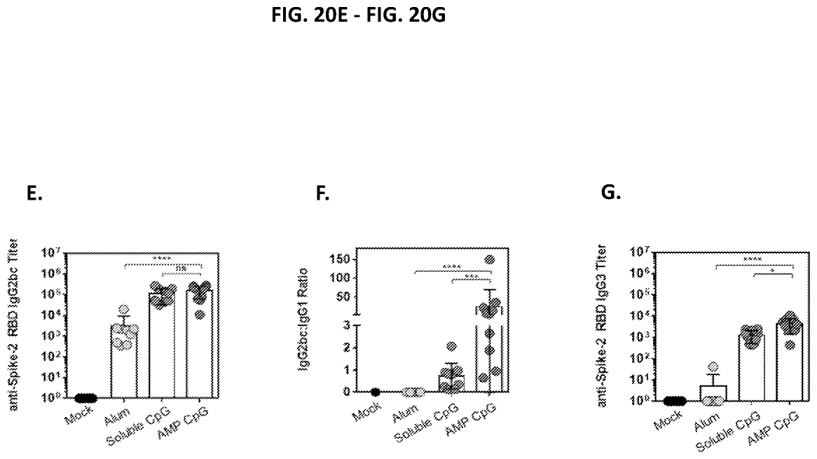

The humoral immune response induced in mice was determined for C57Bl/6J mice that were administered three doses of 10 μg of a coronavirus spike protein (SEQ ID NO: 3) in combination with 100 μg Alum, 1 nmol soluble CpG, or 1 nmol AMP-CpG. The humoral response was evaluated in terms of neutralization titer in comparison to convalescence serum (FIG. 20A), IgM (FIG. 20B), IgG (FIG. 20C), IgG1 (FIG. 20D), IgG2bc (FIG. 20E), the ratio of IgG2bc to IgG19 (FIG. 20F), and IgG3 (FIG. 20G) using either a pseudovirus neutralization assay or ELISA assay.

Neutralizing antibody responses to the coronavirus spike protein were measured through the inhibition of the coronavirus spike protein-ACE2 interaction in an ELISA-based surrogate assay. Results for serum collected on day 35 for cohorts of immunized C57BL/6J mice are shown in FIG. 20A. Comparable levels of neutralizing activity were induced in animals immunized with AMP-CpG, soluble CpG, and alum. Comparison with samples obtained from a cohort of convalescent humans showed that the vaccine-induced responses were significantly higher than those generated through response to natural infection.

Seven days after the initial immunization, all cohorts, except the control receiving mock immunization, showed robust coronavirus spike protein specific IgM responses (FIG. 20B); which underwent isotype switching to produce IgG responses with similar titer following subsequent boosting immunizations (FIG. 20C).

To assess Th1/Th2-bias in the coronavirus spike protein specific IgG response elicited through immunization, the IgG subclasses present were evaluated and showed that mice immunized with AMP-CpG or soluble CpG had significantly lower Th2 associated IgG1 titers (approximately 10-fold) than mice immunized with alum (FIG. 20D). The reverse was true for Th1 associated IgG2bc: titers were significantly higher (approximately 50-fold) for mice immunized with AMP-CpG or soluble CpG (FIG. 20E). The ratio of IgG2bc:IgG1 titer indicated a strong bias towards Th1 for AMP-CpG immunized animals, while soluble CpG and alum produced a balanced Th1/Th2 profile or Th2-dominant response, respectively (FIG. 20F). Further analysis showed AMP-CpG immunized animals produced significantly higher IgG3 titers than either soluble CpG (approximately 3-fold) or alum (>800-fold) treatment groups, which is consistent with the observed Th1-bias resulting from AMP-CpG immunization (FIG. 20G).

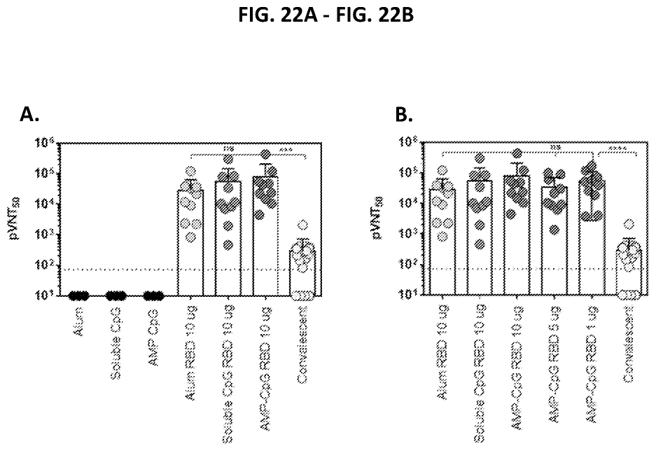

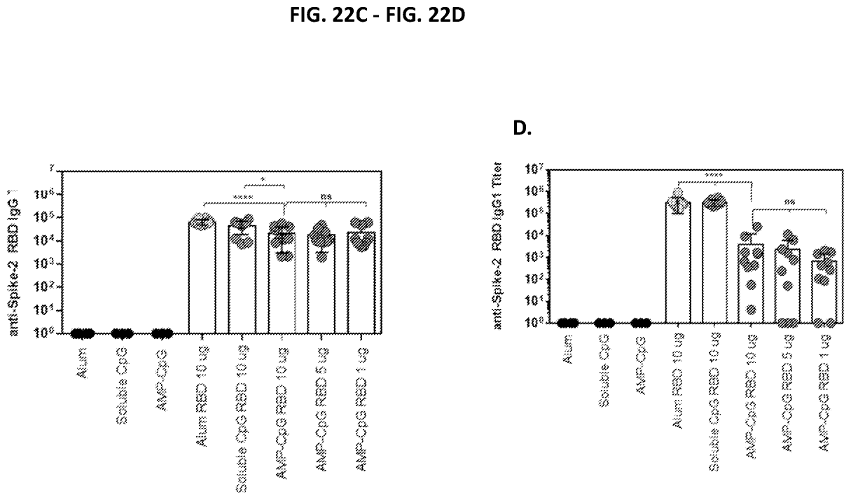

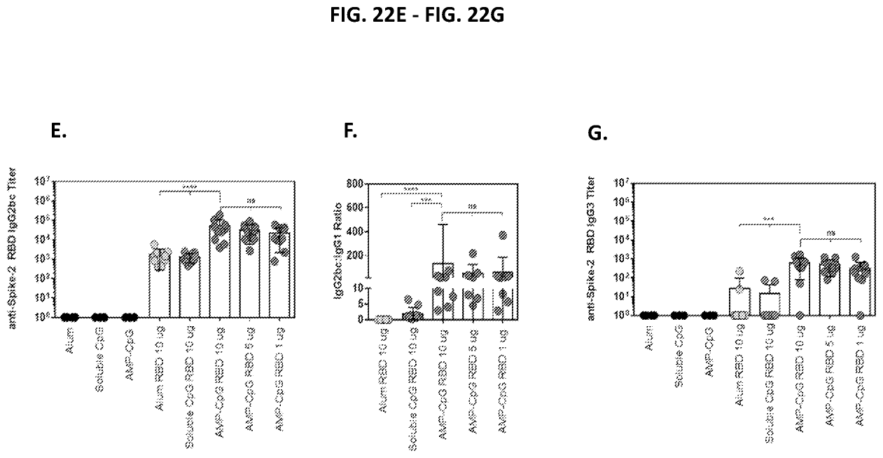

Additionally, the humoral response was assessed in serum for neutralization titer in comparison to convalescent serum (FIG. 22A), IgM (FIG. 22B), IgG (FIG. 22C), IgG1 (FIG. 22D), IgG2bc (FIG. 22E), the ratio of IgG2bc to IgG19 (FIG. 22F), and IgG3 (FIG. 22G) using either a pseudovirus neutralization assay or ELISA assay for C57Bl/6J mice that were administered three doses of only 10 μg of a coronavirus spike protein (SEQ ID NO: 3) in combination with only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3). On day 35 following repeat dose immunization, the induction of coronavirus-specific antibody responses among the AMP-CpG immunized animals at each specified coronavirus spike protein dose level was assessed and compared to responses generated by immunization with either soluble CpG or alum at the 10 μg dose. Neutralizing activity was assessed through measurement of pseudovirus neutralization titers at the half-maximal inhibitory dilution (pVNT₅₀) as described in Example 1. Similar levels of pseudovirus neutralization titers were observed for all treatment groups, at levels that were 265, 230, or 94-fold greater than those observed in convalescent human samples, for AMP-CpG, soluble CpG, and alum immunized mice, respectively (FIG. 22A). These levels were maintained in animals immunized with AMP-CpG at lower coronavirus spike protein dose levels with mean pVNT₅₀ at least 115-fold greater than those measured in recovering COVID-19 patients (FIG. 22B).

Total IgG titers were similar among the groups administered AMP-CpG, and these were reduced approximately 2-fold in comparison to titers measured among groups dosed with either soluble CpG or alum adjuvanted vaccines (FIG. 22C). Isotype analysis demonstrated similar trends to those initially observed in comparison at the 10 μg dose level (FIG. 22C). Alum and soluble CpG immunization produced significantly higher Th2-associated IgG1 titers (approximately 100-fold) compared to all coronavirus spike protein dose levels admixed with AMP-CpG (FIG. 22D). Th1-associated IgG2bc levels were elevated approximately 20-fold in all AMP-CpG immunized animals compared with soluble CpG and alum immunized groups, with no significant difference observed with reduced coronavirus spike protein dose level. These trends were further evident in the comparison of IgG2bc:IgG1 titer ratio (FIG. 22F), where AMP-CpG containing regimens induced highly Th1-dominant isotype profile (IgG2bc:IgG1>10), compared with more balanced and Th2-skewed responses in soluble CpG (IgG2bc:IgG1 approximately 2) and alum (IgG2bc:IgG1<1) vaccinated animals respectively. Finally, only animals immunized with AMP-CpG showed evidence of coronavirus spike protein specific IgG3 titers, with comparable levels detected among all coronavirus spike protein dose levels (approximately 500-fold over background). Further analysis showed AMP-CpG immunized animals produced significantly higher IgG3 titers than either soluble CpG (approximately 40-fold) or alum (>20-fold) treatment groups consistent with the observed Th1-bias resulting from AMP-CpG immunization (FIG. 22G). Together these data support AMP-CpG—enabling at least 10-fold dose sparing of coronavirus spike protein antigen for induction of neutralizing, high titer, and optimal Th1 profile antibody responses against coronavirus spike protein. While soluble CpG and alum induced marginally higher total IgG responses, these did not result in significant differences in neutralizing activity compared to AMP-CpG immunization. Alum and, to a lesser degree, soluble CpG responses were dominated by the Th2-associated IgG1 isotype raising the potential for a risk of toxicity in human translation based on prior outcomes in SARS and MERS vaccine development.

### Example 3: Inducing a Cellular Immune Response in Mice

Intracellular cytokine staining experiments were performed to assess the cellular immune response in mice administered a coronavirus spike protein in combination with an adjuvant, including alum, soluble CpG, and AMP-CpG. Peripheral blood cells that were collected 7 days after each booster dose and lung-resident leukocytes that were collected after the final booster dose were stimulated overnight with 1 μg of overlapping coronavirus spike peptide per well at 37° C., 5% CO2 in the presence of brefeldin A (Invitrogen, Cat: 00-4506-15) and monensin (BioLegend, Cat: 420701) as described in Example 1. Cells were stained with the following antibodies: PE anti-mouse IFNγ (BD, Cat: 554412), FITC anti-mouse TNFα (BD, Cat: 554418), APC-Cy™7 anti-mouse CD3 (BD, Cat: 560590), PE-Cy7 anti-mouse CD4⁺ (Invitrogen, Cat: 25-0041-82), and APC anti-mouse CD8a (eBioscience, Cat: 17-0081-83). PMA (50 ng/mL) and ionomycin (1 μM) were used as positive controls, and complete medium only as the negative control. Cells were permeabilized and fixed (Invitrogen, Cat: 00-5523-00). A LIVE/DEAD fixable (aqua) dead cell stain kit (Invitrogen, Cat: L34966) was used to evaluate viability of the cells during flow cytometry. Sample acquisition was performed on FACSCanto II (BD) and data analyzed with FlowJo V10 software (TreeStar).

The frequency of both IFNγ and TNFα (double-positive T-cells), only TNFα, and only IFNγ, in CD8⁺ T cells (FIG. 17B) or CD4⁺ T cells (FIG. 17C) were analyzed in peripheral blood cells from C57BL/6J mice that were administered three doses of 10 μg of a coronavirus spike protein (SEQ ID NO: 3) in combination with 100 μg Alum, 1 nmol soluble CpG, or 1 nmol AMP-CpG. Approximately 43% of CD8⁺ T cells derived from peripheral blood in AMP-CpG immunized mice were cytokine producing (IFNγ, TNFα, or double-positive T cells); in comparison, approximately 13% and <2% of CD8⁺ T cells were cytokine-producing for soluble CpG-immunized mice and alum-immunized mice, respectively (FIG. 17B). A similar trend was observed for CD4⁺ T cells, though percentages were relatively smaller: approximately 1.5% of T cells in peripheral blood from AMP-CpG immunized mice were cytokine-producing compared with <1% in CpG-immunized mice and <0.5% for alum-immunized mice and mock-immunized mice (FIG. 17C).

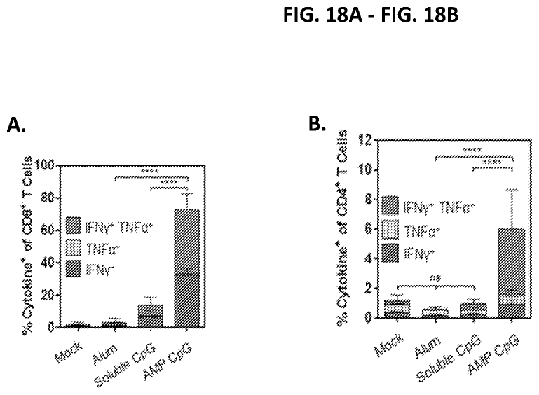

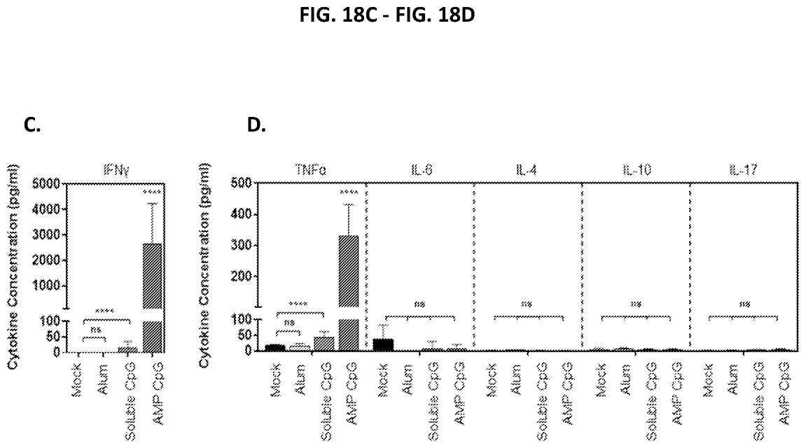

Likewise, to determine whether immunization could induce tissue resident T cell responses at a site of likely SARS-CoV-2 exposure the frequency of IFNγ and TNFα, only TNFα, and only IFNγ found in CD8⁺ T cells (FIG. 18A) or CD4⁺ T cells (FIG. 18B) perfuse lung tissue that was restimulated with overlapping coronavirus spike peptides in C57BL/6J mice that were administered three doses of pg of a coronavirus spike protein (SEQ ID NO: 3) in combination with 100 μg Alum, 1 nmol soluble CpG, or 1 nmol AMP-CpG was analyzed. T cells in the lung tissue had a greater proportion of the cytokine-producing cells than observed in peripheral blood. Observations in AMP-CpG immunized mice showed that approximately 73% of CD8⁺ T cells from perfused lung tissues were cytokine-producing, with approximately 40% exhibited polyfunctional secretion of both Th1 cytokines IFNγ and TNFα. By comparison, immunization with soluble CpG or alum induced >5-fold and >25-fold lower responses, respectively. Similar assessment of CD4⁺ T cells showed that only AMP-CpG immunized animals generated responses above background, with approximately 6% of CD4⁺ T cells producing IFNγ and/or TNFα, again exhibiting strong polyfunctional effector functionality, with the majority of these cells able to produce both IFNγ and TNFα upon antigen restimulation. These results support that the more potent lymph node action of AMP-CpG induces enhanced expansion of antigen-specific T cells with potentially beneficial tissue homing properties, establishing protective tissue resident cells at a primary site of initial viral exposure.

To more comprehensively understand the Th1/Th2/Th17 profile of the elicited T cell responses, a multiplexed cytokine assay was used to assess various cytokine concentrations from supernatants of cells collected from perfused lungs following restimulation with coronavirus spike overlapping peptides. Specifically, cytometric bead array flow cytometry was performed to determine cytokine production, including IFNγ (FIG. 18C), TFNα, IL-6, IL-4, IL-10, and IL17 (FIG. 18D), for C57BL/6J mice that were administered three doses of 10 μg of a coronavirus spike protein (SEQ ID NO: 3) in combination with 100 μg Alum, 1 nmol soluble CpG, or 1 nmol AMP-CpG. Lung-resident leukocytes (collected after the final booster dose) were activated overnight with overlapping coronavirus spike peptides at 1 μg/peptide per well (consisting of 315 peptides, derived from a peptide scan resulting in 15-mers with 11 amino acid overlap) (JPT, Cat: PM-WCPV-5 or GenScript, Cat: RP30020). Phorbol Myristate Acetate (PMA, 50 ng/mL) and ionomycin (1 μM) were used as positive controls, and complete medium only as the negative control. Culture supernatants were harvested and Th1/Th2 cytokine production was measured (CBA Mouse Th1/Th2/Th17 Cytokine Kit: BD, Cat: BDB560485). Briefly, bead populations with distinct fluorescence intensities that are coated with capture antibodies specific for various cytokines including IFNγ, TNFα, IL-4, IL-6, IL-10, and IL-17 were incubated with culture supernatants. The different cytokines in the sample were captured by their corresponding beads. The cytokine-captured beads were then mixed with phycoerythrin (PE)-conjugated detection antibodies. Following incubation, samples were washed, and fluorescent intensity of PE on the beads were measured and analyzed by flow cytometry (BD FACSCanto II). Mean fluorescent intensities (MFI) were calculated using FACSDiva software (BD) and protein concentrations were extrapolated using Microsoft Excel. AMP-CpG immunized mice exhibited a Th1 effector profile consistent with prior assessment by flow cytometry with IFNγ and TNFα concentrations that were significantly higher than cohorts immunized with the other adjuvants such as soluble CpG and alum or mock. The IFNγ concentration was at least 200-fold higher than observed with the other adjuvants or mock, and the TNFα concentration was at least 7-fold higher than the other adjuvants or mock (FIG. 18C). Concentrations of common Th2 or Th17 associated cytokines IL-4, IL-6, IL-10, and IL-17 were undetectable for all cohorts (FIG. 18D). These results further demonstrate the greatly enhanced potency and Th1-bias in T cells elicited through immunization with AMP-CpG immunization compared with either soluble CpG or alum.

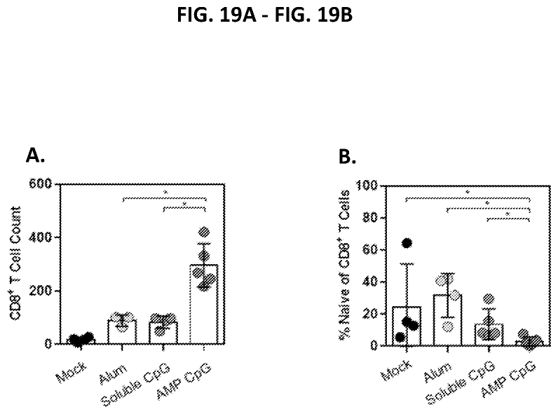

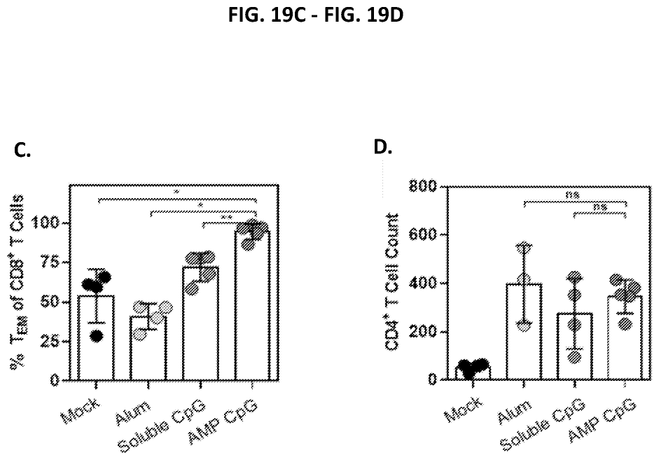

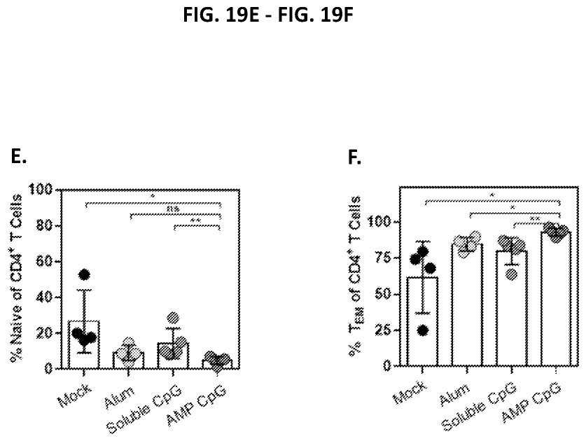

To further evaluate whether lung-resident T cell responses induced by immunization could localize into lung secretions, bronchoalveolar lavage (BAL) fluid was collected from C57BL/6J mice that were administered three doses of 10 μg of a coronavirus spike protein (SEQ ID NO: 3) in combination with 100 μg Alum, 1 nmol soluble CpG, or 1 nmol AMP-CpG. CD8⁺ (FIG. 19A) and CD4⁺ (FIG. 19D) T cell count, along with the percentage of naïve CD8⁺ (FIG. 19B) and naïve CD4⁺ (FIG. 19E) T-cells, and the percent of effector memory CD8⁺ (FIG. 19C) and CD4⁺ (FIG. 19F) T-cells were determined. Significantly more CD8⁺ T cells were found in BAL fluid of AMP-CpG immunized mice than other treatment groups (FIG. 19A). In addition, a significantly lower proportion of cells detected in the BAL collected from AMP-CpG immunized animals exhibited a naïve phenotype (CD44-, CD62L⁺; FIG. 19B) with a corresponding increase in the frequency of effector memory phenotype (T_{EM}; CD44⁺, CD62L−; FIG. 19C). The CD4⁺ T cell count was enhanced relative to mock treatment and generally similar across all treatment groups (FIG. 19D), but the AMP-CpG cohort showed evidence that a significantly greater proportion of the BAL-resident CD4⁺ T cells had differentiated from naïve to T_{EM} phenotype than in the other treatment groups (FIG. 19F). The improved numbers and phenotype of BAL-resident T cells present in AMP-CpG immunized animals demonstrate a greater potential for early immunological detection and control at the point of viral exposure.

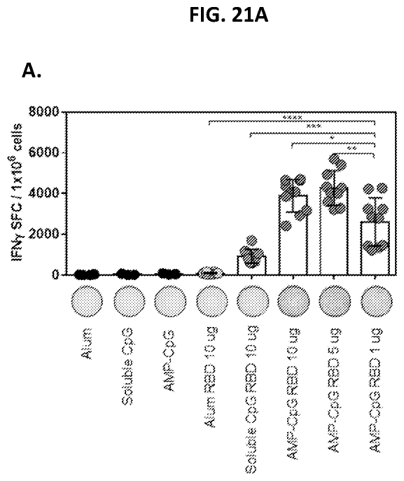

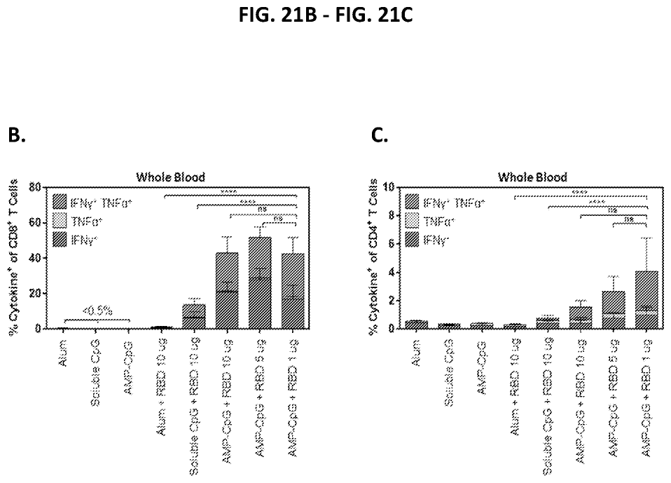

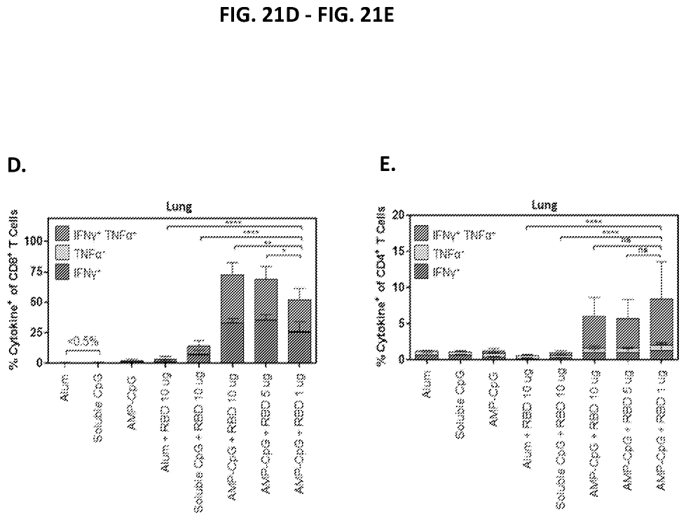

The T cell responses on day 35 in spleen, peripheral blood, and lung tissues were evaluated. The results showed that the number of IFNγ-producing cells in splenocytes collected from AMP-CpG immunized C57BL/6J mice tended to increase with antigen concentration, but, even at the lowest antigen dose admixed with AMP-CpG, the number of IFNγ-producing cells was significantly higher than observed in cohorts that received the highest antigen dose (10 μg) with either soluble CpG (approximately 4-fold) or alum (>30-fold) (FIG. 21A). Additionally, the frequency of cytokines, including IFNγ and TNFα, only TNFα, and only IFNγ, from CD8⁺ T-cells (FIG. 21B) and CD4⁺ T-cells (FIG. 21C) found in peripheral blood cells collected from C57BL/6J mice that were administered three doses of (from left to right) only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3) was determined. Likewise, the frequency of IFNγ and TNFα, only TNFα, and only IFNγ, of CD8⁺ T-cells (FIG. 21D) and CD4⁺ T-cells (FIG. 21E) found in perfused lung tissue cells, restimulated with overlapping coronavirus spike peptides, collected from C57BL/6J mice that were administered three doses of (from left to right) only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), and 1 nmol AMP-CpG and 1 μg of a coronavirus spike protein (SEQ ID NO: 3) were determined. In both peripheral blood (FIG. 21B and FIG. 21C) and lung tissue (FIG. 21D and FIG. 21E), the percent of CD8⁺ and CD4⁺ T cells producing cytokine was significantly higher for AMP-CpG treated mice at any concentration of antigen compared with the other adjuvants tested. Notably, no significant decrease in the frequency of cytokine-producing CD8⁺ or CD4⁺ T cells was observed in the peripheral blood of animals immunized with AMP-CpG admixed with antigen at 10, 5, or 1 μg dose levels as these were maintained at approximately 40-50% of CD8⁺ and 2-4% of CD4⁺ T cells. While a decreasing trend was observed in the frequency of lung-resident cytokine-producing CD8⁺ T cells in AMP-CpG immunized animals, even the 1 μg dose level produced frequencies >3-fold or >18-fold higher than animals immunized with soluble CpG or alum, respectively. This supports AMP-CpG enabling at least 10-fold dose sparing of the coronavirus spike protein.

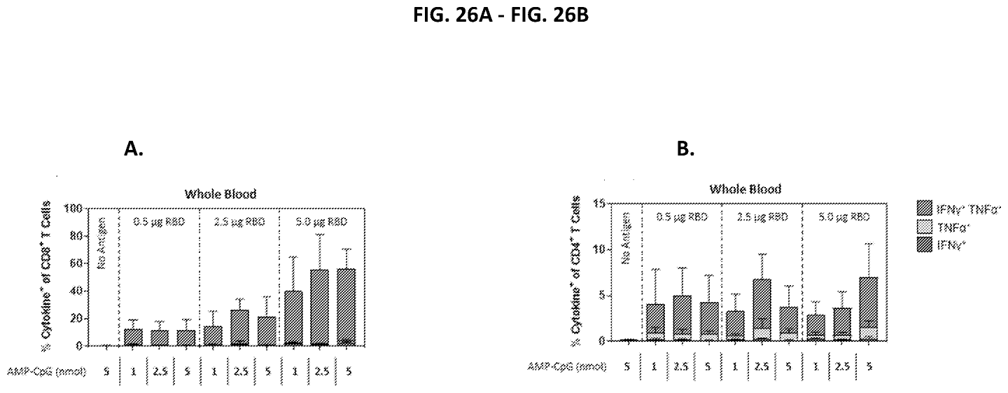

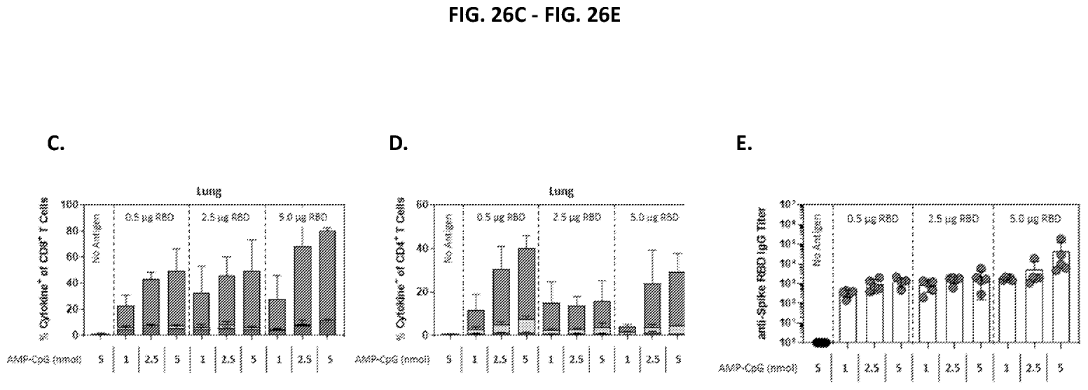

Two-dose vaccination with AMP-CpG-7909 elicits potent Spike RBD-specific cellular immunity in blood and lung, and humoral immunity in blood. C57Bl/6 mice (n=5 per group) were immunized on day 0 and 14 with 0.5, 1.0, or 5.0 ug Spike RBD protein admixed with 1.0, 2.5, or 5.0 nmol AMP-CpG, and T cell and IgG responses analyzed on day 21. Peripheral blood cells (FIG. 26A and FIG. 26B) or cells collected from perfused lungs (FIG. 26C and FIG. 26D) were restimulated with overlapping Spike RBD peptides and assayed by flow cytometry for intracellular cytokine production to detect antigen-specific T cell responses. Shown are frequencies of IFNγ, TNFα, and double-positive T cells among CD8⁺ (FIG. 26A and FIG. 26C) and CD4⁺ (FIG. 26B and FIG. 26D) T cells. Humoral responses specific to Spike RBD were assessed in serum from immunized animals by ELISA. Shown are endpoint titers for IgG on day 35 (FIG. 26E; n=5 mice per group). Values depicted are mean±standard deviation.

These results show that a two-dose regimen with AMP-CpG induces potent polyfunctional CD8 and CD4 T cell responses in blood and in the lungs.

### Example 4: Inducing an Immune Response in Aged Mice

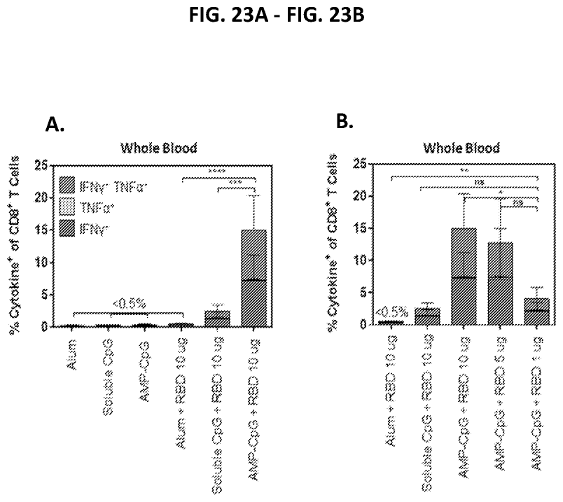

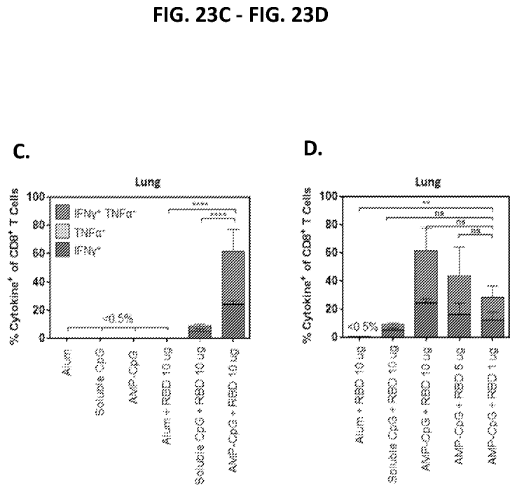

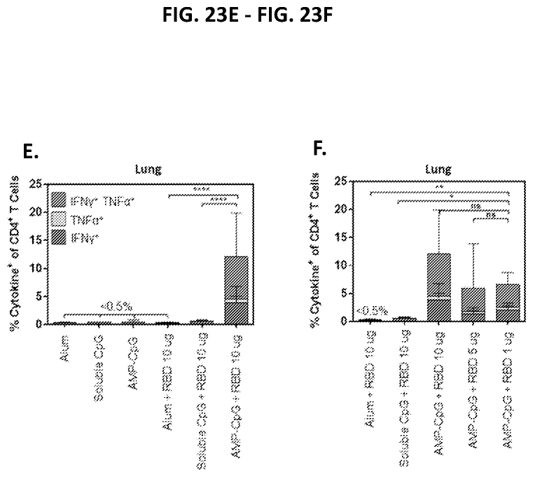

T cell responses in 37 week old C57BL/6J aged mice that were administered three doses of only 100 μg Alum, only 1 nmol soluble CpG, only 1 nmol AMP-CpG, 100 μg Alum and 10 μg of a coronavirus spike protein (SEQ ID NO: 3), 1 nmol soluble CpG and 10 μg of a coronavirus spike protein (SEQ ID NO: 3), 1 nmol AMP-CpG and 10 μg of a coronavirus spike protein (SEQ ID NO: 3), 1 nmol AMP-CpG and 5 μg of a coronavirus spike protein (SEQ ID NO: 3), or 1 nmol AMP-CpG and 1 μg of a coronavirus spike protein (SEQ ID NO: 3) mice were evaluated on days 21 and 35. Assessment on day 21 of cytokine-producing CD8⁺ T cells in peripheral blood following Spike-derived overlapping peptide restimulation showed that AMP-CpG induced potent responses (approximately 15% of CD8⁺ T cells), greatly outperforming soluble CpG (approximately 2.5% of CD8⁺ T cells), and alum (<0.5% of CD8⁺ T cells) (FIG. 23A). Although these responses were reduced approximately 2-fold compared to those observed in young healthy mice, they nonetheless exceeded those generated by the soluble CpG and alum comparators by 7- and 30-fold, respectively. AMP-CpG immunization further enabled comparable responses at 10 μg and 5 μg coronavirus spike protein doses, and although responses at 1 μg were decreased, these still exceeded the response observed for alum (20-fold) and were similar to those generated through immunization with soluble CpG at the pg dose level (FIG. 23B).

Analysis on day 35 of CD8⁺ and CD4⁺ T cells in lung tissue of aged mice showed a similar trend, with AMP-CpG immunized animals producing high frequencies of cytokine producing CD8⁺ and CD4⁺ T cells. Specifically, AMP-CpG immunization elicited Th1 cytokine production in approximately 60% of lung resident CD8⁺ T cells, approximately 7-fold and >120-fold higher than soluble CpG and alum immunization (FIG. 23C). As observed in prior studies, the elicited T cells were highly polyfunctional with more than half of the induced cells exhibiting simultaneous production of IFNγ and TNFα. Unlike the responses in peripheral blood, lung-resident cytokine producing CD8⁺ T cell frequencies did not decline in aged mice following AMP-CpG immunization relative to responses in young healthy animals and were maintained at statistically comparable levels in the 5 μg and 1 μg coronavirus spike protein dosed groups (FIG. 23D). Lung resident CD4⁺ T cell responses exhibited a similar pattern with AMP-CpG inducing higher frequencies of Th1 cytokine producing cells (approximately 10% of CD4⁺ T cells) compared to soluble CpG (approximately 0.6% of CD4⁺ T cells) and alum (<0.5% of CD4⁺ T cells) (FIG. 23E). Again, these response levels were comparable to those observed in young healthy mice showing that AMP-CpG immunization can raise comparable lung-resident T cell responses in young and aged mice. Finally, the lung-resident CD4⁺ T cell responses were maintained at comparable levels among all coronavirus spike protein dose levels tested, and AMP-CpG immunization at the lowest concentration of coronavirus spike protein (1 μg) outperformed both soluble CpG and alum at a 10-fold higher antigen dose (10 μg) (FIG. 23F).

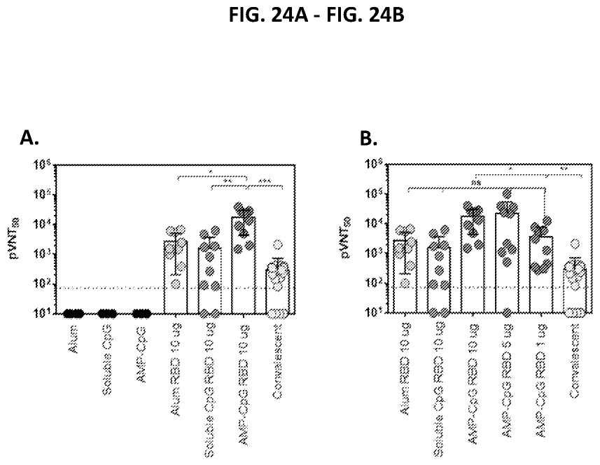

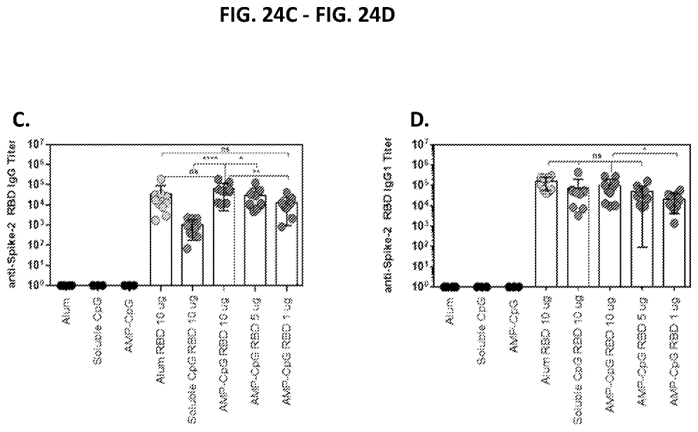

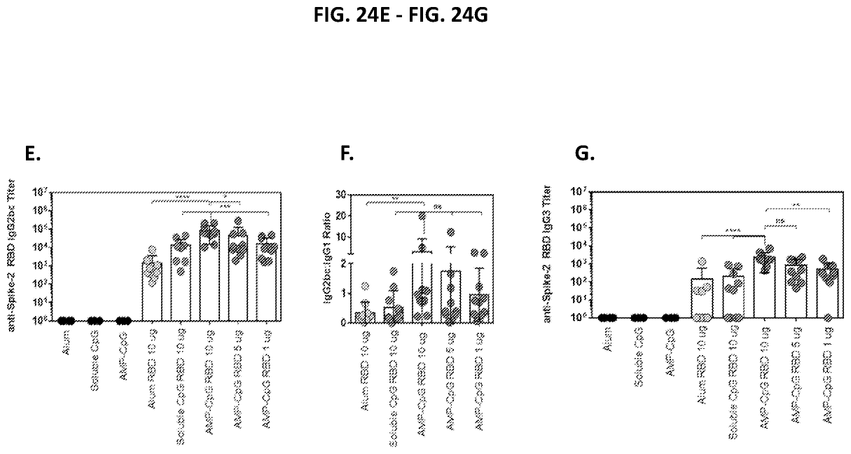

Coronavirus spike protein-specific antibody responses were evaluated on day 35 after repeat dose immunization with comparator vaccines in aged mice. Pseudovirus neutralization showed that AMP-CpG immunization at the 10 μg antigen dose level elicited enhanced neutralizing titers, at least 5-fold greater than those observed for soluble CpG and alum comparators, and >50-fold greater than observed in human convalescent sera/plasma (FIG. 24A). Reduced doses of coronavirus spike protein with AMP-CpG gave lower neutralizing titers which were comparable to soluble CpG and alum (FIG. 24B). Of particular interest was the equivalency of titers from animals immunized with 10 μg coronavirus spike protein with soluble CpG or alum relative to those receiving the lower 1 μg coronavirus spike protein dose with AMP-CpG. Assessment of total IgG showed AMP-CpG and alum produced comparable coronavirus spike protein-specific IgG titers, both in excess of that generated in soluble CpG immunized animals (FIG. 24C). Although a significant decline was observed in IgG titer with decreasing coronavirus spike protein dose in AMP-CpG immunized animals, there was no statistical difference between AMP-CpG and alum given with 10 μg coronavirus spike protein. Isotype analysis yielded similar observations to those made in young healthy mice, with AMP-CpG driving more Th1, IgG2bc-dominant responses compared with soluble CpG or alum, which yielded more balanced or Th1, IgG1-biased profiles (FIG. 24D-FIG. 24G). No significant difference was observed among AMP-CpG immunized animals at the varying dose levels of coronavirus spike protein (FIG. 24F), although the strength of Th1-bias observed for AMP-CpG immunized mice was reduced in aged mice relative to young healthy mice. As previously observed in young healthy mice, IgG3 titers were enhanced in AMP-CpG immunized animals compared with soluble CpG or alum (FIG. 24G). Together, these results support AMP-CpG being able to elicit potent and functional coronavirus spike protein-specific humoral immunity in aged mice beyond what was observed for soluble CpG or alum vaccine comparators while producing an optimal Th1-biased isotype profile and enabling at least 10-fold dose sparing of antigen.

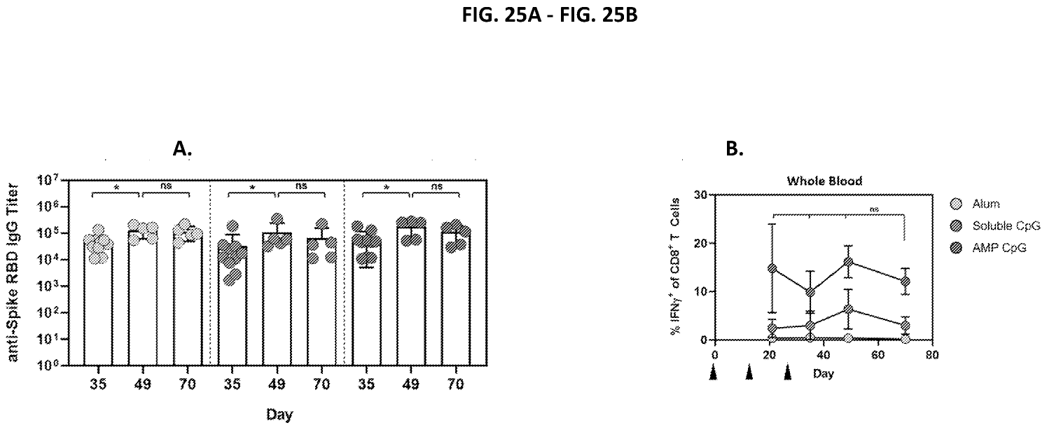

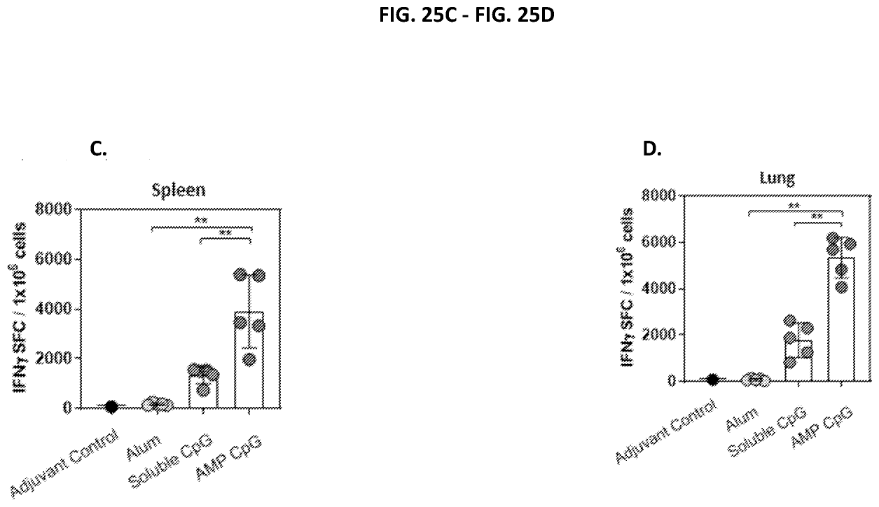

Vaccination with AMP-CpG in aged mice enables durable Spike RBD-specific T cells in blood, spleen, and lung tissue. 37 week old C57Bl/6 mice (n=5-10 per group) were immunized on day 0, 14, and 28 with 10 ug Spike RBD protein admixed with 100 ug Alum or 1 nmol soluble-, or AMP-CpG. Adjuvant control animals were dosed with AMP-CpG adjuvant alone. Humoral responses specific to Spike RBD were assessed in serum from immunized animals by ELISA on day 35, 49, and 70. Shown are endpoint titers determined for IgG (FIG. 25A). T cell responses were analyzed on day 21, 35, 49, and 70. Cells were collected from peripheral blood on day 21, 35, 49, and 70 (FIG. 25B) and were restimulated with overlapping Spike RBD peptides and assayed for intracellular cytokine production to detect antigen-specific T cell responses. Shown are frequencies of IFNγ-positive cells among peripheral blood CD8⁺ T cells (FIG. 25A), and cells were collected from spleen (FIG. 25C) and lungs (FIG. 25D) and were restimulated with overlapping Spike RBD peptides and assayed for IFNγ production by ELISPOT assay. Shown are the frequency of IFNγ spot forming cells (SFC) per 1×10⁶ cells (n=5 mice per group). Values depicted are mean±standard deviation. * P<0.05; ** P<0.01; *** P<0.001; **** P<0.0001 by two-sided Mann-Whitney test applied to cytokine+ T cell frequencies.

These results show that, in aged mice, AMP-CpG induces high frequency T cell responses that persist for months after dosing.

### Example 5: Inducing an Immune Response Using a Full-Length Spike Protein Antigen and a Nucleocapsid Protein Antigen

The SARS-CoV-2 spike protein has a molecular weight of approximately 138 kDa and the SARS-CoV-2 nucleocapsid protein has a molecular weight of approximately 50 kDa. Based on these sizes, both the spike protein and the nucleocapsid protein are predicted to be suitable for lymph node targeting.

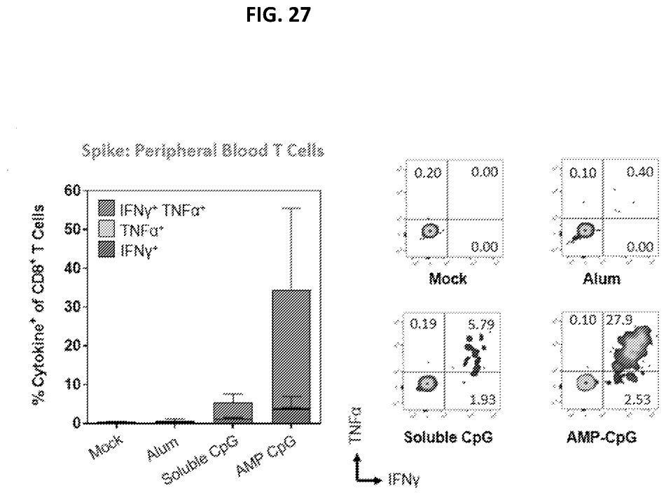

The following nucleocapsid protein construct was used to generate the data shown in FIG. 27-FIG. 33:

(SEQ ID NO: 63)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWWVATEGALNTPKDHIGTR
NPANNAAIVLQLPQGTTLPKGFYAEGSRGGSQASSRSSSRSRNSSRNSTP
GSSRGTSPARMAGNGGDAALALLLLDRLNQLESKMSGKGQQQQGQTVTKK
SAAEASKKPRQKRTATKAYNVTQAFGRRGPEQTQGNFGDQELIRQGTDYK
HWPQIAQFAPSASAFFGMSRIGMEVTPSGTWLTYTGAIKLDDKDPNFKDQ
VILLNKHIDAYKTFPPTEPKKDKKKKADETQALPQRQKKQQTVTLLPAAD
LDDFSKQLQQSMSSADSTQAENLYFQGHHHHHH.

This protein is available from ACROBiosystems under product number NUN-05227. It includes a cleavage site for a tobacco etch virus (TEV) protease (ENLYFQG; SEQ ID NO:64) between the nucleocapsid protein sequence and the six-histidine tag (HHHHHH; SEQ ID NO:65).

The following full-length spike protein construct was used to generate the data shown in FIG. 27-FIG. 33:

(SEQ ID NO: 66)
VNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRD
LPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQT
SNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIP
IGAGICASYQTQTNSPRAAASVASQSIIAYTMSLGAENSVAYSNNSIAIP
TNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKR
SFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNV
LYENQKLIANQFNSAIGKIQDSLSSTASALGKLQDVVNQNAQALNTLVKQ
LSSNFGAISSVLNDILSRLDKVEAEVQIDRLITGRLQSLQTYVTQQLIRA
AEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPGG
GSGGGSHHHHHHHHHH.

This protein is available from ACROBiosystems under product number SPN-052H2. A ten-histidine tag (HHHHHHHHHH; SEQ ID NO:67) is linked to the spike protein sequence with a GGGSGGGS (SEQ ID NO:62) linker. The spike protein has the following mutations to stabilize the trimer: R683A, R685A.

As shown in FIG. 27, AMP-CpG induces a potent polyfunctional CD8 T cell response targeting SARS CoV-2 spike protein. A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0%), alum (0%), soluble CpG (5%), and AMP-CpG (34%).

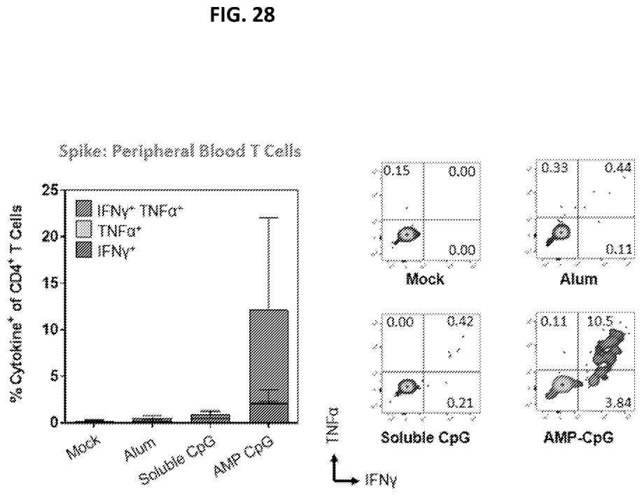

As shown in FIG. 28, AMP-CpG also induces a potent polyfunctional CD4 T cell response targeting SARS CoV-2 spike protein. A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0.2%), alum (0.5%), soluble CpG (0.5%), and AMP-CpG (12%).

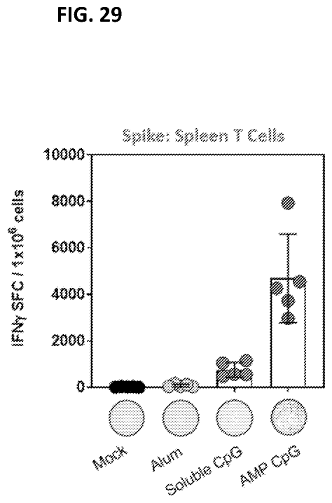

Restimulating mice (C57BL/6J mice; n=10 per group) that had received 10 μg of a full-length coronavirus spike protein construct (SEQ ID NO: 66) in combination with 10 μg of a coronavirus nucleocapsid protein construct (SEQ ID NO:63) and (1) 100 μg alum, (2) 6 μg soluble CpG, or (3) 6 μg AMP-CpG with overlapping coronavirus spike peptides resulted in a potent T cell response against SARS CoV-2 spike protein (FIG. 29).

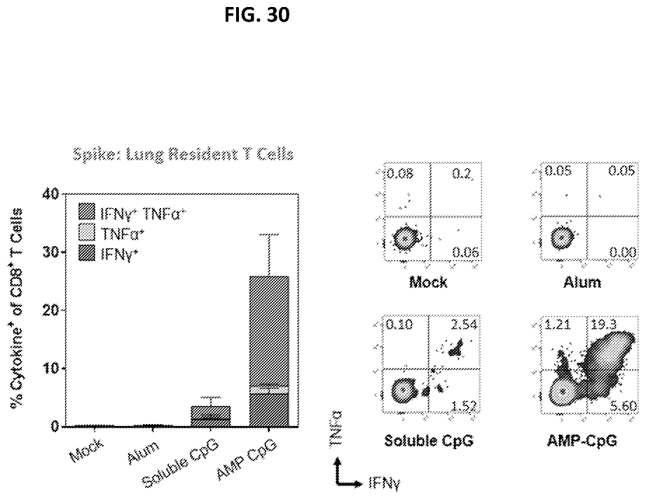

AMP-CpG induces a potent lung-resident polyfunctional CD8⁺ T cell response targeting SARS CoV-2 spike protein (FIG. 30). A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0%), alum (0%), soluble CpG (3%), and AMP-CpG (26%).

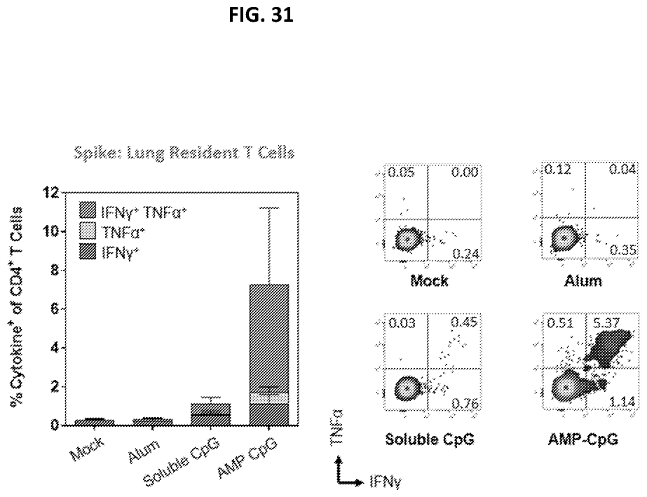

AMP-CpG also induces a potent lung-resident polyfunctional CD4⁺ T cell response targeting SARS CoV-2 spike protein (FIG. 31). A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered. The percent cytokine positive cells observed were: mock (0.2%), alum (0.2%), soluble CpG (1%), and AMP-CpG (7%).

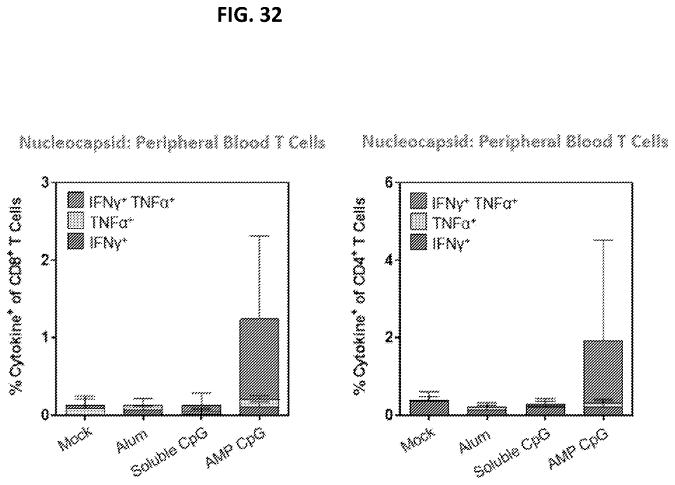

AMP-CpG induces a potent peripheral blood polyfunctional CD8⁺ and CD4⁺ T cell response targeting SARS CoV-2 nucleocapsid protein (FIG. 32). A mock vaccine, or a vaccine containing 10 μg coronavirus spike protein, 10 μg coronavirus nucleocapsid protein and (1) 100 μg alum, (2) 6 μg soluble CpG, or (3) 6 μg AMP-CpG was administered.

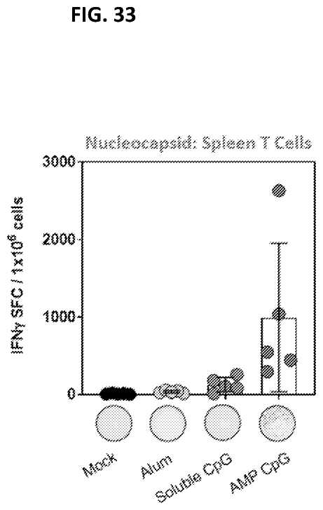

Restimulating mice (C57BL/6J mice; n=10 per group) that had received 10 μg of a full-length coronavirus spike protein construct (SEQ ID NO: 66) in combination with 10 μg of a coronavirus nucleocapsid protein construct (SEQ ID NO:63) and (1) 100 μg alum, (2) 6 μg soluble CpG, or (3) 6 μg AMP-CpG with overlapping coronavirus nucleocapsid peptides induced a potent T cell response targeting SARS CoV-2 nucleocapsid protein (FIG. 33).

### Example 6: Inducing an Immune Response in Non-Human Primates

A study was initiated in non-human primates (NHP) to test spike RBD and AMP-CpG in a vaccine. Use of RBD+Alum in the vaccine was compared to RBD+AMP-CpG. An initial dose of 500 μg of AMP-CpG was tested in a two-dose schedule (week 0 and week 4) immunized subcutaneously. Assessments included weekly clinical examination post each dose, CBC (complete blood count) panel, and collection of blood and sera for immunogenicity. In these tests, AMP-CpG did not induce an antibody or T-cell response to BioE spike RBD. As no response was seen to AMP-CpG after 2 doses, the same animals were immunized with a new vaccine formulation. Here 3,000 μg of AMP-CpG were used and 140 μg Genscript RBD were used. (A different lot and higher concentration of AMP-CpG and a new source and higher concentration of RBD.) The comparison group remained the same (1.5 mg Alum+70 μg BioE RBD).

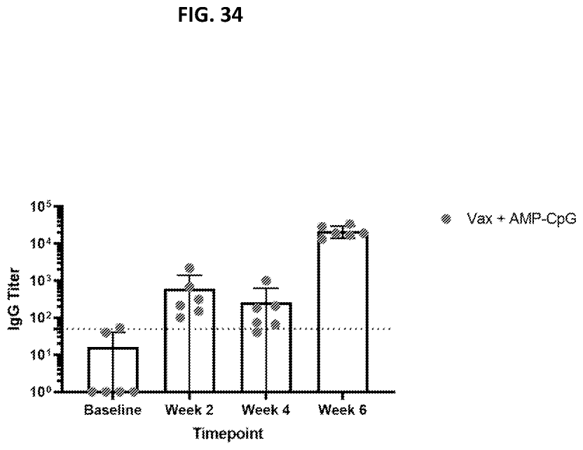

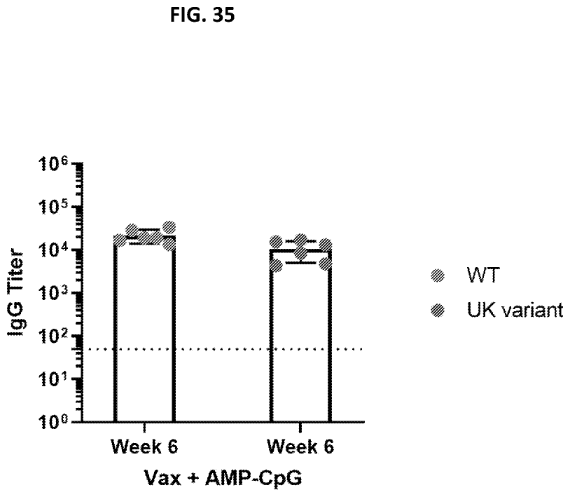

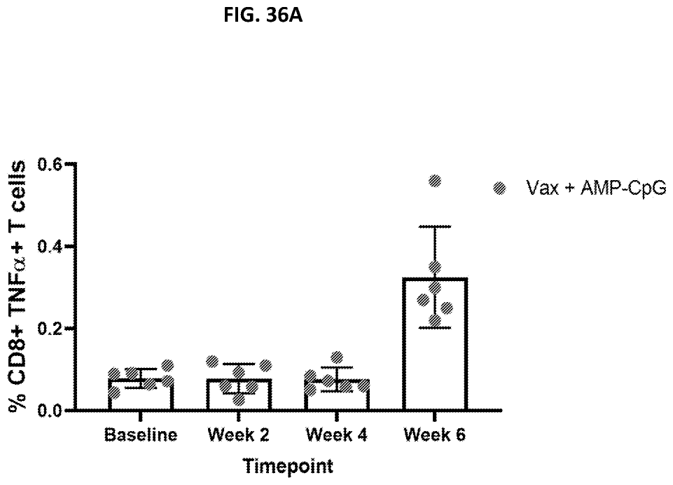

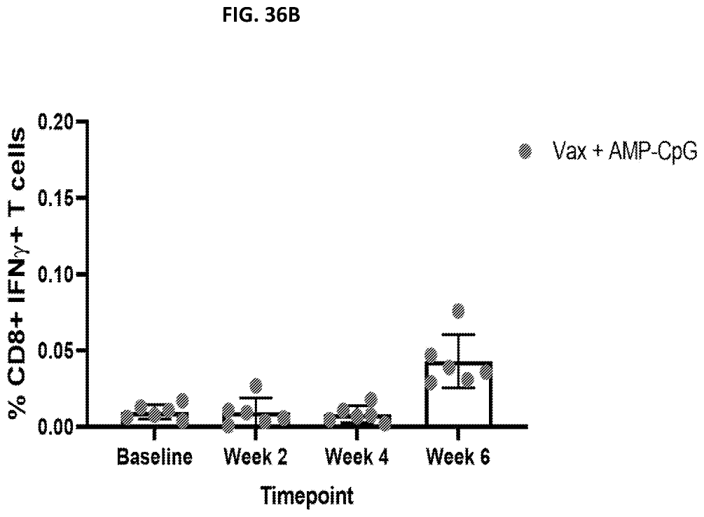

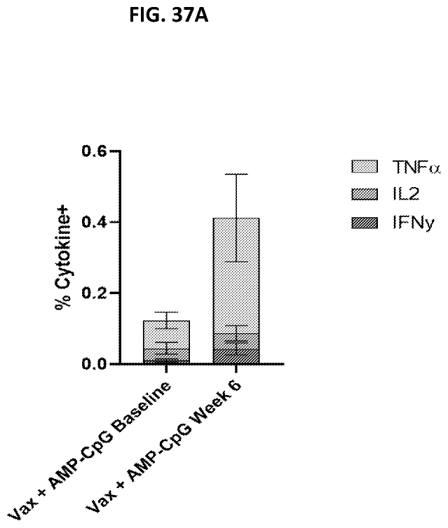

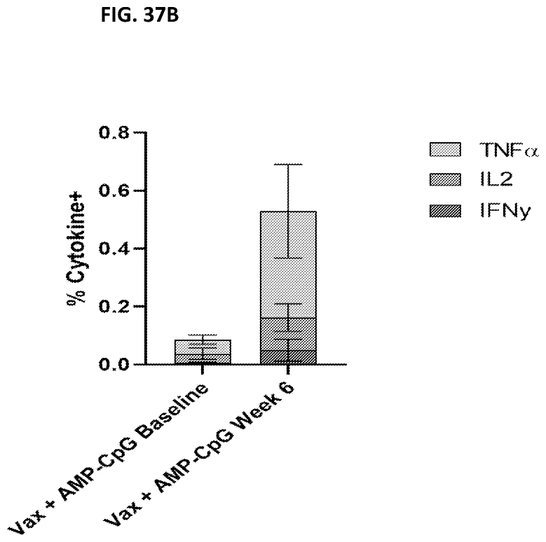

The reformulated AMP-CpG vaccine induced a robust antibody response to Genscript RBD (FIG. 34.) The reformulated AMP-CpG vaccine also induces IgG antibodies to the UK SARS-CoV-2 variant having the N501Y mutation (SEQ ID NO:69) (FIG. 35). Further, the reformulated AMP-CPG vaccine induces CD8⁺ T-cell responses to spike RBD (FIG. 36A and FIG. 36B), and CD4⁺ and CD8⁺ T-cell responses to spike RBD (FIG. 37A and FIG. 37B).

No adverse safety signals (temperature, reactogenicity, chemistry, and hematology) were observed for the reformulated RBD and AMP-CpG vaccine.

### Example 7: Inducing an Immune Response to B.1.351 Variant

The B.1.351, or South Africa, variant of COVID is a strain of SARS-CoV2. A study was initiated in mice to determine the immunogenicity of the spike RBD and AMP-CpG in a vaccine if the antigen is changed to the B.1.351 RBD variant or used in conjunction with the WT RBD antigen. The cross-reactivity of the immune response towards the different variants was also determined for the reformulated AMP-CPG dual RBG and B.1.351 vaccine and the reformulated AMP-CPG B.1.351 vaccine.

Control, WT RBG, B.1.351 RGB, and dual WT RBG and B.1.351 stock solutions were prepared. Control adjuvant stock solutions were resuspended in limulus amebocyte lysate (LAL) water. Final injections were diluted 1× Phosphate-buffered saline (PBS). SARS-CoV2 Spike S1 RBD protein stock solutions comprising the WT and B.1.351 antigens were dissolved in PBS at a concentration of 0.88 and 0.95 mg/ml, respectively, having 5 μg per 100 μl injection. Final injections were diluted with 1×PBS. A dual WT and B.1.351 SARS-CoV2 Spike S1 RBD protein stock solution was also prepared with 5 μg of each antigen per 100 μl injection.

TABLE 5
Experimental Design

| | | | | Dosing and Sample Collection | | | |
|---|---|---|---|---|---|---|---|
| | | Vaccine Components | | Day 1 | Day 13 | Day 14 | Day 21 |
| Group | Treatment Name | Antigen (5 ug) | Adjuvant (1 nmol) | Dose 1 | Read-out | Dose 2 | Read-out |
| 1 | AMP Vax (B.1.351) | B.1.351 RBD | DSPE-PEG-CpG7909 | x | PBMCs | x | PBMC/ |
| 2 | AMP Dual Vax | B.1.351 RBD + | DSPE-PEG-CpG7909 | x | ICS | x | Lung |
| | | WT RBD | | | Serum Ab | | ICS |
| 3 | AMP Vax (WT) | WT RBD | DSPE-PEG-CpG7909 | x | ELISA | x | Serum |
| 4 | Sol Vax (B.1.351) | — | CpG7909 | x | | x | Spleen |
| 4 | Adj Ctrl | — | DSPE-PEG-CpG7909 | x | | x | ELISpot |

5 groups of 5 C57BL/6J mice each were used. Immunizations were administered subcutaneously (SC) into the tail base of female B6 mice, bilaterally, 50 μl per side. Booster doses were given at roughly 2-week intervals. SC injections may aid in delivering the vaccine into the lymph nodes via natural lymph drainage. Bi-weekly injections may aid in optimal response generation in mice based on previous mouse studies.

TABLE 6
Vaccine Components

| Vaccine | | | |
|---|---|---|---|
| Components | Sequence or Cat# | Source | Lot # |
| SARS-COV2 | Z03483 | GenScript | P50142007 |
| RBD, His | | | |
| (WT) | | | |
| SARS-COV2 | Z03537 | GenScript | B2101019 |
| RBD, His | | | |
| (B.1.351) | | | |
| aCpG 7909 | 5′-(Diacyl lipid) | Avecia | S18-079- |
| | tcg tcg ttt tgt | | S3-B1 |
| | cgt ttt gtc gtt-3′ | | |
| | (SEQ ID NO: 1) | | |

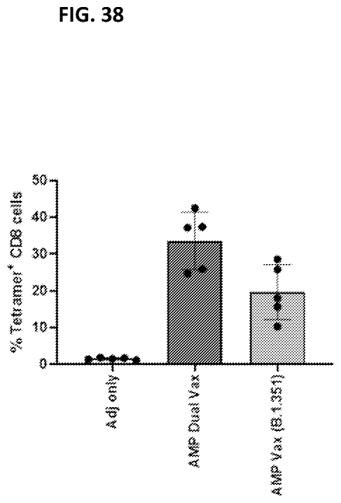

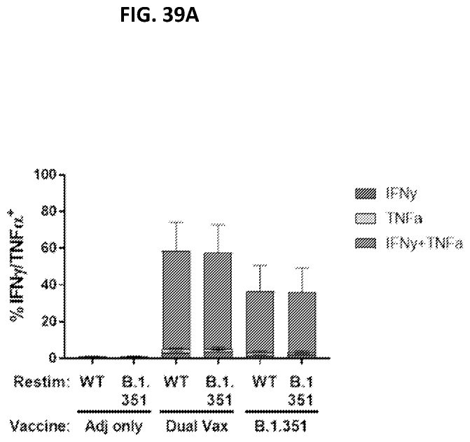

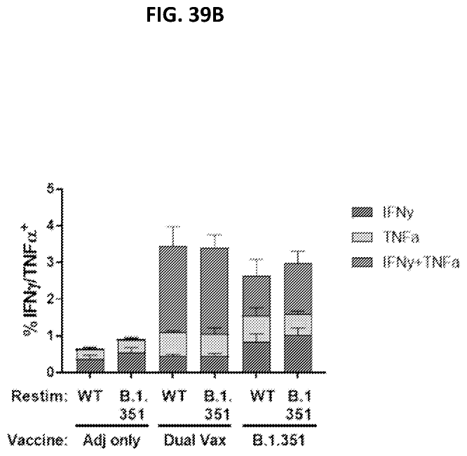

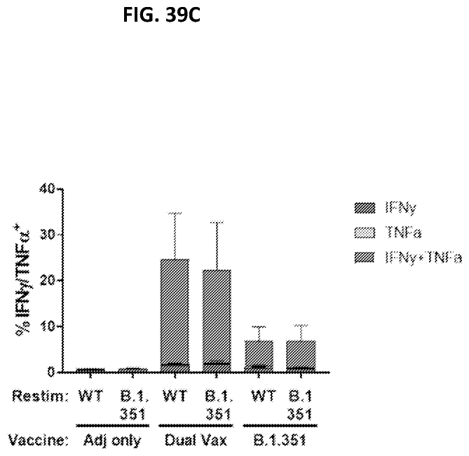

Tetramer analysis was conducted, and results are shown in FIG. 38. Intracellular Stain (ICS) Assay for TNFα and IFNγ was performed on PBMCs 7 days after dosing. ICS was also performed on lung samples 7 days post dose 2. Cells were surface stained for CD4, CD8, and CD3. See Table 7 for antibody information. ICS samples were activated overnight (in the presence of Brefeldin A and Monensin) with 1 mg per well of SARS-CoV-2 Spike Glycoprotein Peptide Pool Mix (315 peptides each at 1 mg per well). Results are shown in FIG. 39A, FIG. 39B, and FIG. 39C for CD8⁺ lung cells, CD4⁺ lung cells, and CD8⁺ lung cells respectively following dose 2. See Table 8 for peptide information.

TABLE 7
Antibodies Used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 8
Re-Stimulation Peptides

| Re-stimulation | | | |
|---|---|---|---|
| Peptides | Sequence | Source | Lot # |
| SARS-CoV-2 Spike | 315 15mers spanning | GenScript | custom |
| Glycoprotein Peptide | Spike Protein Sequence, | | |
| Pool Mix | overlap 11aa | | |

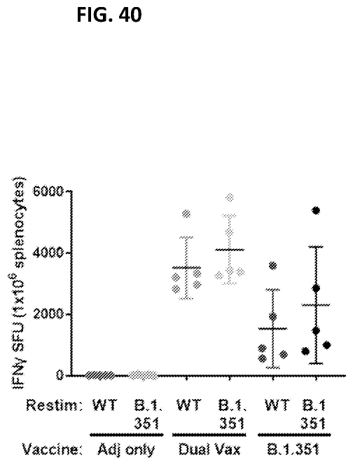

ELISpot analysis for IFNγ was performed on splenocytes after dose 3 administration. Splenocytes (0.2×106 cells/well) were activated with 1 μg per well PepMix. See Table 8 for peptide information. IFNγ plates were stimulated overnight. Results are shown in FIG. 40.

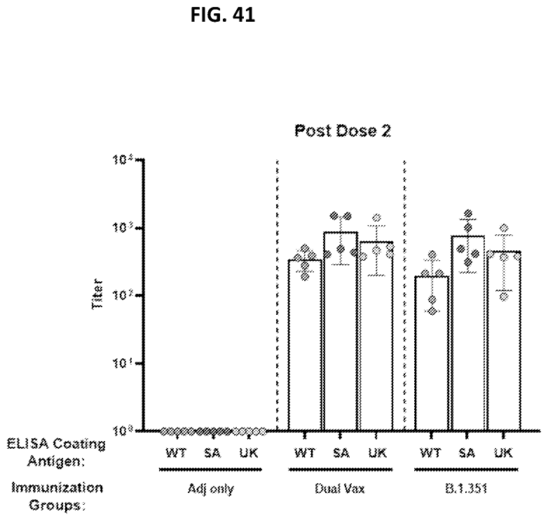

SARS-CoV2 specific serum ELISA (enzyme-linked immunosorbent assay) was performed on mouse serum 7 days after each dose to detect any RBD-specific antibody response. Whole blood was centrifuged using Ser-gel tubes (NC9436363, Fisher Scientific). Serum was either used fresh or stored at −80° C. until used. 96-well plates were coated with 200 ng/100 μl (2 μg/ml) of CoV2 RBD protein (WT, B.1.351 and B.1.1.7) overnight at 4° C. Then plates were pre-blocked with 2% BSA for 2 h at RT. Mouse serum was diluted 1:20 and serially diluted (1:5 to 8 concentrations) in a dummy plate. ELISA plates were washed once with ELISA washing buffer (BioLegend 4211601). Samples were transferred to the ELISA plate and incubated for 2 h at RT. Plates were washed 4 times with washing buffer. For serum antibody detection the secondary HRP-conjugated antibodies in Table 9 were used at 1:2000 in PBS+ and incubated for 1 h at room temperature. Plates were washed 4 times with washing buffer. The reaction was visualized by addition of substrate 3,3′,5,5′-Tetramethylbenzidine (TMB) for 10 min at RT and stopped by H2SO4 (1 N). The absorbance at 450 nm was measured by an ELISA plate reader. Results are shown in FIG. 41.

TABLE 9
Secondary HRP-Conjugated Antibodies

| Antigen | Source | Product # | Lot # |
|---|---|---|---|
| IgG | Jackson Immunoresearch | 315-035-046 | 147406 |

The reformulated AMP-CPG dual WT RBD and B.1.351 RBD vaccine and the reformulated AMP-CPG B.1.351 RBD vaccine elicits similar immunological responses to the vaccine that uses solely the WT RBD antigen seen in previous experiments. The T-cell as well as antibody responses are equally cross-reactive against all tested variants of SARS-CoV2 RBD.

### Example 8: Inducing an Immune Response in Human Subjects

According to the methods disclosed herein, a subject, such as a human subject, can be administered a CpG amphiphile and a coronavirus antigen (e.g., a coronavirus spike protein or a peptide thereof, e.g., a RBD peptide, and/or a coronavirus nucleocapsid protein or a peptide thereof, or a nucleic acid sequence encoding the same) to induce an immune response in the subject. To this end, the patient is administered a CpG amphiphile and the coronavirus antigen (e.g., a coronavirus spike protein or a peptide thereof, e.g., a RBD peptide, and/or a coronavirus nucleocapsid protein or a peptide thereof, or a nucleic acid sequence encoding the same). The CpG amphiphile or a pharmaceutical composition thereof is administered to the subject subcutaneously in the form of a vaccine. The CpG amphiphile or a pharmaceutical composition thereof may also be administered intranasally, intratracheally, or by inhalation during mechanical ventilation. The subject is also administered a coronavirus antigen (e.g., a coronavirus spike protein or a peptide thereof, e.g., a RBD peptide, and/or a coronavirus nucleocapsid protein or a peptide thereof, or a nucleic acid sequence encoding the same) or a pharmaceutical composition thereof subcutaneously in the form of a vaccine. The coronavirus antigen or a pharmaceutical composition thereof may also be administered intranasally, intratracheally, or by inhalation during mechanical ventilation. Both the CpG amphiphile and the coronavirus antigen (e.g., a coronavirus spike protein or a peptide thereof, e.g., a RBD peptide, and/or a coronavirus nucleocapsid protein or a peptide thereof, or a nucleic acid sequence encoding the same) may be administered bilaterally on the inner thigh. The CpG amphiphile and the (e.g., a coronavirus spike protein or a peptide thereof, e.g., a RBD peptide, and/or a coronavirus nucleocapsid protein or a peptide thereof, or a nucleic acid sequence encoding the same) may be administered separately or concurrently to the subject. The subject may receive a dosage of the CpG amphiphile and the (e.g., a coronavirus spike protein or a peptide thereof, e.g., a RBD peptide, and/or a coronavirus nucleocapsid protein or a peptide thereof, or a nucleic acid sequence encoding the same) at week 0, week 4, and week 10 or at week 0 and week 4. The subject may receive a dosage of about 0.1 mg to 20.0 mg. In particular, the dosage administered may be in the range of about 0.1 mg to 1.0 mg, of about 0.5 mg to 3.0 mg, of about 1.0 mg to 5.0 mg, of about 2.0 to 5.0 mg, of about 3.0 to 5.0 mg, of about 3.0 mg to 10.0 mg, of about 4.0 mg to 12.0 mg, of about 5.0 mg to 15.0 mg, or of about 5.0 to 20.0 mg. The particular dosage administered to the subject may be about 0.1 mg, 0.2 mg, 0.3 mg, 0.4 mg, 0.5 mg, 1.0 mg, 2.0 mg, 3.0 mg, 4.0 mg, 5.0 mg, 6.0 mg, 7.0 mg, 8.0 mg, 9.0 mg, 10.0 mg, 11.0 mg, 12.0 mg, 13.0 mg, 14.0 mg, 15.0 mg, 16.0 mg, 17.0 mg, 18.0 mg, 19.0 mg, or 20.0 mg of the CpG amphiphile. The subject also may receive a dosage in a range between any two of these particular dosages of the CpG amphiphile. The subject may receive a dosage of about 10 μg to about 1.0 mg of the coronavirus antigen. In particular, the subject may receive a dosage of about 40 μg to 60 μg, of about 50 μg to 70 μg, of about 50 μg to 150 μg, of about 70 μg to 150 μg, of about 100 μg to 150 μg, of about 100 μg to 200 μg, of about 140 μg to 250 μg, of about 200 μg to 300 μg, of about 250 μg to 500 μg, of about 300 μg to 600 μg, or of about 500 μg to 1.0 mg of the corona virus antigen. In particular, the dosage administered to the subject may be about 10 μg, 20 μg, 30 μg, 40 μg, 50 μg, 60, pg, 70 μg, 80 μg, 90 μg, 100 μg, 110 μg, 120 μg, 130 μg, 140 μg, 150 μg, 200 μg, 250 μg, 300 μg, 400 μg, 500 μg, 600 μg, 700 μg, 800 μg, 900 μg, or 1.0 mg of the coronavirus antigen (e.g., spike protein or spike protein RBD). The subject also may receive a dosage in a range between any two of these particular dosages of the coronavirus antigen.

### OTHER EMBODIMENTS

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All publications, patents, and patent applications are herein incorporated by reference in their entirety to the same extent as if each individual publication, patent, or patent application was specifically and individually indicated to be incorporated by reference in its entirety.

What is claimed is:

1. A method of inducing an immune response against a coronavirus antigen in a subject, the method comprising administering (1) a CpG-amphiphile and (2) a coronavirus antigen or a nucleic acid sequence encoding the coronavirus antigen to the subject.

2. The method of claim 1, wherein the coronavirus antigen is a coronavirus spike protein or a peptide thereof or a nucleic acid sequence encoding the coronavirus spike protein or peptide.

3. The method of claim 1 or 2, wherein the CpG-amphiphile comprises a CpG sequence bonded to a lipid.

4. The method of claim 1 or 2, the CpG-amphiphile comprises a CpG sequence linked to a lipid by a linker.

5. The method of claim 4, wherein the linker comprises a polymer, a string of amino acids, a string of nucleic acids, a polysaccharide, or a combination thereof.

6. The method of claim 5, wherein the linker comprises a string of nucleic acids.

7. The method of claim 6, wherein the string of nucleic acids comprises between 1 and 50 nucleic acid residues.

8. The method of claim 7, wherein the string of nucleic acids comprises between 5 and 30 nucleic acid residues.

9. The method of any one of claims 5-8, wherein the string of nucleic acids comprises “N” guanines, wherein N is 1-10.

10. The method of claim 5, wherein the linker comprises consecutive polyethylene glycol units.

11. The method of claim 10, wherein the linker comprises “N” consecutive polyethylene glycol units, wherein N is between 20 and 80.

12. The method of claim 11, wherein the linker comprises “N” consecutive polyethylene glycol units, wherein N is between 30 and 70.

13. The method of claim 12, wherein the linker comprises “N” consecutive polyethylene glycol units, wherein N is between 40 and 60.

14. The method of claim 13, wherein the linker comprises “N” consecutive polyethylene glycol units, wherein N is between 45 and 55.

15. The method of claim 14, wherein the linker comprises 48 consecutive polyethylene glycol units.

16. The method of any one of claims 1-15, wherein the lipid is a diacyl lipid.

17. The method of claim 16, wherein the diacyl lipid has the following structure: 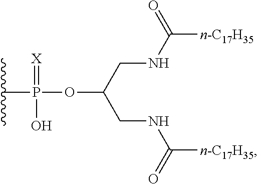
or a salt thereof,
wherein X is O or S.

18. The method of any one of claims 1-17 wherein the CpG sequence comprises the nucleotide sequence 5′-TCGTCGTTTTGTCGTTTTGTCGTT-3′ (SEQ ID NO:1).

19. The method of any one of claims 1-17, wherein the CpG sequence comprises the nucleotide sequence of 5′-TCCATGACGTTCCTGACGTT-3′ (SEQ ID NO: 2).

20. The method claim 18 or claim 19, wherein all internucleoside groups connecting the nucleosides in the CpG sequence are phosphorothioates.

21. The method of any one of claims 1-20, wherein the coronavirus spike protein or peptide thereof is a SARS-CoV-2 spike protein or peptide thereof.

22. The method of any one of claims 1-21, wherein the peptide of the coronavirus spike protein is a receptor binding domain that specifically binds angiotensin-converting enzyme 2 (ACE2).

23. The method of any one of claims 1-22, wherein the peptide of the coronavirus spike protein comprises a polypeptide sequence having at least 90% sequence identity to:
(SEQ ID NO: 3)
RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRD
IADTTDAVRDPQTLEILDITPCS.

24. The method of claim 23, wherein the peptide of the coronavirus spike protein comprises the polypeptide sequence of:
(SEQ ID NO: 3)
RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRD
IADTTDAVRDPQTLEILDITPCS.

25. The method of any one of claims 1 and 3-21, wherein the coronavirus antigen is a coronavirus nucleocapsid protein or a peptide thereof.

26. The method of claim 25, wherein the coronavirus nucleocapsid protein antigen comprises a polypeptide sequence having at least 90% sequence identity to:
(SEQ ID NO: 68)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIVATEGALNTPKDHIGTRNP
ANNAAIVLQLPQGTTLPKGFYAEGSRGGSQASSRSSSRSRNSSRNSTPGS
SRGTSPARMAGNGGDAALALLLLDRLNQLESKMSGKGQQQQGQTVTKKSA
AEASKKPRQKRTATKAYNVTQAFGRRGPEQTQGNFGDQELIRQGTDYKHW
PQIAQFAPSASAFFGMSRIGMEVTPSGTWLTYTGAIKLDDKDPNFKDQVI
LLNKHIDAYKTFPPTEPKKDKKKKADETQALPQRQKKQQTVTLLPAADLD
DFSKQLQQSMSSADSTQA.

27. The method of claim 25, wherein the coronavirus nucleocapsid protein antigen comprises the polypeptide sequence of:
(SEQ ID NO: 63)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIVATEGALNTPKDHIGTRNP
ANNAAIVLQLPQGTTLPKGFYAEGSRGGSQASSRSSSRSRNSSRNSTPGS
SRGTSPARMAGNGGDAALALLLLDRLNQLESKMSGKGQQQQGQTVTKKSA
AEASKKPRQKRTATKAYNVTQAFGRRGPEQTQGNFGDQELIRQGTDYKHW
PQIAQFAPSASAFFGMSRIGMEVTPSGTWLTYTGAIKLDDKDPNFKDQVI
LLNKHIDAYKTFPPTEPKKDKKKKADETQALPQRQKKQQTVTLLPAADLD
DFSKQLQQSMSSADSTQAENLYFQGHHHHHH.

28. The method of any one of claims 1-26, wherein the coronavirus antigen comprises one or more tags.

29. The method of claim 28, wherein the tag is an Avi tag.

30. The method of claim 28, wherein the tag is a histidine tag.

31. The method of any one of claims 28-30, wherein the coronavirus antigen comprises an Avi tag and a histidine tag.

32. The method of any one of claims 28-31, wherein the coronavirus antigen comprises a linker between the polypeptide sequence and the one or more tags.

33. The method of any one of claims 1-24 and 28-31, wherein the coronavirus spike protein is administered.

34. The method of claim 33, wherein a trimer of the coronavirus spike protein is administered.

35. The method of claim 34, wherein the trimer is a trimer of a protein construct comprising the sequence:
(SEQ ID NO: 66)
VNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRD
LPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQT
SNFRVQPTESIVRFPNITNLCPFGEVFNATRFASVYAWNRKRISNCVADY
SVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIP
IGAGICASYQTQTNSPRAAASVASQSIIAYTMSLGAENSVAYSNNSIAIP
TNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKR
SFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNV
LYENQKLIANQFNSAIGKIQDSLSSTASALGKLQDVVNQNAQALNTLVKQ
LSSNFGAISSVLNDILSRLDKVEAEVQIDRLITGRLQSLQTYVTQQLIRA
AEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPGG
GSGGGSHHHHHHHHHH.

36. The method of claim one of claims 1-35, wherein a coronavirus spike protein, or a peptide thereof, and a coronavirus nucleocapsid protein, or a peptide thereof, are administered.

37. The method of claim 36, wherein a trimer of a coronavirus spike protein construct comprising the sequence:
VNLTTRTQLPPAYTNSFTRGVYYPDKVFRSSVLHSTQDLFLPFFSNVTWFHAIHVSGTNGTKRFDNPVLP FNDGVYFASTEKSNIIRGWIFGTTLDSKTQSLLIVNNATNVVIKVCEFQFCNDPFLGVYYHKNNKSWMESE FRVYSSANNCTFEYVSQPFLMDLEGKQGNFKNLREFVFKNIDGYFKIYSKHTPINLVRDLPQGFSALEPLV DLPIGINITRFQTLLALHRSYLTPGDSSSGWTAGAAAYYVGYLQPRTFLLKYNENGTITDAVDCALDPLSET KCTLKSFTVEKGIYQTSNFRVQPTESIVRFPNITNLCPFGEVFNATRFASVYAWNRKRISNCVADYSVLYN SASFSTFKCYGVSPTKLNDLCFTNVYADSFVIRGDEVRQIAPGQTGKIADYNYKLPDDFTGCVIAWNSNN LDSKVGGNYNYLYRLFRKSNLKPFERDISTEIYQAGSTPCNGVEGFNCYFPLQSYGFQPTNGVGYQPYR VVVLSFELLHAPATVCGPKKSTNLVKNKCVNFNFNGLTGTGVLTESNKKFLPFQQFGRDIADTTDAVRDP QTLEILDITPCSFGGVSVITPGTNTSNQVAVLYQDVNCTEVPVAIHADQLTPTWRVYSTGSNVFQTRAGCL IGAEHVNNSYECDIPIGAGICASYQTQTNSPRAAASVASQSIIAYTMSLGAENSVAYSNNSIAIPTNFTISVT TEILPVSMTKTSVDCTMYICGDSTECSNLLLQYGSFCTQLNRALTGIAVEQDKNTQEVFAQVKQIYKTPPIK DFGGFNFSQILPDPSKPSKRSFIEDLLFNKVTLADAGFIKQYGDCLGDIAARDLICAQKFNGLTVLPPLLTD EMIAQYTSALLAGTITSGWTFGAGAALQIPFAMQMAYRFNGIGVTQNVLYENQKLIANQFNSAIGKIQDSL SSTASALGKLQDVVNQNAQALNTLVKQLSSNFGAISSVLNDILSRLDKVEAEVQIDRLITGRLQSLQTYVT QQLIRAAEIRASANLAATKMSECVLGQSKRVDFCGKGYHLMSFPQSAPHGWFLHVTYVPAQEKNFTTA PAICHDGKAHFPREGVFVSNGTHWFVTQRNFYEPQIITTDNTFVSGNCDWIGIVNNTVYDPLQPELDSFK EELDKYFKNHTSPDVDLGDISGINASVVNIQKEIDRLNEVAKNLNESLIDLQELGKYEQYIKWPGGGSGGG SHHHHHHHHHH (SEQ ID NO:66), and a coronavirus nucleocapsid protein construct having the polypeptide sequence of:
MSDNGPQNQRNAPRITFGGPSDSTGSNQNGERSGARSKQRRPQGLPNNTASWFTALTQHGKEDLKFP RGQGVPINTNSSPDDQIGYYRRATRRIRGGDGKMKDLSPRWYFYYLGTGPEAGLPYGANKDGIIWVATE GALNTPKDHIGTRNPANNAAIVLQLPQGTTLPKGFYAEGSRGGSQASSRSSSRSRNSSRNSTPGSSRGT SPARMAGNGGDAALALLLLDRLNQLESKMSGKGQQQQGQTVTKKSAAEASKKPRQKRTATKAYNVTQA FGRRGPEQTQGNFGDQELIRQGTDYKHWPQIAQFAPSASAFFGMSRIGMEVTPSGTWLTYTGAIKLDDK DPNFKDQVILLNKHIDAYKTFPPTEPKKDKKKKADETQALPQRQKKQQTVTLLPAADLDDFSKQLQQSMS SADSTQAENLYFQGHHHHHH (SEQ ID NO:63) are administered.

38. The method of any one of claims 1-25, 28-31, and 36, wherein an mRNA encoding the coronavirus antigen is administered.

39. The method of any one of claims 1-38, wherein the CpG-amphiphile and the coronavirus antigen or nucleic acid sequence encoding the same are administered concurrently.

40. The method of any one of claims 1-38, wherein the CpG-amphiphile and the coronavirus antigen, or nucleic acid sequence enclosing the same are administered sequentially.

41. The method of claim 40, wherein the CpG-amphiphile is administered first, followed by administering of the coronavirus antigen or nucleic acid sequence encoding the same.

42. The method of claim 40, wherein said the coronavirus antigen or nucleic acid sequence encoding the same is administered first, followed by administering of CpG-amphiphile.

43. The method of any one of claims 1-42, wherein the method comprises administering a second adjuvant to the subject.

44. The method of any one of claims 1-43, wherein the method comprises administering a coronavirus vaccine to the subject as a prime or a boost.

45. The method of any one of claims 1-44, wherein the CpG-amphiphile is administered subcutaneously, intranasally, intratracheally, or by inhalation during mechanical ventilation.

46. The method of claim 45, wherein the CpG-amphiphile is administered subcutaneously.

47. The method of any one of claims 1-46, wherein the coronavirus antigen is administered subcutaneously, intranasally, intratracheally, or by inhalation during mechanical ventilation.

48. The method of any one of claims 1-47, wherein the subject is a mammal.

49. The method of claim 48, wherein the subject is a human.

50. A pharmaceutical composition comprising a CpG-amphiphile and a coronavirus antigen, or a nucleic acid sequence encoding the coronavirus antigen, and a pharmaceutically acceptable carrier.

51. The pharmaceutical composition of claim 50, wherein the coronavirus antigen is a coronavirus spike protein or a peptide thereof.

52. The pharmaceutical composition of claim 50, wherein the coronavirus antigen is a coronavirus nucleocapsid protein or a peptide thereof.

53. The pharmaceutical composition of claim 50, wherein the coronavirus antigen comprises a coronavirus spike protein or a peptide thereof and a coronavirus nucleocapsid protein or a peptide thereof.

54. A kit comprising a CpG-amphiphile and a coronavirus antigen or a nucleic acid sequence encoding the coronavirus antigen.

55. The kit of claim 54, wherein the coronavirus antigen is a coronavirus spike protein or a peptide thereof.

56. The kit of claim 54, wherein the coronavirus antigen is a coronavirus nucleocapsid protein or a peptide thereof.

57. The kit of claim 54, wherein the coronavirus antigen comprises a coronavirus spike protein or a peptide thereof and a coronavirus nucleocapsid protein or a peptide thereof.
